(19) United States
(12) Patent Application Publication
(10) Pub. No.: US 2011/0312941 A1
(43) Pub. Date: Dec. 22, 2011

(54) 1,4-DISUBSTITUTED PIPERIDINES AS VASOPRESSIN RECEPTOR VIA ANTAGONISTS

(75) Inventors: Andrzej Roman Batt, Southampton (GB); Celine Marguerite Simone Heeney, Southampton (GB)

(73) Assignee: VANTIA LIMITED, Southampton (GB)

(21) Appl. No.: 13/201,054

(22) PCT Filed: Feb. 25, 2010
(86) PCT No.: PCT/GB2010/000323

§ 371 Date: Aug. 11, 2011

Related U.S. Application Data
(60) Provisional application No. 61/155,961, filed on Feb. 27, 2009.

(30) Foreign Application Priority Data
Feb. 27, 2009 (GB) 0903439.5

(51) Int. Cl. A61K 31/55; A61K 31/5517; A61P 15/00; A61P 15/06; A61P 9/12; A61P 9/04; A61P 25/00; A61P 3/06; A61P 35/00; A61P 25/24; A61P 25/22; A61P 1/16; C07D 487/04; A61P 7/00

(52) U.S. Cl. 514/215; 540/578; 540/557; 540/561; 514/220

(57) ABSTRACT

The present invention provides compounds of formula (1) compositions comprising such compounds; the use of such compounds in therapy (such as in the treatment of dysmenorrhoea); and methods of treating patients with such compounds; wherein A and G are as defined herein.

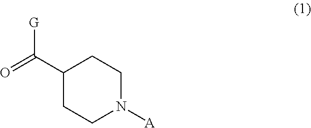

This invention relates to V₁ₐ antagonists and to processes for the preparation of, intermediates used in the preparation of, compositions containing and the uses of, such derivatives.

### BACKGROUND

The neurophyseal hormones vasopressin (VP) and oxytocin (OT) are cyclic nonapeptides secreted by the posterior pituitary gland.

Only one OT receptor has so far been well characterised, while three VP receptors are known. These are designated the V₁ₐ, V_{1b} and V₂ receptors.

Vasopressin acts on the blood vessels, where it is a potent vasoconstrictor, and on the kidneys, where it promotes water reuptake leading to an antidiuretic effect.

The V₁ₐ, V_{1b}, and V₂, as well as the OT receptors, are members of the super-family of seven transmembrane receptors known as G-protein coupled receptors. The V₁ₐ receptor mediates phospholipase C activation and intracellular calcium mobilisation. Localisation of the receptors includes blood platelets, blood vessels, hepatocytes, brain and uterus-cervix. Thus a V₁ₐ antagonist may have effects on any or all of these tissues. For example, selective V₁ₐ antagonists have been cited as having clinical utility in dysmenorrhoea, pre-term labour, hypertension, Raynaud's disease, brain oedema, motion sickness, hyperlipemia, small cell lung cancer, depression, anxiety, hyponatremia, liver cirrhosis and congestive heart failure.

With respect to dysmenorrhoea it has been proposed that myometrial activity is markedly increased in women with dysmenorrhoea during menstruation. It is proposed that the myometrial ischemia caused by increased uterine contractility might explain the menstrual pain. Furthermore, on the first day of menstruation, higher plasma concentrations of vasopressin have been measured in dysmenorroeic women than in controls.

In healthy women without dysmenorrhoea, intravenous in-fusion of lysine-vasopressin resulted in decreased uterine blood flow, increased uterine contractility and slight to moderate dysmenorrhoea-like pain, these effects being inhibited by a selective human V₁ₐ receptor antagonist (British Journal of Obstetrics And Gynaecology 1997, 104(4), 471). Also, it is known that vasopressin contracts human uterine arteries in a dose-dependent and V₁ₐ-mediated fashion.

The above evidence suggests that a V₁ₐ antagonist would be an appropriate and effective treatment for dysmenorrhoea (primary dysmenorrhoea and/or secondary dysmenorrhoea). Further evidence is taken from the clinical study carried out on the selective V₁ₐ antagonist SR49059 (Brouard, R. et al. British Journal of Obstetrics and Gynaecology 2000, 107(5), 614. It was found that there was a dose-related decrease in pain and a dose-related decrease in the amount of additional pain-killer taken compared to patients taking placebo.

WO 03/016316 A1 describes a number of compounds which are claimed to be oxytocin agonists and to find use in the treatment of male erectile dysfunction. No V₁ₐ antagonist activity is reported.

EP 1 449 844 A1 describes a number of compounds which are claimed to be V₁ₐ antagonists and to find use in the treatment of primary dysmenorrhoea.

WO 2006/021213 A2 describes compounds which are V₁ₐ antagonists and find use in the treatment of primary dysmenorrhoea.

There exists a need for treatments for conditions which are associated with the V₁ₐ receptors. Therefore, there continues to be a need for alternative V₁ₐ antagonists.

### SUMMARY OF THE INVENTION

In an aspect, the present invention provides a compound of formula (1):

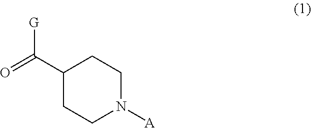

wherein,
G is a fused azepine selected from formula (2), (3), or (4),

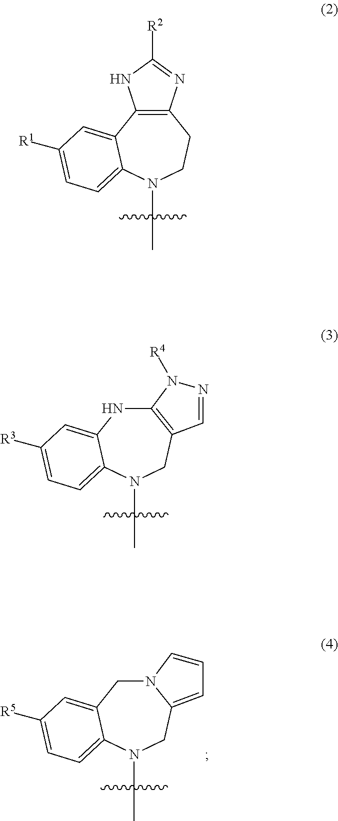

wherein,
R¹ is H, halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-;
R² is (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-;
R³ is H, halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-;
R⁴ is (C₁-C₁₀)alkyl, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-;
R⁵ is H, halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-; and
A is phenyl or a 5- or 6-membered aromatic ring containing 1, 2 or 3 N atoms, wherein said phenyl or said 5- or 6-membered ring are optionally substituted with 1 to 3 substituents independently chosen from the group consisting of halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl-, heteroaryl(C₁-C₄)alkyl-, CF₃, CN, NO₂, OH, CO₂R^{d} and NR^{d}R^{e}, wherein
each alkyl may independently be optionally substituted with 1 or 2 substituents independently selected from (C₃-C₁₀)cycloalkyl, (C₁-C₆)alkoxy, OH, CN, CF₃, CO₂R^{p}, halo and NR^{p}R^{q};
each alkenyl may independently be optionally substituted with 1 or 2 substituents independently selected from (C₃-C₁₀)cycloalkyl, (C₁-C₆)alkoxy, OH, CN, CF₃, CO₂R^{r}, halo and NR^{r}R^{s};
each alkoxy may independently be optionally be substituted with 1 or 2 substituents independently selected from (C₃-C₁₀)cycloalkyl, OH, CN, CF₃, CO₂R^{t}, halo and NR^{t}R^{u};
each cycloalkyl is independently a non-aromatic mono- or bicylic hydrocarbon ring, optionally fused to an aryl group, wherein said cycloalkyl ring may optionally contain up to 2 double bonds; and wherein, unless otherwise stated, said cycloalkyl may optionally be substituted with 1 or 2 substituents independently selected from (C₁-C₆)alkyl, (C₁-C₆)alkoxy, OH, CN, CF₃, CO₂R^{v}, halo and NR^{v}R^{w};
each heterocycloalkyl is independently a C-linked or N-linked non-aromatic, 3-10 membered mono- or bicyclic ring, wherein said heterocycloalkyl ring may contain 1, 2 or 3 ring members independently selected from N, NR^{x}, S(O)_{y} and O; and said heterocycloalkyl ring may optionally contain 1 or 2 double bonds, and is optionally substituted on carbon with 1 or 2 substituents independently selected from (C₁-C₆)alkyl, (C₁-C₆)alkoxy, OH, CN, CF₃, halo, CO₂R^{x}, NR^{x}R^{y} and aryl;
aryl is a 6 or 10 membered aromatic mono- or bicyclic ring system; wherein, unless otherwise stated, each occurrence of aryl may be optionally substituted with up to 5 substituents independently selected from (C₁-C₆)alkyl, (C₁-C₆)alkoxy, OH, halo, CN, CO₂R^{a}, CF₃ and NR^{a}R^{z};
heteroaryl is a 5, 6, 9 or 10 membered aromatic mono- or bicyclic ring system, containing 1 or 2 N atoms and, optionally, an NR^{b} ring member, an S or an O atom; or containing one NR^{b} ring member and an S or an O atom; or containing one NR^{b} ring member; or containing one S atom; or containing one O atom; wherein, unless otherwise stated, said heteroaryl may be optionally substituted with 1, 2 or 3 substituents independently selected from (C₁-C₆)alkyl, (C₁-C₆)alkoxy, OH, halo, CN, CO₂R^{b}, CF₃ and NR^{b}R^{c}; wherein
R^{p}, R^{q}, R^{r}, R^{s}, R^{t}, R^{u}, R^{x}, R^{y}, R^{a}, R^{z}, R^{b}, R^{c}, R^{d} and R^{e} are each independently selected from H and (C₁-C₆)alkyl; and
y is 0, 1 or 2;
   and pharmaceutically acceptable salts and solvates thereof.

In an aspect the present invention provides a prodrug of a compound of formula (I) as herein defined, or a pharmaceutically acceptable salt thereof.

In an aspect the present invention provides an N-oxide of a compound of formula (I) as herein defined, or a prodrug or pharmaceutically acceptable salt thereof.

It will be understood that certain compounds of the present invention may exist in solvated, for example hydrated, as well as unsolvated forms. It is to be understood that the present invention encompasses all such solvated forms.

In an aspect the present invention provides a compound of formula (1), wherein:

R¹ is H, halo or (C₁-C₆)alkyl;
R² is (C₁-C₆)alkyl, aryl, heterocycloalkyl or aryl(C₁-C₄)alkyl-;
R³ is H, halo or (C₁-C₆)alkyl;
R⁴ is (C₁-C₆)alkyl, aryl, aryl(C₁-C₄)alkyl-;
R⁵ is H, halo or (C₁-C₆)alkyl;

### A is

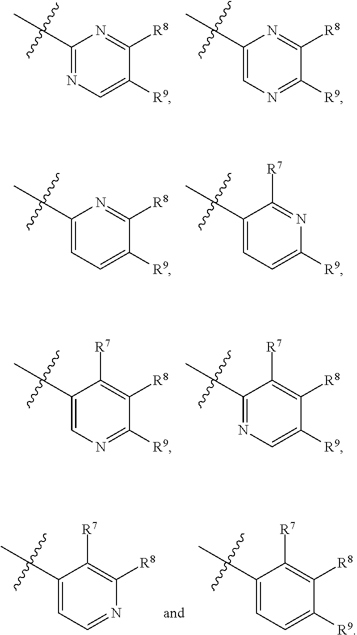 [0028] wherein
   R⁷ is H, halo, (C₁-C₆)alkyl, (C₁-C₆)alkoxy, CN, CH₂NH₂, NO₂ or NH₂;
   R⁸ is H, halo, (C₁-C₆)alkyl, (C₁-C₆)alkoxy, CN, NO₂, CF₃ or Ph; and
   R⁹ is H, halo, (C₁-C₆)alkyl, (C₁-C₆)alkoxy, CN, CH₂NH₂, NO₂, NH₂ or CF₃;
   wherein G, alkyl, alkoxy, aryl and heterocycloalkyl are as previously defined;
   and pharmaceutically acceptable salts and solvates thereof.

In an aspect the present invention provides a compound of formula (1), wherein:

### R¹ is H, F, Cl or Me;

R² is Me, Et, n-Pr, n-Bu, i-Bu, cyclopropyl, cyclohexyl, phenyl, (2-fluoro)-phenyl, (3-fluoro)-phenyl, benzyl, tetrahydropyranyl, pyrrolidinyl, piperidinyl, morpholinyl or N-methylpiperazinyl;

### R³ is H, F, Cl or Me;

R⁴ is Me, Et, t-butyl, cyclohexyl, phenyl or benzyl;

### R⁵ is H, F or Me;

### R⁶ is H, F, Cl, Me, OMe, CN, CH₂NH₂, NO₂ or NH₂;

### R⁷ is H, F, Cl, Me, OMe, CN, NO₂, CF₃ or Ph; and

R⁸ is H, F, Cl, Me, OMe, CN, CH₂NH₂, NO₂, NH₂, CF₃, Et, n-Pr or i-Pr;
wherein G is as previously defined;
and pharmaceutically acceptable salts and solvates thereof.

The present invention additionally comprises the following aspects:

i) A compound of formula (1), as previously defined, wherein G is a fused azepine of general formula 2.
ii) A compound of formula (1), as previously defined, wherein G is a fused azepine of general formula 3.
iii) A compound of formula (1), as previously defined, wherein G is a fused azepine of general formula 4.
iv) A compound of formula (1), as defined in aspect i), wherein R¹ is H, halo, (C₁-C₁₀)alkyl, (C₁-C₆)alkoxy, (C₃-C₁₀)cycloalkyl, aryl or aryl(C₁-C₄)alkyl-.
v) A compound of formula (1), as defined in aspect i), wherein R¹ is H, halo or (C₁-C₆)alkyl.
vi) A compound of formula (1), as defined in aspect i), wherein R¹ is H, F, Cl or Me.
vii) A compound of formula (1), as defined in aspect i), wherein R¹ is F or Me.
viii) A compound of formula (1), as defined in any one of aspects i) and iv)-vii), wherein R² is (C₁-C₁₀)alkyl, (C₁-C₆)alkoxy, (C₃-C₁₀)cycloalkyl, heterocycloalkyl, aryl, aryl(C₁-C₄)alkyl- or heteroaryl(C₁-C₄)alkyl-.
ix) A compound of formula (1), as defined in any one of aspects i) and iv)-vii), wherein R² is (C₁-C₆)alkyl, aryl, heterocycloalkyl or aryl(C₁-C₄)alkyl-.
x) A compound of formula (1), as defined in any one of aspects i) and iv)-vii), wherein R² is Me, Et, n-Pr, n-Bu, i-Bu, cyclopropyl, cyclohexyl, phenyl, (2-fluoro)-phenyl, (3-fluoro)-phenyl, benzyl, tetrahydropyranyl, pyrrolidinyl, piperidinyl, morpholinyl or N-methylpiperazinyl.
xi) A compound of formula (1), as defined in any one of aspects i) and iv)-vii), wherein R² is Me, Et, n-Pr, n-Bu, i-Bu, cyclopropyl, cyclohexyl, benzyl, tetrahydropyranyl, pyrrolidinyl, piperidinyl, morpholinyl or N-methylpiperazinyl.
xii) A compound of formula (1), as defined in any one of aspects i) and iv)-vii), wherein R² is morpholinyl.
xiii) A compound of formula (1), as defined in aspect ii), wherein R³ is H, halo, (C₁-C₁₀)alkyl, (C₁-C₆)alkoxy, (C₃-C₁₀)cycloalkyl, aryl or aryl(C₁-C₄)alkyl-.
xiv) A compound of formula (1), as defined in aspect ii), wherein R³ is H, halo or (C₁-C₆)alkyl.
xv) A compound of formula (1), as defined in aspect ii), wherein R³ is H, F, Cl or Me.
xvi) A compound of formula (1), as defined in any one of aspects ii) and xiii)-xv), wherein R⁴ is (C₁-C₁₀)alkyl, (C₃-C₁₀)cycloalkyl, heterocycloalkyl, aryl, aryl(C₁-C₄)alkyl- or heteroaryl(C₁-C₄)alkyl-.
xvii) A compound of formula (1), as defined in any one of aspects ii) and xiii)-xv), wherein R⁴ is (C₁-C₆)alkyl, aryl or aryl(C₁-C₄)alkyl-.
xviii) A compound of formula (1), as defined in any one of aspects ii) and xiii)-xv), wherein R⁴ is Me, Et, t-butyl, cyclohexyl, phenyl or benzyl.
xix) A compound of formula (1), as defined in aspect iii), wherein R⁵ is H, halo, (C₁-C₁₀)alkyl, (C₁-C₆)alkoxy, (C₃-C₁₀)cycloalkyl, aryl or aryl(C₁-C₄)alkyl-.
xx) A compound of formula (1), as defined in aspect iii), wherein R⁵ is H, halo or (C₁-C₆)alkyl.
xxi) A compound of formula (1), as defined in aspect iii), wherein R⁵ is H, F or Me.
xxii) A compound of formula (1), as defined in any one of the previous aspects, wherein A is a phenyl or a 6-membered aromatic ring containing 1, 2 or 3 N atoms and wherein said phenyl or said 6-membered ring are optionally substituted with 1 to 3 substituents independently chosen from the group consisting of halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl-, heteroaryl(C₁-C₄)alkyl-, CF₃, CN, NO₂, OH, CO₂R^{d} and NR^{d}R^{e}, wherein R^{d} and R^{e} are as previously defined.
xxiii) A compound of formula (1), as defined in aspect xxii), wherein A is selected from the group consisting of:

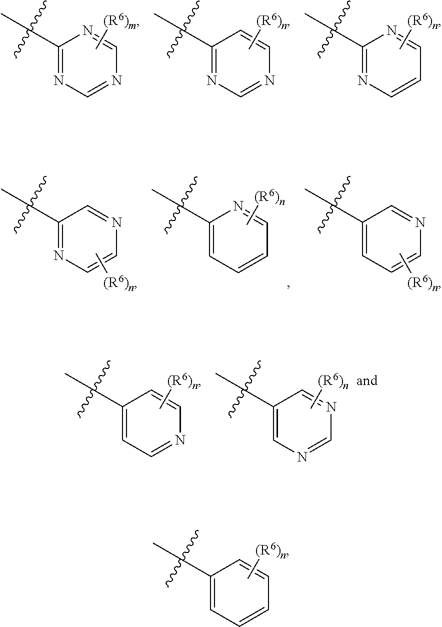

wherein m is 0, 1 or 2; and n is independently 0, 1, 2 or 3, wherein when present, each R⁶ is independently selected from halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl-, heteroaryl(C₁-C₄)alkyl-, CF₃, CN, NO₂, OH, CO₂R^{d} and NR^{d}R^{e}, wherein R^{d} and R^{e} are as previously defined.
xxiv) A compound of formula (1), as defined in aspect xxiii), wherein each R⁶ is independently selected from halo, (C₁-C₁₀)alkyl, (C₁-C₆)alkoxy, (C₃-C₁₀)cycloalkyl, aryl, aryl(C₁-C₄)alkyl-, CN, CF₃, NO₂ and NH₂.
xxv) A compound of formula (1), as defined in aspect xxiii), wherein each R⁶ is independently selected from H, F, Cl, Me, Et, n-Pr, i-Pr, OMe, CN, CH₂NH₂, NO₂ or NH₂, CF₃ or Ph.
xxvi) A compound of formula (1), as defined in aspect xxiii), wherein each R⁶ is independently selected from H, Me or OMe.
xxvii) A compound of formula (1), as defined in aspect xxii), wherein A is selected from the group consisting of:

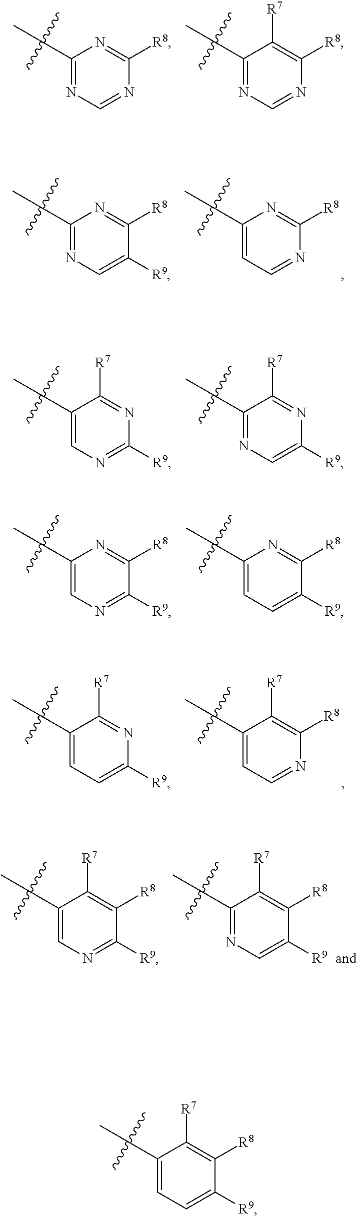

wherein each R⁷, R⁸ and R⁹ is independently selected from the group consisting of
H, halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl-, heteroaryl(C₁-C₄)alkyl-, CF₃, CN, NO₂, OH, CO₂R^{d} and NR^{d}R^{e}, wherein R^{d} and R^{e} are as previously defined.
xxviii) A compound of formula (1), as defined in aspect xxvii), wherein A is selected from the group consisting of

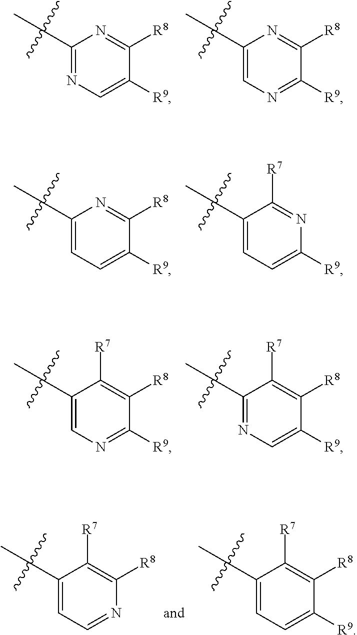

xxix) A compound of formula (1), as defined in aspect xxvii) or aspect xxviii), wherein R⁷ is H, halo, (C₁-C₆)alkyl, (C₁-C₆)alkoxy, (C₃-C₁₀)cycloalkyl, aryl, aryl(C₁-C₄)alkyl-, CF₃, CN, NO₂ or NH₂.
xxx) A compound of formula (1), as defined in aspect xxvii) or aspect xxviii), wherein R⁷ is H, F, Cl, Me, OMe, CN, CH₂NH₂, NO₂ or NH₂.
xxxi) A compound of formula (1), as defined in aspect xxvii) or aspect xxviii), wherein R⁷ is H, Me or OMe.
xxxii) A compound of formula (1), as defined in any one of aspects xxvii)-xxxi), wherein R⁸ is H, halo, (C₁-C₆)alkyl, (C₁-C₆)alkoxy, (C₃-C₁₀)cycloalkyl, aryl, aryl(C₁-C₄)alkyl-, CF₃, CN, NO₂ or NH₂.
xxxiii) A compound of formula (1), as defined in any one of aspects xxvii)-xxxi), wherein R⁸ is H, halo, (C₁-C₆)alkyl, (C₁-C₆)alkoxy, CN, NO₂, CF₃ or Ph.
xxxiv) A compound of formula (1), as defined in any one of aspects xxvii)-xxxi), wherein R⁸ is H, F, Cl, Me, OMe, CN, NO₂, CF₃ or Ph.
xxxv) A compound of formula (1), as defined in any one of aspects xxvii)-xxxi), wherein R⁸ is H, Me or OMe.
xxxvi) A compound of formula (1), as defined in any one of aspects xxvii)-xxxv), wherein R⁹ is H, halo, (C₁-C₆)alkyl, (C₁-C₆)alkoxy, (C₃-C₁₀)cycloalkyl, aryl, aryl(C₁-C₄)alkyl-, CN, NO₂ or NH₂.
xxxvii) A compound of formula (1), as defined in any one of aspects xxvii)-xxxv), wherein R⁹ is H, F, Cl, (C₁-C₆)alkyl, (C₁-C₆)alkoxy, CF₃, CN, NO₂ or NH₂.
xxxviii) A compound of formula (1), as defined in any one of aspects xxvii)-xxxv), wherein R⁹ is H, halo, Me, OMe, CN, CH₂NH₂, NO₂, NH₂, CF₃, Et, n-Pr, or i-Pr.
xxxix) A compound of formula (1), as defined in any one of aspects xxvii)-xxxv), wherein R⁹ is H, Me or OMe.
xl) A compound of formula (1), as defined in any one of aspects xxiii)-xxvi), wherein m and n are each 1.
xli) A compound of formula (1), as defined in any one of aspects xxvii)-xxxix), wherein R⁸ and R⁹ are each H when R⁷ is not H.
xlii) A compound of formula (1), as defined in any one of aspects xxvii)-xxxix), wherein R⁷ and R⁸ are each H when R⁹ is not H.
xliii) A compound of formula (1), as defined in any one of aspects xxvii)-xxxix), wherein R⁷ and R⁹ are each H when R⁸ is not H.
xliv) A compound of formula (1), as defined in aspect xxvii) or xxviii), wherein at least one of R⁷-R⁹ is H.
xlv) A compound of formula (1), as defined in aspect xxvii) or xxviii), wherein R⁷-R⁹ are each H.
xlvi) A compound of formula (1), as defined in aspect xxii), wherein A is selected from the group consisting of:

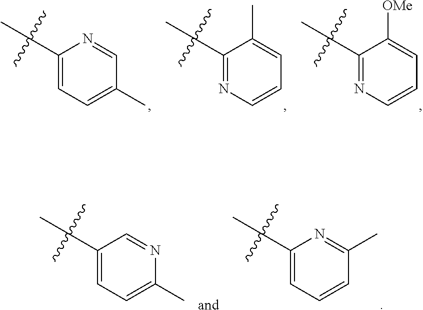

In an aspect, the present invention provides a compound of formula (1) selected from the group consisting of:
(9-Methyl-2-morpholin-4-yl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(3′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)methanone;
(9-Fluoro-2-morpholin-4-yl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(6′-methyl-3,4,5,6-tetrahydro-2H-[1,3′]bipyridinyl-4-yl)-methanone;
(2-Morpholin-4-yl-4,5-dihydro-3H-1,3,6-triaza-benzo[e]azulen-6-yl)-(3,4,5,6-tetrahydro-2H-[1,2′]bipyridinyl-4-yl)-methanone;
(3′-Methyl-3,4,5,6-tetrahydro-2H-[1,2′]bipyridinyl-4-yl)-(2-morpholin-4-yl-4,5-dihydro-1H-1,3,6-triaza-benzo[e]azulen-6-yl)-methanone;
(4′-Methyl-3,4,5,6-tetrahydro-2H-[1,2′]bipyridinyl-4-yl)-(2-morpholin-4-yl-4,5-dihydro-3H-1,3,6-triaza-benzo[e]azulen-6-yl)-methanone;
(5′-Methyl-3,4,5,6-tetrahydro-2H-[1,2′]bipyridinyl-4-yl)-(2-morpholin-4-yl-4,5-dihydro-3H-1,3,6-triaza-benzo[e]azulen-6-yl)-methanone;
(9-Fluoro-2-morpholin-4-yl-4,5-dihydro-1H-1,3,6-triaza-benzo[e]azulen-6-yl)-(3,4,5,6-tetrahydro-2H-[1,2′]bipyridinyl-4-yl)-methanone;
(9-Fluoro-2-morpholin-4-yl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(9-Fluoro-2-morpholin-4-yl-4,5-dihydro-1H-1,3,6-triaza-benzo[e]azulen-6-yl)-(4′-methyl-3,4,5,6-tetrahydro-2H-[1,2′]bipyridinyl-4-yl)-methanone;
(9-Fluoro-2-morpholin-4-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(2-Ethyl-9-methyl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(2-Ethyl-9-methyl-4,5-dihydro-3H-1,3,6-triaza-benzo[e]azulen-6-yl)-(4′-methyl-3,4,5,6-tetrahydro-2H-[1,2′]bipyridinyl-4-yl)-methanone;
(2-Ethyl-9-methyl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(9-Methyl-2-propyl-4,5-dihydro-3H-1,3,6-triaza-benzo[e]azulen-6-yl)-(3,4,5,6-tetrahydro-2H-[1,2′]bipyridinyl-4-yl)-methanone;
(9-Methyl-2-propyl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(9-Methyl-2-propyl-4,5-dihydro-1H-1,3,6-triaza-benzo[e]azulen-6-yl)-(4′-methyl-3,4,5,6-tetrahydro-2H-[1,2′]bipyridinyl-4-yl)-methanone;
(9-Methyl-2-propyl-4,5-dihydro-3H-1,3,6-triaza-benzo[e]azulen-6-yl)-(5′-methyl-3,4,5,6-tetrahydro-2H-[1,2′]bipyridinyl-4-yl)-methanone;
(2-Cyclopropyl-9-methyl-4,5-dihydro-3H-1,3,6-triaza-benzo[e]azulen-6-yl)-(3,4,5,6-tetrahydro-2H-[1,2′]bipyridinyl-4-yl)-methanone;
(2-Cyclopropyl-9-methyl-4,5-dihydro-3H-1,3,6-triaza-benzo[e]azulen-6-yl)-(5′-methyl-3,4,5,6-tetrahydro-2H-[1,2′]bipyridinyl-4-yl)-methanone;
(2-Butyl-9-methyl-4,5-dihydro-3H-1,3,6-triaza-benzo[e]azulen-6-yl)-(3,4,5,6-tetrahydro-2H-[1,2′]bipyridinyl-4-yl)-methanone;
(2-Butyl-9-methyl-4,5-dihydro-3H-1,3,6-triaza-benzo[e]azulen-6-yl)-(5′-methyl-3,4,5,6-tetrahydro-2H-[1,2′]bipyridinyl-4-yl)-methanone;
(2-Isobutyl-9-methyl-4,5-dihydro-3H-1,3,6-triaza-benzo[e]azulen-6-yl)-(3,4,5,6-tetrahydro-2H-[1,2′]bipyridinyl-4-yl)-methanone;
(2-Isobutyl-9-methyl-4,5-dihydro-3H-1,3,6-triaza-benzo[e]azulen-6-yl)-(5′-methyl-3,4,5,6-tetrahydro-2H-[1,2′]bipyridinyl-4-yl)-methanone;
[9-Methyl-2-(4-methyl-piperazin-1-yl)-4,5-dihydro-1H-1,3,6-triaza-benzo[e]azulen-6-yl]-(5′-methyl-3,4,5,6-tetrahydro-2[1,2′]bipyridinyl-4-yl)-methanone;
(9-Methyl-2-morpholin-4-yl-4,5-dihydro-1H-1,3,6-triaza-benzo[e]azulen-6-yl)-(3,4,5,6-tetrahydro-2H-[1,2′]bipyridinyl-4-yl)-methanone;
(9-Methyl-2-morpholin-4-yl-4,5-dihydro-3H-1,3,6-triaza-benzo[e]azulen-6-yl)-(4′-methyl-3,4,5,6-tetrahydro-2H-[1,2′]bipyridinyl-4-yl)-methanone;
(4′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)-methanone;
(5′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)-methanone;
(9-Methyl-2-morpholin-4-yl-4,5-dihydro-1H-1,3,6-triaza-benzo[e]azulen-6-yl)-(5′-trifluoromethyl-3,4,5,6-tetrahydro-2H-[1,2′]bipyridinyl-4-yl)-methanone;
(3′-Methyl-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(4′-Methyl-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(5′-Methyl-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(3′-Methoxy-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(4′-Methoxy-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(5′-Methoxy-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(9-Methyl-2-pyrrolidin-1-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(9-Chloro-2-methyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-methyl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(9-Chloro-2-ethyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-propyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-cyclopropyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-cyclohexyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-piperidin-1-yl-4,5-dihydro-1H-1,3,6-triaza-benzo[e]azulen-6-yl)-(3,4,5,6-tetrahydro-2H-[1,2′]bipyridinyl-4-yl)-methanone;
[9-Chloro-2-(4-methyl-piperazin-1-yl)-4,5-dihydro-1H-1,3,6-triaza-benzo[e]azulen-6-yl]-(3,4,5,6-tetrahydro-2H-[1,2′]bipyridinyl-4-yl)-methanone;
[9-Chloro-2-(4-methyl-piperazin-1-yl)-4,5-dihydro-1H-1,3,6-triaza-benzo[e]azulen-6-yl]-(5′-methyl-3,4,5,6-tetrahydro-2H-[1,2′]bipyridinyl-4-yl)-methanone;
(2-Benzyl-9-chloro-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-morpholin-4-yl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-morpholin-4-yl-4,5-dihydro-1H-1,3,6-triaza-benzo[e]azulen-6-yl)-(4′-methyl-3,4,5,6-tetrahydro-2H-[1,2′]bipyridinyl-4-yl)-methanone;
(9-Chloro-2-morpholin-4-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(6′-Methyl-3,4,5,6-tetrahydro-2H-[1,2′]bipyridinyl-4-yl)-(2-morpholin-4-yl-4,5-dihydro-3H-1,3,6-triaza-benzo[e]azulen-6-yl)-methanone;
(2-Ethyl-9-methyl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(9-Methyl-2-propyl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(6′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)-methanone;
(6′-Methyl-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(6′-Methoxy-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(9-Chloro-2-morpholin-4-yl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(9-Chloro-2-morpholin-4-yl-4,5-dihydro-1H-1,3,6-triaza-benzo[e]azulen-6-yl)-(6′-methoxy-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(4′-methyl-3,4,5,6-tetrahydro-2H-[1,3′]bipyridinyl-4-yl)-methanone;
(9-Methyl-2-morpholin-4-yl-4,5-dihydro-1H-1,3,6-triaza-benzo[e]azulen-6-yl)-(5′-methyl-3,4,5,6-tetrahydro-2H-[1,3′]bipyridinyl-4-yl)-methanone;
(9-Methyl-2-morpholin-4-yl-4,5-dihydro-3H-1,3,6-triaza-benzo[e]azulen-6-yl)-(2′-methyl-3,4,5,6-tetrahydro-2H-[1,3′]bipyridinyl-4-yl)-methanone;
(9-Chloro-2-methyl-4,5-dihydro-1H-1,3,6-triaza-benzo[e]azulen-6-yl)-(1-phenyl-piperidin-4-yl)-methanone;
(9-Chloro-2-ethyl-4,5-dihydro-1H-1,3,6-triaza-benzo[e]azulen-6-yl)-(1-phenyl-piperidin-4-yl)-methanone;
(9-Chloro-2-propyl-4,5-dihydro-1H-1,3,6-triaza-benzo[e]azulen-6-yl)-(1-phenyl-piperidin-4-yl)-methanone;
(9-Chloro-2-cyclopropyl-4,5-dihydro-1H-1,3,6-triaza-benzo[e]azulen-6-yl)-(1-phenyl-piperidin-4-yl)-methanone;
(9-Chloro-2-cyclohexyl-4,5-dihydro-1H-1,3,6-triaza-benzo[e]azulen-6-yl)-(1-phenyl-piperidin-4-yl)-methanone;
(9-Chloro-2-piperidin-1-yl-4,5-dihydro-1H-1,3,6-triaza-benzo[e]azulen-6-yl)-(1-phenyl-piperidin-4-yl)-methanone;
[9-Chloro-2-(4-methyl-piperazin-1-yl)-4,5-dihydro-1H-1,3,6-triaza-benzo[e]azulen-6-yl]-(1-phenyl-piperidin-4-yl)-methanone;
(9-Chloro-2-methyl-4,5-dihydro-1H-1,3,6-triaza-benzo[e]azulen-6-yl)-[1-(2-fluoro-phenyl)-piperidin-4-yl]-methanone;
(9-Chloro-2-methyl-4,5-dihydro-1H-1,3,6-triaza-benzo[e]azulen-6-yl)-[1-(4-fluoro-phenyl)-piperidin-4-yl]-methanone;
(9-Chloro-2-methyl-4,5-dihydro-1H-1,3,6-triaza-benzo[e]azulen-6-yl)-[1-(2-chloro-phenyl)-piperidin-4-yl]-methanone;
(9-Chloro-2-methyl-4,5-dihydro-1H-1,3,6-triaza-benzo[e]azulen-6-yl)-[1-(4-chloro-phenyl)-piperidin-4-yl]-methanone;
(9-Chloro-2-methyl-4,5-dihydro-1H-1,3,6-triaza-benzo[e]azulen-6-yl)-(1-o-tolyl-piperidin-4-yl)-methanone;
(9-Chloro-2-methyl-4,5-dihydro-1H-1,3,6-triaza-benzo[e]azulen-6-yl)-(1-m-tolyl-piperidin-4-yl)-methanone;
(9-Chloro-2-methyl-4,5-dihydro-1H-1,3,6-triaza-benzo[e]azulen-6-yl)-(1-p-tolyl-piperidin-4-yl)-methanone;
(9-Chloro-2-methyl-4,5-dihydro-1H-1,3,6-triaza-benzo[e]azulen-6-yl)-[1-(3-methoxy-phenyl)-piperidin-4-yl]-methanone;
(9-Chloro-2-methyl-4,5-dihydro-1H-1,3,6-triaza-benzo[e]azulen-6-yl)-[1-(4-methoxy-phenyl)-piperidin-4-yl]-methanone;
(9-Chloro-2-methyl-4,5-dihydro-1H-1,3,6-triaza-benzo[e]azulen-6-yl)-[1-(4-ethyl-phenyl)-piperidin-4-yl]-methanone;
(9-Chloro-2-methyl-4,5-dihydro-1H-1,3,6-triaza-benzo[e]azulen-6-yl)-[1-(4-propyl-phenyl)-piperidin-4-yl]-methanone;
(9-Chloro-2-methyl-4,5-dihydro-1H-1,3,6-triaza-benzo[e]azulen-6-yl)-[1-(4-isopropyl-phenyl)-piperidin-4-yl]-methanone;
(6-Chloro-3-phenyl-4,10-dihydro-3H-2,3,4,9-tetraaza-benzo[f]azulen-9-yl)-(1-pyrimidin-2-yl-piperidin-4-yl)-methanone;
(6-Chloro-3-phenyl-4,10-dihydro-3H-2,3,4,9-tetraaza-benzo[f]azulen-9-yl)-[1-(5-ethyl-pyrimidin-2-yl)-piperidin-4-yl]-methanone;
(3-Methyl-4,10-dihydro-3H-2,3,4,9-tetraaza-benzo[f]azulen-9-yl)-(3,4,5,6-tetrahydro-2H-[1,2′]bipyridinyl-4-yl)-methanone;
(3-Ethyl-6-fluoro-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(3-tert-Butyl-6-fluoro-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(3,4,5,6-tetrahydro-2H-[1,2′]bipyridinyl-4-yl)-methanone;
(3,6-Dimethyl-4,10-dihydro-3H-2,3,4,9-tetraza-benzo[f]azulen-9-yl)-(3′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(4′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(3′-methoxy-3,4,5,6-tetrahydro-2H-[1,2′]bipyridinyl-4-yl)-methanone;
(3-Benzyl-6-methyl-4,10-dihydro-3H-2,3,4,9-tetraaza-benzo[f]azulen-9-yl)-(4′-methyl-3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(3′-methyl-3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(3′-methoxy-3,4,5,6-tetrahydro-2H-[1,2′]bipyridinyl-4-yl)-methanone;
(6-Chloro-3-ethyl-4,10-dihydro-3H-2,3,4,9-tetraaza-benzo[f]azulen-9-yl)-(3′-methyl-3,4,5,6-tetrahyciro-2H-[1,2′]bipyridinyl-4-yl)-methanone;
(6-Chloro-3-ethyl-4,10-dihydro-3H-2,3,4,9-tetraaza-benzo[f]azulen-9-yl)-(4′-methyl-3,4,5,6-tetrahydro-2H-[1,2′]bipyridinyl-4-yl)-methanone;
(6-Chloro-3-ethyl-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(6-Chloro-3-phenyl-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(6′-methyl-3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(6′-methyl-3,4,5,6-tetrahydro-2H-[1,2′]bipyridinyl-4-yl)-methanone;
(6-Chloro-3-ethyl-4,10-dihydro-3H-2,3,4,9-tetraaza-benzo[f]azulen-9-yl)-(6′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-[1-(2-fluoro-phenyl)-piperidin-4-yl]-methanone;
(3,6-Dimethyl-4,10-dihydro-3H-2,3,4,9-tetraaza-benzo[f]azulen-9-yl)-(1-m-tolyl-piperidin-4-yl)-methanone;
(3,6-Dimethyl-4,10-dihydro-3H-2,3,4,9-tetraaza-benzo azulen-9-yl)-(1-p-tolyl-piperidin-4-yl)-methanone;
(3,6-Dimethyl-4,10-dihydro-3H-2,3,4,9-tetraaza-benzo[f]azulen-9-yl)-[1-(3-methoxy-phenyl)-piperidin-4-yl]-methanone;
(3,6-Dimethyl-4,10-dihydro-3H-2,3,4,9-tetraaza-benzo[f]azulen-9-yl)-[1-(4-methoxy-phenyl)-piperidin-4-yl]-methanone;
(3,6-Dimethyl-4,10-dihydro-3H-2,3,4,9-tetraaza-benzo[f]azulen-9-yl)-[1-(4-ethyl-phenyl)-piperidin-4-yl]-methanone;
(3,6-Dimethyl-4,10-dihydro-3H-2,3,4,9-tetraaza-benzo[f]azulen-9-yl)-[1-(4-trifluoromethyl-phenyl)-piperidin-4-yl]-methanone;
(6-Chloro-3-methyl-4,10-dihydro-3H-2,3,4,9-tetraaza-benzo[f]azulen-9-yl)-[1-(2-fluoro-phenyl)-piperidin-4-yl]-methanone;
(6-Chloro-3-methyl-4,10-dihydro-3H-2,3,4,9-tetraaza-benzo[f]azulen-9-yl)-(1-m-tolyl-piperidin-4-yl)-methanone;
(6-Chloro-3-methyl-4,10-dihydro-3H-2,3,4,9-tetraaza-benzo[f]azulen-9-yl)-(1-p-tolyl-piperidin-4-yl)-methanone;
(6-Chloro-3-methyl-4,10-dihydro-3H-2,3,4,9-tetraaza-benzo[f]azulen-9-yl)-[1-(3-methoxy-phenyl)-piperidin-4-yl]-methanone;
(6-Chloro-3-methyl-4,10-dihydro-3H-2,3,4,9-tetraaza-benzo[f]azulen-9-yl)-[1-(4-methoxy-phenyl)-piperidin-4-yl]-methanone;
(6-Chloro-3-methyl-4,10-dihydro-3H-2,3,4,9-tetraaza-benzo[f]azulen-9-yl)-[1-(4-ethyl-phenyl)-piperidin-4-yl]-methanone;
(6-Chloro-3-methyl-4,10-dihydro-3H-2,3,4,9-tetraaza-benzo[f]azulen-9-yl)-[1-(4-trifluoromethyl-phenyl)-piperidin-4-yl]-methanone;
(3,6-Dimethyl-4,10-dihydro-3H-2,3,4,9-tetraaza-benzo[f]azulen-9-yl)-(6′-methyl-3,4,5,6-tetrahydro-2H-[1,3′]bipyridinyl-4-yl)-methanone;
(6-Chloro-3-methyl-4,10-dihydro-3H-2,3,4,9-tetraaza-benzo azulen-9-yl)-(6′-methyl-3,4,5,6-tetrahydro-2H-[1,3′]bipyridinyl-4-yl)-methanone;
(5H,11H-Benzo[e]pyrrolo[1,2-a][1,4]diazepin-10-yl)-(3,4,5,6-tetrahydro-2H-[1,2′]bipyridinyl-4-yl)-methanone;
(5H,11H-Benzo[e]pyrrolo[1,2-a][1,4]diazepin-10-yl)-(5′-methyl-3,4,5,6-tetrahydro-2H-[1,2′]bipyridinyl-4-yl)-methanone;
(5H,11H-Benzo[e]pyrrolo[1,2-a][1,4]diazepin-10-yl)-(3,4,5,6-tetrahydro-2H-[1,3′]bipyridinyl-4-yl)-methanone;
(5H,11H-Benzo[e]pyrrolo[1,2-a][1,4]diazepin-10-yl)-(1-pyrazin-2-yl-piperidin-4-yl)-methanone;
(5H,11H-Benzo[e]pyrrolo[1,2-a][1,4]diazepin-10-yl)-(1-phenyl-piperidin-4-yl)-methanone;
(7-Fluoro-5H,11H-benzo[e]pyrrolo[1,2-a][1,4]diazepin-10-yl)-(3,4,5,6-tetrahydro-2,1-[1,2′]bipyridinyl-4-yl)-methanone;
(7-Fluoro-5H,11H-benzo[e]pyrrolo[1,2-a][1,4]diazepin-10-yl)-(1-phenyl-piperidin-4-yl)-methanone;
(7-Methyl-5H,11H-benzo[e]pyrrolo[1,2-a][1,4]diazepin-10-yl)-(3,4,5,6-tetrahydro-2H-[1,2′]bipyridinyl-4-yl)-methanone;
(7-Methyl-5H,11H-benzo[e]pyrrolo[1,2-a][1,4]diazepin-10-yl)-(1-phenyl-piperidin-4-yl)-methanone;
(5H,11H-Benzo[e]pyrrolo[1,2-a][1,4]diazepin-10-yl)-[1-(2-fluoro-phenyl)-piperidin-4-yl]-methanone;
(5H,11H-Benzo[e]pyrrolo[1,2-a][1,4]diazepin-10-yl)-[1-(3-fluoro-phenyl)-piperidin-4-yl]-methanone;
(5H,11H-Benzo[e]pyrrolo[1,2-a][1,4]diazepin-10-yl)-[1-(4-fluoro-phenyl)-piperidin-4-yl]-methanone;
(5H,11H-Benzo[e]pyrrolo[1,2-a][1,4]diazepin-10-yl)-[1-(2-chloro-phenyl)-piperidin-4-yl]-methanone;
(5H,11H-Benzo[e]pyrrolo[1,2-a][1,4]diazepin-10-yl)-[1-(4-chloro-phenyl)-piperidin-4-yl]-methanone;
(5H,11H-Benzo[e]pyrrolo[1,2-a][1,4]diazepin-10-yl)-(1-o-tolyl-piperidin-4-yl)-methanone;
(5H,11H-Benzo[e]pyrrolo[1,2-a][1,4]diazepin-10-yl)-(1-m-tolyl-piperidin-4-yl)-methanone;
(5H,11H-Benzo[e]pyrrolo[1,2-a][1,4]diazepin-10-yl)-(1-p-tolyl-piperidin-4-yl)-methanone;
(5H,11H-Benzo[e]pyrrolo[1,2-a][1,4]diazepin-10-yl)-[1-(2-methoxy-phenyl)-piperidin-4-yl]-methanone;
(5H,11H-Benzo[e]pyrrolo[1,2-a][1,4]diazepin-10-yl)-[1-(3-methoxy-phenyl)-piperidin-4-yl]-methanone;
(5H,11H-Benzo[e]pyrrolo[1,2-a][1,4]diazepin-10-yl)-[1-(4-methoxy-phenyl)-piperidin-4-yl]-methanone;
(5H,11H-Benzo[e]pyrrolo[1,2-a][1,4]diazepin-10-yl)-[1-(4-ethyl-phenyl)-piperidin-4-yl]-methanone;
(5H,11H-Benzo[e]pyrrolo[1,2-a][1,4]diazepin-10-yl)-[1-(4-propyl-phenyl)-piperidin-4-yl]-methanone;
(5H,11H-Benzo[e]pyrrolo[1,2-a][1,4]diazepin-10-yl)-[1-(4-isopropyl-phenyl)-piperidin-4-yl]-methanone;
(5H,11H-Benzo[e]pyrrolo[1,2-a][1,4]diazepin-10-yl)-[1-(4-trifluoromethyl-phenyl)-piperidin-4-yl]-methanone;
3-[4-(5H,11H-Benzo[e]pyrrolo[1,2-a][1,4]diazepine-10-carbonyl)-piperidin-1-yl]-benzonitrile;
(5H,11H-Benzo[e]pyrrolo[1,2-a][1,4]diazepin-10-yl)-[1-(3-nitro-phenyl)-piperidin-4-yl]-methanone;
[1-(2-Amino-phenyl)-piperidin-4-yl]-(5H,11H-benzo[e]pyrrolo[1,2-a][1,4]diazepin-10-yl)-methanone;
[1-(4-Amino-phenyl)-piperidin-4-yl]-(5H,11H-benzo[e]pyrrolo[1,2-a][1,4]diazepin-10-yl)-methanone;
[1-(2-Aminomethyl-phenyl)-piperidin-4-yl]-(5H,11H-benzo[e]pyrrolo[1,2-a][1,4]diazepin-10-yl)-methanone;
   and pharmaceutically acceptable salts or solvates thereof.

In an aspect, the present invention provides a compound of formula (1) selected from the group consisting of:
(9-Methyl-2-morpholin-4-yl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(3′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)methanone;
(9-Fluoro-2-morpholin-4-yl-4,5-dihydro-1H-1,3,6-triaza-benzo[e]azulen-6-yl)-(6′-methyl-3,4,5,6-tetrahydro-2H-[1,2′]bipyridinyl-4-yl)-methanone; and
(9-Methyl-2-morpholin-4-yl-4,5-dihydro-1H-1,3,6-triaza-benzo[e]azulen-6-yl)-(6′-methyl-3,4,5,6-tetrahydro-2H-[1,3′]bipyridinyl-4-yl)-methanone;
   and pharmaceutically acceptable salts or solvates thereof.

The skilled person will appreciate that each of the compounds identified above, or identified in the Examples provided herein below, taken alone or with any combination of the other identified compounds represents an independent aspect of the invention.

### Therapeutic Applications

As previously mentioned, the compounds of the present invention have a number of therapeutic applications, particularly in the treatment of diseases or conditions mediated by vasopressin V₁ₐ.

Accordingly, the present invention provides a compound of formula (1), or a pharmaceutically acceptable salt or solvate thereof, for use in therapy.

The present invention also provides for the use of a compound of formula (1) in the manufacture of a medicament for the treatment of a disease or condition mediated by vasopressin V₁ₐ receptors.

The present invention also provides a compound of formula (1) for use in the treatment of a disease or condition mediated by vasopressin V₁ₐ receptors.

The present invention also provides a method of treatment of a disease or condition mediated by vasopressin V₁ₐ receptors.

In an aspect, the disease or condition mediated by vasopressin V₁ₐ receptors is selected from dysmenorrhoea (primary dysmenorrhoea and/or secondary dysmenorrhoea), pre-term labour, hypertension, Raynaud's disease, brain oedema, motion sickness, hyperlipemia, small cell lung cancer, depression, anxiety, hyponatremia, liver cirrhosis and congestive heart failure.

In an aspect, the disease or condition mediated by vasopressin V₁ₐ receptors is dysmenorrhoea (primary dysmenorrhoea and/or secondary dysmenorrhoea).

### DEFINITIONS

The term “alkyl” includes saturated hydrocarbon residues including:
linear groups up to 10 atoms (C₁-C₁₀), or of up to 6 atoms (C₁-C₆), or of up to 4 atoms (C₁-C₄). Examples of such alkyl groups include, but are not limited, to C₁-methyl, C₂-ethyl, C₃-propyl and C₄-n-butyl.
branched groups of between 3 and 10 atoms (C₃-C₁₀), or of up to 7 atoms (C₃-C₇), or of up to 4 atoms (C₃-C₄). Examples of such alkyl groups include, but are not limited to, C₃-iso-propyl, C₄-sec-butyl, C₄-iso-butyl, C₄-tert-butyl and C₅-neo-pentyl.
   each optionally substituted as stated above.

The term “alkenyl” includes monounsaturated hydrocarbon residues including:
linear groups of between 2 and 6 atoms (C₂-C₆). Examples of such alkenyl groups include, but are not limited to, C₂-vinyl, C₃-1-propenyl, C₃-allyl, C₄-2-butenyl
branched groups of between 3 and 8 atoms (C₃-C₈). Examples of such alkenyl groups include, but are not limited to, C₄-2-methyl-2-propenyl and C₆-2,3-dimethyl-2-butenyl.
   each optionally substituted as stated above.

The term “alkoxy” includes O-linked hydrocarbon residues including:
linear groups of between 1 and 6 atoms (C₁-C₆), or of between 1 and 4 atoms (C₁-C₄). Examples of such alkoxy groups include, but are not limited to, C₁-methoxy, C₂-ethoxy, C₃-n-propoxy and C₄-n-butoxy.
branched groups of between 3 and 6 atoms (C₃-C₆) or of between 3 and 4 atoms (C₃-C₄). Examples of such alkoxy groups include, but are not limited to, C₃-iso-propoxy, and C₄-sec-butoxy and tert-butoxy.
   each optionally substituted as stated above.

Unless otherwise stated, halo is selected from Cl, F, Br and I.

Cycloalkyl is as defined above. Conveniently cycloalkyl groups may contain from 4 to 10 carbon atoms, or from 5 to 10 carbon atoms, or from 4 to 6 carbon atoms. Examples of suitable monocyclic cycloalkyl groups include cyclopropyl, cyclobutyl, cyclopentyl, cyclohexyl, cycloheptyl, cyclopentene, cyclopenta-1,3-diene, cyclohexene and cyclohexa-1,4-diene (optionally substituted as stated above). Examples of suitable bicyclic cycloalkyl groups include decahydronaphthalene, octahydro-1H-indene (optionally substituted as stated above). Examples of suitable cycloalkyl groups, when fused with aryl, include indanyl and 1,2,3,4-tetrahydronaphthyl (optionally substituted as stated above).

Heterocycloalkyl is as defined above. Examples of suitable heterocycloalkyl groups include oxiranyl, aziridinyl, azetidinyl, tetrahydrofuranyl, pyrrolidinyl, tetrahydropyranyl, piperidinyl, N-methylpiperidinyl, morpholinyl, N-methyl morpholinyl, thiomorpholinyl, thiomorpholinyl-1-oxide, thiomorpholinyl-1,1-dioxide, piperazinyl, N-methylpiperazinyl, azepinyl oxazepinyl, diazepinyl, and 1,2,3,4-tetrahydropyridinyl (optionally substituted as stated above).

Aryl is as defined above. Typically, aryl will be optionally substituted with 1, 2 or 3 substituents. Optional substituents are seleted from those stated above. Examples of suitable aryl groups include phenyl and naphthyl (each optionally substituted as stated above).

Heteroaryl is as defined above. Examples of suitable heteroaryl groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl and isoquinolinyl (optionally substituted as stated above).

Alternatively, heteroaryl is a 5, 6, 9 or 10 membered aromatic mono- or bicyclic ring system, containing 1 or 2 N atoms and, optionally, an NR^{b} ring member, or one NR^{b} ring member and an S or an O atom, or one S atom, or one O atom; wherein, unless otherwise stated, said heteroaryl may be optionally substituted with 1, 2 or 3 substituents independently selected from (C₁-C₆)alkyl, (C₁-C₆)alkoxy, OH, halo, CN, CO₂R^{b}, CF₃ and NR^{b}R^{c}.

The term “C-linked”, such as in “C-linked heterocycloalkyl”, means that the heterocycloalkyl group is joined to the remainder of the molecule via a ring carbon atom.

The term “N-linked”, such as in “N-linked heterocycloalkyl”, means that the heterocycloalkyl group is joined to the remainder of the molecule via a ring nitrogen atom.

The term “O-linked”, such as in “O-linked hydrocarbon residue”, means that the hydrocarbon residue is joined to the remainder of the molecule via an oxygen atom.

In groups such as aryl(C₁-C₄)alkyl-, “-” denotes the point of attachment of the group to the remainder of the molecule.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, fumarates, hippurates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming pro-drugs are described in ‘The Practice of Medicinal Chemistry, 2^{nd} Ed. pp 561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

In the context of the present invention, references herein to “treatment” include references to curative, palliative and prophylactic treatment.

### General Methods

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, evaporative drying, melt congealing and extrusion. Conventional drying processes including static/dynamic oven, infrared, microwave or radio frequency drying may be used to assist in the formation of the above crystalline and amorphous products.

They may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of formula (1), or a pharmaceutically acceptable salt or solvate thereof, and a pharmaceutically acceptable carrier, diluent or excipient.

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solid and liquid (including multiple phases or dispersed systems) such as tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Formulations suitable for oral administration may also be designed to deliver the compounds of formula (I) in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but restricted to glucose, manitol, sorbitol, etc.) salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of co-solvents and/or solubility-enhancing agents such as surfactants, micelle structures and cyclodextrins.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

Formulations for inhaled/intranasal administration may be formulated to be immediate and/or modified release using, for example, PGLA. Modified release formulations include delayed-, sustained-, pulsed-, controlled-, targeted and programmed release.

Inasmuch as it may desirable to administer a combination of active compounds, for example, for the purpose of treating a particular disease or condition, it is within the scope of the present invention that two or more pharmaceutical compositions, at least one of which contains a compound of formula (I), may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

The compounds of the invention may be administered alone or in combination with one or more other compounds of the invention or in combination with one or more other drugs (or as any combination thereof). The compounds of the present invention may be administered in combination with an oral contraceptive. Thus in a further aspect of the invention, there is provided a pharmaceutical product containing an V₁ₐ antagonist and an oral contraceptive as a combined preparation for simultaneous, separate or sequential use in the treatment of dysmenorrhoea.

The compounds of the present invention may be administered in combination with a PDE5 inhibitor. Thus in a further aspect of the invention, there is provided a pharmaceutical product containing a V₁ₐ antagonist and a PDEV inhibitor as a combined preparation for simultaneous, separate or sequential use in the treatment of dysmenorrhoea.

PDEV inhibitors useful for combining with V₁ₐ antagonists include, but are not limited to:
(i) 5-[2-ethoxy-5-(4-methyl-1-piperazinylsulphonyl)phenyl]-1-methyl-3-n-propyl-1,6-dihydro-7,4-pyrazolo[4,3-d]pyrimidin-7-one (sildenafil, e.g. as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/271 13, Example 8); 5-[2-/so-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO01/271 13, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/271 13, Example 66); 5-(5-Acetyl-2-propoxy-3-pyridinyl)-3-ethyl-2-(1-isopropyl-3-azetidinyl)-2,6-dihydro-7H-pyrazolo[4,3-c(]pyrimidin-7-one (see WO 01/271 12, Example 124); 5-(5-Acetyl-2-butoxy-3-pyridinyl)-3-ethyl-2-(1-ethyl-3-azetidinyl)-2,6-dihydro-7H-pyrazolo[4,3-c(]pyrimidin-7-one (see WO 01/27112, Example 132); (6R,12aR)-2,3,6,7,12,12a-hexahydro-2-methyl-6-(3,4-methylenedioxyphenyl)pyrazino[2′,1′: 6,1]pyrido[3,4-b]indole-1,4-dione (tadalafil, IC-351, Cialis®), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; the compound of example 11 of published international application WO93/07124 (EISAI); compounds 3 and 14 from Rotella D P, J. Med. Chem., 2000, 43, 1257; 4-(4˜ch!orobenzypamino-6,7,8-trimethoxyquinazoline; N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide[“DA-8159” (Example 68 of WO00/27848)]; and 7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide; and
(ii) 4-bromo-5-(pyridyImethylamino)-6-[3-(4-chlorophenyl)-propoxy]-3(2H)pyridazinone; 1-[4-[(1,3-benzodioxol-5-ylmethy)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chIorobenzyl)-2-propylindole-6-carboxylate; 4-bromo-5-(3-pyridyImethylamino)-6-(3-(4-chIorophenyl)propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-1 96960 (Glaxo Wellcome); E-8010 and E-4010 (Eisai); Bay-38-3045 & 38-9456 (Bayer); FR229934 and FR226807 (Fujisawa); and Sch-51866.

The contents of the published patent applications and journal articles and in particular the general formulae of the therapeutically active compounds of the claims and exemplified compounds therein are incorporated herein in their entirety by reference thereto.

Conveniently, the PDEV inhibitor may be selected from sildenafil, tadalafil, vardenafil, DA-8159 and 5-[2-ethoxy-5-(4-ethylpiperazin-1-ylsuIphonyl)pyridin-3-yl]-3-ethyl-2-[2-methoxyethyl]-2,6-dihydro-7H-pyrazolo[4,3-d]pyrimidin-7-one. Most conveniently the PDE5 inhibitor is sildenafil and pharmaceutically acceptable salts thereof. Sildenafil citrate is a preferred salt.

The compounds of the present invention may be administered in combination with an NO donor.

Thus in a further aspect of the invention, there is provided a pharmaceutical product containing a V₁ₐ antagonist and a NO donor as a combined preparation for simultaneous, separate or sequential use in the treatment of dysmenorrhoea.

The compounds of the present invention may be administered in combination with L-arginine, or as an arginate salt. Thus in a further aspect of the invention, there is provided a pharmaceutical product containing a V₁ₐ antagonist and L-arginine as a combined preparation for simultaneous, separate or sequential use in the treatment of dysmenorrhoea.

The compounds of the present invention may be administered in combination with a COX inhibitor.

Thus in a further aspect of the invention, there is provided a pharmaceutical product containing a V₁ₐ antagonist and a COX inhibitor as a combined preparation for simultaneous, separate or sequential use in the treatment of dysmenorrhoea.

COX inhibitors useful for combining with the compounds of the present invention include, but are not limited to:
(i) ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester (See Wenk, et al., Europ. J. Pharmacol. 453:319-324 (2002));
(ii) meloxicam, (CAS registry number 7 1125-38-7; described in U.S. Pat. No. 4,233,299), or a pharmaceutically acceptable salt or prodrug thereof;
(iii) celecoxib (U.S. Pat. No. 5,466,823), valdecoxib (U.S. Pat. No. 5,633,272), deracoxib (U.S. Pat. No. 5,521,207), rofecoxib (U.S. Pat. No. 5,474,995), etoricoxib (International Patent Application Publication No. WO 98/03484), JTE-522 (Japanese Patent Application Publication No. 9052882), or a pharmaceutically acceptable salt or prodrug thereof;
(iv) Parecoxib (described in U.S. Pat. No. 5,932,598), which is a therapeutically effective prodrug of the tricyclic Cox-2 selective inhibitor valdecoxib (described in U.S. Pat. No. 5,633,272), in particular sodium parecoxib;
(v) ABT-963 (described in International Patent Application Publication No. WO 00/24719); and

(vi) Nimesulide (described in U.S. Pat. No. 3,840,597), flosulide (discussed in J. Carter. Exp. Opin. Ther. Patents. 8(1). 21-29 (1997)), NS-398 (disclosed in U.S. Pat. No. 4,885,367), SD 8381 (described in U.S. Pat. No. 6,034,256), BMS-347070 (described in U.S. Pat. No. 6,180,651), S-2474 (described in European Patent Publication No. 595546) and MK-966 (described in U.S. Pat. No. 5,968,974).

The contents of any of the patent applications, and in particular the general formulae of the therapeutically active compounds of the claims and exemplified compounds therein, are incorporated herein in their entirety by reference thereto.

If a combination of active agents is administered, then they may be administered simultaneously, separately or sequentially.

For administration to human patients, the total daily dose of the compounds of the invention is typically in the range 0.01 mg and 1000 mg, or between 0.1 mg and 250 mg, or between 1 mg and 50 mg depending, of course, on the mode of administration. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

### Synthetic Methods

The compounds of the present invention can be prepared according to the procedures of the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention may be isolated in the form of their pharmaceutically acceptable salts, such as those described previously herein above.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (Boc), which is readily removed by treatment with an acid such as trifluoroacetic acid or hydrogen chloride in an organic solvent such as dichloromethane. Alternatively the amino protecting group may be a benzyloxycarbonyl (Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvents. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether, deprotection conditions comprise refluxing in 48% aqueous HBr for 1-24 hours, or by stirring with borane tribromide in dichloromethane for 1-24 hours. Alternatively where a hydroxy group is protected as a benzyl ether, deprotection conditions comprise hydrogenation with a palladium catalyst under a hydrogen atmosphere.

### Experimental

The invention is illustrated by the following non-limiting examples in which the following abbreviations and definitions are used:

| | |
|---|---|
| DCM | Dichloromethane |
| DMF | N,N-Dimethylformamide |
| EtOAc | Ethyl Acetate |
| h | Hours |
| LCMS | Liquid chromatography mass spectrometry |
| Me | Methyl |
| MeCN | Acetonitrile |
| MeOH | Methanol |
| Min | Minutes |
| MS | Mass spectrum |
| NMR | Nuclear magnetic resonance spectrum - NMR spectra were |
| | recorded at a frequency of 270 or 400 MHz unless otherwise |
| | indicated |
| Pet. Ether | Petroleum ether fraction boiling at 60-80° C. |
| RT | Room temperature |
| THF | Tetrahydrofuran |

All reactions were carried out under an atmosphere of nitrogen unless specified otherwise.

¹H NMR spectra were recorded on a Jeol EX 270 (270 MHz) or Brucker Avarice III (400 MHz) spectrometer with reference to deuterium solvent (CDCl₃ unless otherwise stated) and at RT. Molecular ions were obtained using LCMS which was carried out using a Chromolith Speedrod RP-18e column, 50×4.6 mm, with a linear gradient 10% to 90% 0.1% HCO₂H/MeCN into 0.1% HCO₂H/H₂O over 11 min, flow rate 1.5 mL/min. Data was collected using a Thermofinnigan Surveyor MSQ mass spectrometer with electospray ionisation in conjunction with a Thermofinnigan Surveyor LC system.

Chemical names were generated using the Autonom software provided as part of the ISIS draw package from MDL Information Systems.

Where products were purified by flash chromatography, ‘silica’ refers to silica gel for chromatography, 0.035 to 0.070 mm (220 to 440 mesh) (e.g. Merck silica gel 60), and an applied pressure of nitrogen up to 10 p.s.i accelerated column elution. Reverse phase preparative HPLC purifications were carried out using a Waters 2525 binary gradient pumping system at flow rates of typically 20 ml/min using a Waters 2996 photodiode array detector.

All solvents and commercial reagents were used as received.

### Synthesis of Intermediate Compounds

### Method A

### Compound 1:1-Pyrimidin-2-yl-piperidine-4-carboxylic acid ethyl ester

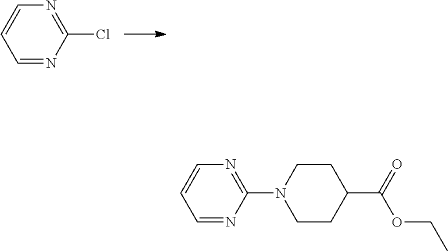

A mixture of 2-chloropyrimidine (2.28 g, 20 mmol) and ethyl isonipecotate (4.72 g, 30 mmol) in toluene (10 ml) was heated at reflux for 18 h. then cooled to RT. The mixture was diluted with EtOAc and washed with saturated aqueous NaHCO₃. The organics were separated, dried, and the solvents removed in vacuo. The residue was purified by flash chromatography on silica (eluant: EtOAc/pet. ether 40/60 v/v) to yield the title compound as a colourless oil.

Yield=4.00 g, 81%. (ESI⁺): [M+H]⁺=236.0

### Method B

### Compound 2: 1-(2-Fluorophenyl)-piperidine-4-carboxylic acid ethyl ester

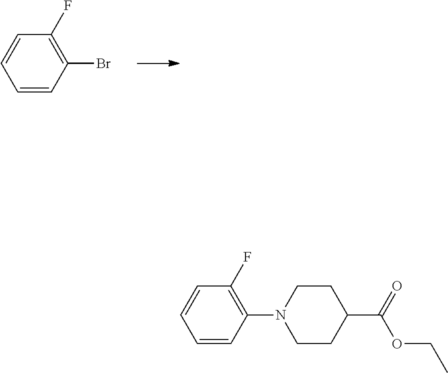

To a mixture of 1-bromo-2-fluorobenzene (5 g, 28.6 mmol) and ethyl isonipecotate (13.2 ml, 85.3 mmol [3 eq.]) in toluene (100 ml) were added sodium tert-butoxide (3.43 g, 35.7 mmol [1.25 eq.]), tris(dibenzylideneacetone)dipalladium (262 mg, 0.29 mmol [0.01 eq.]) and rac-2,2′-bis(diphenylphosphino)-1,1′-binaphthyl (534 mg, 0.86 mmol [0.03 eq.]), and the mixture was heated at 120° C. for 16 h. then cooled to RT. Water (100 ml) was added, and the layers separated. The organics were separated, dried, and the solvents removed in vacuo. The residue was purified by flash chromatography on silica (eluant: EtOAc/pet. ether 15/85 v/v) to yield the title compound as a yellow oil.

Yield=3.60 g=50.1%. (ESI⁺): [M+H]⁺=252.2

The following compounds were prepared using analogous methods to the above:

| 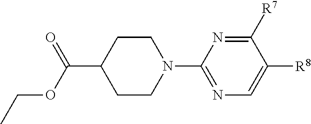 | | | | |
|---|---|---|---|---|
| | | | | (ESI⁺): |
| Compound | Method | R⁷ | R⁸ | [M + H]⁺ |
| 3 | A | CF₃ | H | 304.1 |
| 4 | A | H | 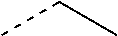 | 264.1 |

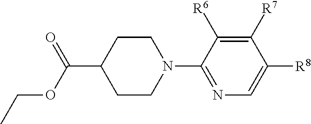

| | | | | | (ESI⁺): |
|---|---|---|---|---|---|
| Compound | Method | R⁶ | R⁷ | R⁸ | [M + H]⁺ |
| 5 | A | H | H | H | 235.1 |
| 6 | B | Me | H | H | 249.4 |
| 7 | A | H | Me | H | 249.2 |
| 8 | B | H | H | Me | 249.4 |
| 9 | B | OMe | H | H | 265.2 |
| 10 | A | H | OMe | H | 265.0 |
| 11 | B | H | H | Ome | 265.4 |
| 12 | B | H | H | CF3 | 303.4 |
| 13 | B | H | H | CN | 260.2 |

| 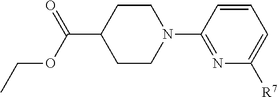 | | | |
|---|---|---|---|
| Compound | Method | R⁷ | (ESI⁺): [M + H]⁺ |
| 14 | A | Me | 249.5 |
| 15 | B | OMe | 265.4 |

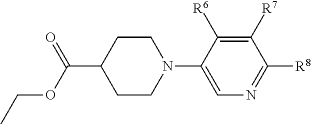

| | | | | | (ESI⁺): |
|---|---|---|---|---|---|
| Compound | Method | R⁶ | R⁷ | R⁸ | [M + H]⁺ |
| 16 | B | Me | H | H | 249.2 |
| 17 | B | H | Me | H | 249.2 |
| 18 | B | H | H | Me | 249.2 |

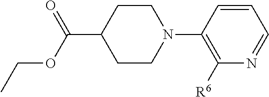

| Compound | Method | R⁶ | (ESI⁺): [M + H]⁺ |
|---|---|---|---|
| 19 | B | Me | 249.2 |

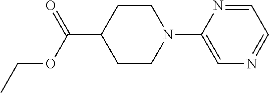

| Compound | Method | (ESI⁺): [M + H]⁺ |
|---|---|---|
| 20 | A | 236.2 |

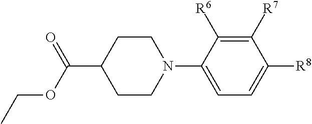

| Com- | | | | | (ESI⁺): |
|---|---|---|---|---|---|
| pound | Method | R⁶ | R⁷ | R⁸ | [M + H]⁺ |
| 21 | B | H | H | H | 234.2 |
| 22 | B | F | H | H | 252.2 |
| 23 | B | H | F | H | 252.2 |
| 24 | B | H | H | F | 252.2 |
| 25 | B | Cl | H | H | 268.2 |
| 26 | B | H | H | Cl | 268.2 |
| 27 | B | Me | H | H | 248.2 |
| 28 | B | H | Me | H | 248.1 |
| 29 | B | H | H | Me | 248.1 |
| 30 | B | OMe | H | H | 264.2 |
| 31 | B | H | OMe | H | 264.2 |
| 32 | B | H | H | OMe | 264.2 |
| 33 | B | H | H | 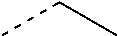 | 262.1 |
| 34 | B | H | H | 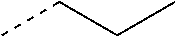 | 276.1 |
| 35 | B | H | H | 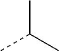 | 276.1 |
| 36 | B | H | 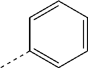 | H | 310.0 |
| 37 | B | H | H | CF₃ | 302.2 |
| 38 | A | CN | H | H | 259.2 |
| 39 | B | H | CN | H | 259.2 |
| 40 | A | H | H | CN | 259.2 |
| 41 | A | NO₂ | H | H | 279.2 |
| 42 | B | H | NO₂ | H | 279.2 |
| 43 | A | H | H | NO₂ | 279.2 |

### Compound 44: 1-Pyrimidin-2-yl-piperidine-4-carboxylic acid

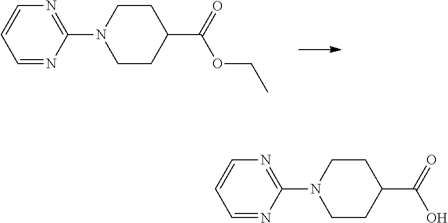

A mixture of 1-pyrimidin-2-yl-piperidine-4-carboxylic acid ethyl ester (4.00 g, 17.00 mmol) and lithium hydroxide monohydrate (1.73 g, 34.00 mmol) in THF (50 ml)/water (30 ml) at RT was stirred for 18 h, then the solvents were removed in vacuo. The residue was purified by flash chromatography on silica (eluant: chloroform/MeOH/acetic acid 50/2/1 v/v/v) to yield the title compound as a white solid.

Yield=3.00 g, 85.2%

(ESI⁺): [M+H]⁺=208.2

The following compounds were prepared using analogous methods to the above:

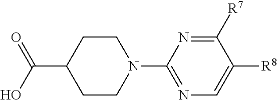

| Compound | R⁷ | R⁸ | (ESI⁺): [M + H]⁺ |
|---|---|---|---|
| 45 | CF₃ | H | 276.1 |
| 46 | H | 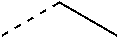 | 236.1 |

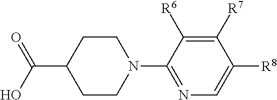

| | | | | (ESI⁺): |
|---|---|---|---|---|
| Compound | R⁶ | R⁷ | R⁸ | [M + H]⁺ |
| 47 | H | H | H | 207.2 |
| 48 | Me | H | H | 221.4 |
| 49 | H | Me | H | 221.1 |
| 50 | H | H | Me | 221.3 |
| 51 | OMe | H | H | 237.2 |
| 52 | H | OMe | H | 237.1 |
| 53 | H | H | OMe | 237.4 |
| 54 | H | H | CF3 | 275.3 |
| 55 | H | H | CN | 232.1 |

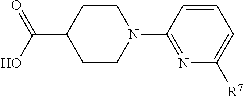

| Compound | R⁷ | (ESI⁺): [M + H]⁺ |
|---|---|---|
| 56 | Me | 221.1 |
| 57 | OMe | 237.4 |

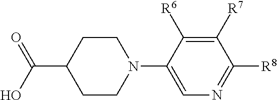

| | | | | (ESI⁺) |
|---|---|---|---|---|
| Compound | R⁶ | R⁷ | R⁸ | [M + H]⁺ |
| 58 | H | H | H | 207.1 |
| 59 | Me | H | H | 221.2 |
| 60 | H | Me | H | 221.4 |
| 61 | H | H | Me | 221.2 |

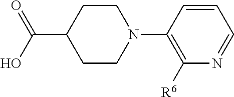

| Compound | R⁶ | (ESI⁺): [M + H]⁺ |
|---|---|---|
| 62 | Me | 221.2 |

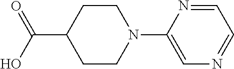

| Compound | (ESI⁺): [M + H]⁺ |
|---|---|
| 63 | 208.2 |

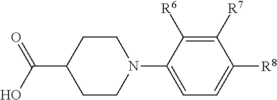

| Compound | R⁶ | R⁷ | R⁸ | [M + H]⁺ |
|---|---|---|---|---|
| 64 | H | H | H | 206.1 |
| 65 | F | H | H | 224.2 |
| 66 | H | F | H | 224.2 |
| 67 | H | H | F | 224.2 |
| 68 | Cl | H | H | 240.1 |
| 69 | H | H | Cl | 240.1 |
| 70 | Me | H | H | 220.2 |
| 71 | H | Me | H | 220.0 |
| 72 | H | H | Me | 220.0 |
| 73 | OMe | H | H | 236.0 |
| 74 | H | OMe | H | 236.2 |
| 75 | H | H | OMe | 236.2 |
| 76 | H | H | 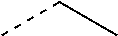 | 234.1 |
| 77 | H | H | 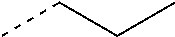 | 248.1 |
| 78 | H | H | 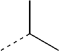 | 248.1 |
| 79 | H | 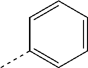 | H | 282.2 |
| 80 | H | H | CF₃ | 274.2 |
| 81 | CN | H | H | 231.0 |
| 82 | H | CN | H | 231.2 |
| 83 | H | H | CN | 231.2 |
| 84 | NO₂ | H | H | 251.1 |
| 85 | H | NO₂ | H | 251.2 |
| 86 | H | H | NO₂ | 251.1 |

The following were prepared using methods analogous to those described in Example E5 (p. 32) of WO2006021213.

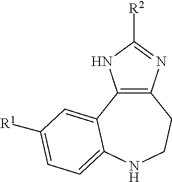

| | | | (ESI⁺): |
|---|---|---|---|
| Compound | R¹ | R² | [M + H]⁺ |
| 87 | H | Me | |
| 88 | H | 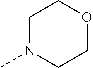 | 271.4 |
| 89 | F | 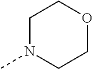 | 289.4 |
| 90 | Me | 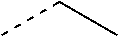 | 228.1 |
| 91 | Me | 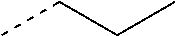 | 242.4 |
| 92 | Me | 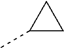 | 240.4 |
| 93 | Me | 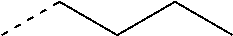 | 256.4 |
| 94 | Me | 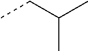 | 256.4 |
| 95 | Me | 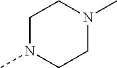 | 298.4 |
| 96 | Me | 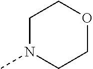 | 285.5 |
| 97 | Me | 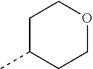 | 284.4 |
| 98 | Me | 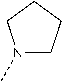 | 269.2 |
| 99 | Cl | Me | 234.1 |
| 100 | Cl | 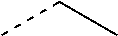 | 248.4 |
| 101 | Cl | 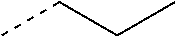 | 262.2, 264.2 |
| 102 | Cl | 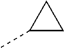 | 260.1 |
| 103 | Cl | 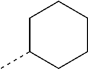 | 302.0, 304.0 |
| 104 | Cl | 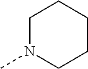 | 303.2 |
| 105 | Cl | 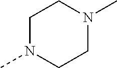 | 318.4 |
| 106 | Cl | 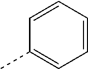 | 296.1, 298.1 |
| 107 | Cl | 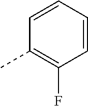 | 314.1 |
| 108 | Cl | 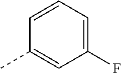 | 314.1 |
| 109 | Cl | 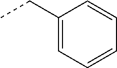 | 309.9, 312.0 |
| 110 | Cl | 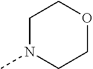 | 305.4 |

The following were prepared using methods analogous to those described in Example E1 (p. 23) of WO2006021213.

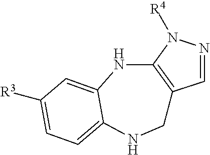

| | | | (ESI⁺): |
|---|---|---|---|
| Compound | R³ | R⁴ | [M + H]⁺ |
| 111 | H | Me | 201.3 |
| 112 | H | 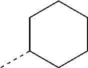 | 269.2 |
| 113 | F | Me | 219.0 |
| 114 | F | 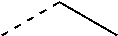 | 233.1 |
| 115 | F | 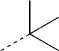 | 261.2 |
| 116 | Me | Me | 216.1 |
| 117 | Me | 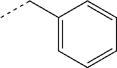 | 291.2 |
| 118 | Cl | Me | 235.2, 237.2 |
| 119 | Cl | 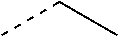 | 247.2, 249.2 |
| 120 | Cl | 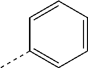 | 297.1, 299.1 |

The following compounds were prepared using methods analogous to published procedures in J. Med. Chem. 1980, p. 462.

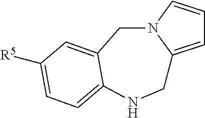

| | | (ESI⁺): |
|---|---|---|
| Compound | R⁵ | [M + H]⁺ |
| 121 | H | 185.0 |
| 122 | F | 203.2 |
| 123 | Me | 199.3 |

### SYNTHESIS OF EXAMPLES

The following examples were prepared to illustrate the invention:

### Example 1

### (9-Methyl-2-morpholin-4-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone

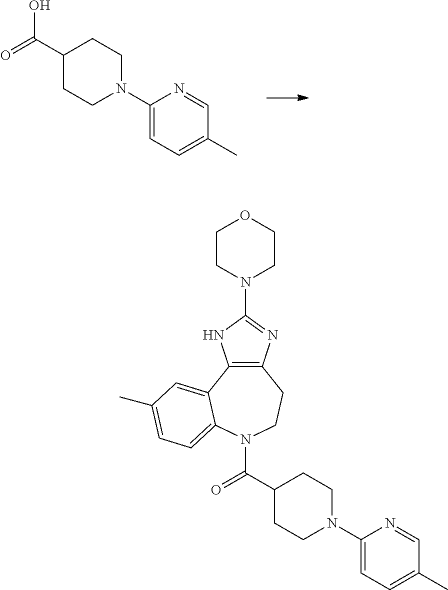

To a suspension of 5′-methyl-3,4,5,6-tetrahydro-2H-[1,2′]bipyridinyl-4-carboxylic acid (200 mg, 0.9 mmol) in DCM (5 ml) were added oxalyl chloride (0.10 ml, 1.18 mmol) and DMF (3 drops) and the mixture was stirred for 2 h. The reaction mixture was concentrated in vacuo, azeotroped with toluene (×2) and DCM (×2). The residue was redissolved in DCM (5 ml) and added to a solution of 9-methyl-2-morpholin-4-yl-1,4,5,6-tetrahydro-1,3,6-triaza-benzo[e]azulene (129 mg, 0.45 mmol) in pyridine (5 ml) at 80° C. The reaction was heated at 80° C. for 18 h and then allowed to cool to RT. The mixture was diluted with DCM (50 ml) and washed with sat. aq. NaHCO₃ (20 ml). The organics were dried, and the solvents removed in vacuo. The residue was purified by flash chromatography on silica (eluant: chloroform/MeOH 100/3 v/v) and the residue to yield a brown oil, which solidified when treated with MeCN (2 ml). The MeCN was removed in vacuo, and the resulting brown solid was triturated with diethyl ether and filtered to yield the title compound as a light brown solid (87 mg, 39.4%).

1H NMR (DMSO-d₆): 0.92 (1H, d, J=11.3 Hz), 1.09-1.18 (1H, m), 1.61-1.70 (1H, m), 1.71-1.75 (1H, m), 2.09 (3H, s), 2.25-2.33 (1H, m), 2.34 (3H, s), 2.52-2.63 (1H, m), 2.64-2.70 (3H, m), 2.96-3.09 (1H, m), 3.21-3.24 (4H, m), 3.69 (4H, t, J=4.9 Hz), 3.93 (1H, d, J=13.1 Hz), 4.20 (1H, d, J=13.1 Hz), 4.52 (1H, d, J=10.3 Hz), 6.64 (1H, d, J=8.7 Hz), 6.98 (1H, dd, J=0.9, 7.8 Hz), 7.17 (1H, d, J=7.0 Hz), 7.28 (1H, dd, J=2.3, 8.7 Hz), 7.86 (2H, d, J=2.3 Hz), 11.10 (1H, s)

(ESI⁺): [M+H]⁺=487.61

### Example 2

### (9-Methyl-2-morpholin-4-yl-4,5-dihydro-1H-1,3,6-triaza-benzo[e]azulen-6-yl)-(3′-methyl-3,4,5,6-tetrahydro-2H-[1,2′]bipyridinyl-4-yl)-methanone

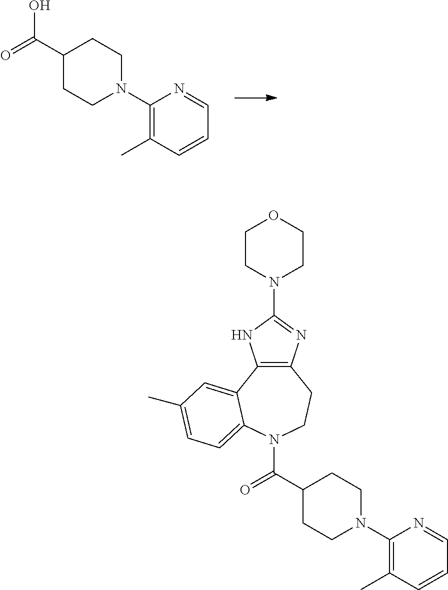

To a suspension of 3′-methyl-3,4,5,6-tetrahydro-2H-[1,2′]bipyridinyl-4-carboxylic acid (116 mg, 0.53 mmol) in DCM (5 ml) were added oxalyl chloride (0.06 ml, 0.69 mmol) and DMF (2 drops) and the mixture was stirred for 1 h. The reaction mixture was concentrated in vacuo, azeotroped with toluene (×2) and DCM (×2). The residue was redissolved in DCM (3 ml) and added to a solution of (9-methyl-2-morpholin-4-yl-1,4,5,6-tetrahydro-1,3,6-triaza-benzo[e]azulene (150 mg, 0.53 mmol) in DCM (2 ml) and triethylamine (0.148 ml, 1.05 mmol) at 0° C. The reaction was stirred at RT for 3 days. Solvents were removed in vacuo. The residue was purified by flash chromatography on silica (eluant: DCM/MeOH 98/2 to 95/5 v/v). The product was recrystallised from EtOAc/petroleum ether to give the title compound as an orange solid (85 mg, 33%).

¹H NMR (DMSO-d₆) δ 0.97-1.02 (1H, m), 1.30-1.39 (1H, m), 1.79-1.87 (2H, m), 2.14 (3H, s), 2.12-2.22 (1H, m), 2.34 (3H, s), 2.47-2.73 (4H, m), 3.04-3.14 (2H, m), 3.23-3.38 (5H, m), 3.69 (4H, t, J=4.5 Hz), 4.54-4.60 (1H, m), 6.84 (1H, dd, J=4.8, 7.2 Hz), 6.98 (1H, d, J=7.8 Hz), 7.18 (1H, d, J=7.8 Hz), 7.42 (1H, d, J=7.0 Hz), 7.86 (1H, s), 8.00 (1H, dd, J=1.5, 4.8 Hz), 11.11 (1H, s).

(ESI⁺): [M+H]⁺=487.2

### Example 3

### (3′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)-methanone

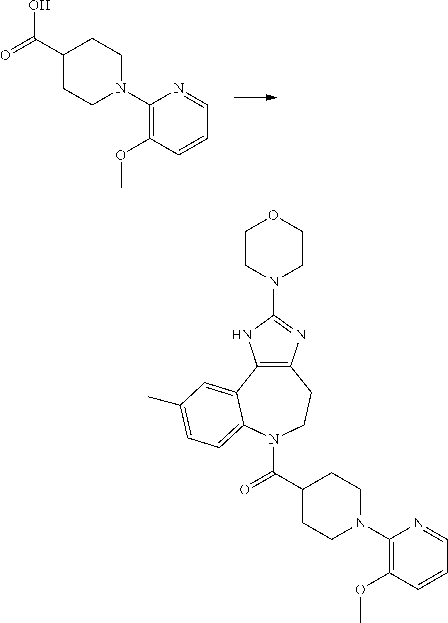

To a suspension of 3′-methoxy-3,4,5,6-tetrahydro-2H-[1,2′]bipyridinyl-4-carboxylic acid (415 mg, 1.75 mmol) in DCM (5 ml) were added oxalyl chloride (0.20 ml, 2.29 mmol) and DMF (3 drops) and the mixture was stirred for 2 h. The reaction mixture was concentrated in vacuo, azeotroped with toluene (×2) and DCM (×2). The residue was redissolved in DCM (5 ml) and added to a solution of 9-methyl-2-morpholin-4-yl-1,4,5,6-tetrahydro-1,3,6-triaza-benzo[e]azulene (129 mg, 0.45 mmol) in DCM (3 ml) and triethylamine (0.43 ml, 3.08 mmol). The reaction was stirred at RT for 72 h and the solvents removed in vacuo. The residue was purified by flash chromatography on silica (eluant: DCM/MeOH 100/2 to 100/5 v/v) and the residue was purified by prep. HPLC.

The relevant fractions were collected and the solvents removed in vacuo. The residue was partitioned between DCM and sat. aq. NaHCO₃. The organics were separated, dried and the solvents were removed in vacuo. The residue was lypophilised from MeCN/water to yield the title compound as a yellow solid (87 mg, 20%).

¹H NMR (DMSO-d₆): δ 0.94 (1H, d, J=12.1 Hz), 1.25-1.35 (1H, m), 1.73-1.86 (2H, m), 2.19 (1H, t, J=12.7 Hz), 2.34 (3H, s), 2.46-2.53 (1H, m), 2.57-2.71 (3H, m), 3.01-3.09 (1H, m), 3.25 (4H, brs), 3.63 (1H, d, J=12.5 Hz), 3.69 (4H, t, J=4.7 Hz), 3.73 (3H, s), 3.89 (1H, d, J=12.5 Hz), 4.55 (1H, d, J=8.0 Hz), 6.80 (1H, dd, J=4.7, 8.0 Hz), 6.97 (1H, dd, J=1.2, 8.0 Hz), 7.15 (1H, dd, J=1.2, 8.0 Hz), 7.16-7.18 (1H, m), 7.69 (1H, dd, J=1.2, 4.7 Hz), 7.85 (1H, brs), 11.13 (1H, brs).

(ESI⁺): [M+H]⁺=503.2

### Example 4

### (9-Methyl-2-morpholin-4-yl-4,5-dihydro-1H-1,3,6-triaza-benzo[e]azulen-6-yl)-(6′-methyl-3,4,5,6-tetrahydro-2H-[1,3′]bipyridinyl-4-yl)-methanone

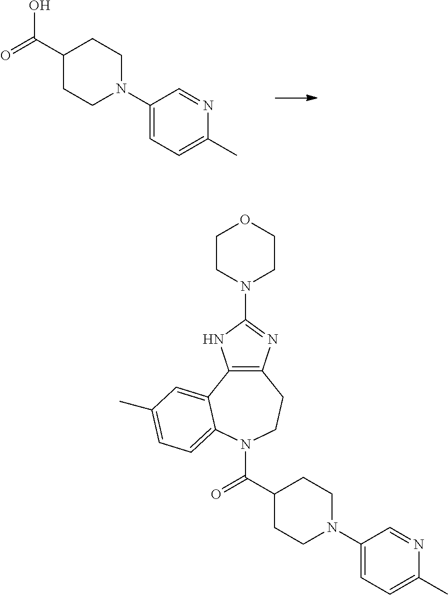

To a suspension of 6′-methyl-3,4,5,6-tetrahydro-2H-[1,3′]bipyridinyl-4-carboxylic acid (mg, 1.76 mmol) in DCM (5 ml) were added oxalyl chloride (0.20 ml, 2.29 mmol) and DMF (2 drops) and the mixture was stirred for 2 h. The reaction mixture was concentrated in vacuo, azeotroped with toluene (×2) and DCM (×2). The residue was redissolved in DCM (5 ml) and added to a solution of 9-methyl-2-morpholin-4-yl-1,4,5,6-tetrahydro-1,3,6-triaza-benzo[e]azulene (250 mg, 0.88 mmol) in DCM (3 ml) and triethylamine (0.43 ml, 3.08 mmol) at 0° C. The reaction was then stirred at RT for 24 h and the solvents removed in vacuo. The residue was purified by flash chromatography on silica (eluant: DCM/MeOH 100/2 to 100/5 v/v) and the residue was purified by prep. HPLC. The relevant fractions were collected and the solvents removed in vacuo. The residue was partitioned between chloroform and sat. aq. NaHCO₃. The organics were separated, dried and the solvents were removed in vacuo. The residue was lypophilised from MeCN/water to yield the title compound as an off-white solid (77 mg, 18%).

¹H NMR (DMSO-d₆) δ1.03-1.07 (1H, m), 1.28-1.38 (1H, m), 1.84-1.89 (2H, m), 2.21-2.28 (1H, m), 2.37 (3H, s), 2.41 (3H, s), 2.62-2.78 (4H, m), 3.09-3.18 (1H, m), 3.34 (4H, s), 3.48 (1H, d, J=12.5 Hz), 3.73 (1H, d, J=12.7 Hz), 3.76-3.78 (4H, m), 4.59-4.64 (1H, m), 7.04-7.08 (2H, m), 7.21 (1H, dd, J=2.9, 8.5 Hz), 7.27 (1H, d, J=7.9 Hz), 7.88 (1H, s), 8.10 (1H, d, J=2.9 Hz), 11.22 (1H, s).

(ESI⁺): [M+H]⁺=487.26

### Example 5

### (9-Fluoro-2-morpholin-4-yl-4,5-dihydro-1H-1,3,6-triaza-benzo[e]azulen-6-yl)-(6′-methyl-3,4,5,6-tetrahydro-2H-[1,2′]bipyridinyl-4-yl)-methanone

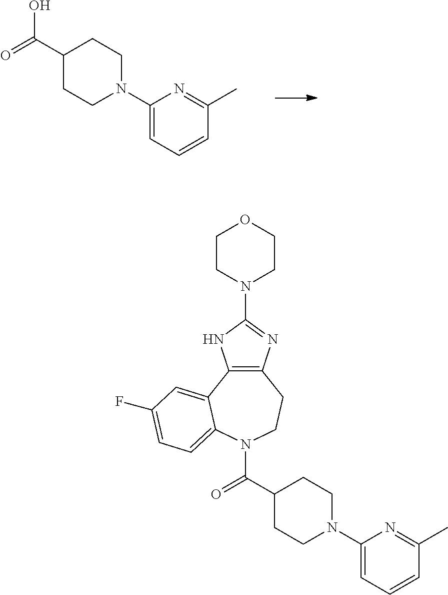

To a suspension of 6′-methyl-3,4,5,6-tetrahydro-2H-[1,2′]bipyridinyl-4-carboxylic acid (306 mg, 1.39 mmol) in DCM (5 ml) were added oxalyl chloride (0.16 ml, 1.80 mmol) and DMF (2 drops) and the mixture was stirred for 1 h. The reaction mixture was concentrated in vacuo, azeotroped with toluene (×2) and DCM (×2). The residue was redissolved in DCM (3 ml) and added to a solution of 9-fluoro-2-morpholin-4-yl-1,4,5,6-tetrahydro-1,3,6-triaza-benzo[e]azulene (200 mg, 0.69 mmol) in DCM (2 ml) and triethylamine (0.341 ml, 2.43 mmol) at 0° C. The reaction was stirred at RT for 3 h. Saturated NaHCO₃ solution was added and the layers were partitioned. The organic layer was washed with brine, dried, and the solvents were removed in vacuo. The residue was purified by flash chromatography on silica (eluant: DCM/MeOH 100/2 to 100/5 v/v). The residue was further purified further by flash chromatography on silica (eluant: DCM/MeOH 96/4tv/v) then lypophilised from MeCN/water to give the title compound as an off-white powder (64 mg, 19%).

¹H NMR (DMSO-d₆): δ 0.92 (1H, d, J=11.7 Hz), 1.05-1.15 (1H, m), 1.59-1.70 (1H, m), 1.77 (1H, d, J=11.7 Hz), 2.24 (3H, s), 2.33 (1H, dt, J=2.6, 12.0 Hz), 2.57-2.74 (4H, m), 3.01-3.11 (1H, m), 3.25 (2H, t, J=4.6 Hz), 3.30-3.35 (2H, m), 3.70 (4H, t, J=4.6 Hz), 4.01 (1H, d, J=13.1 Hz), 4.28 (1H, d, J=13.1 Hz), 4.55 (1H, dd, J=5.2, 12.5 Hz), 6.41 (1H, d, J=7.3 Hz), 6.50 (1H, d, J=8.3 Hz), 6.98 (1H, dt, J=2.6, 7.8 Hz), 7.30-7.41 (2H, m), 7.72 (1H, dd, J=3.6, 10.4 Hz), 11.23 (1H, s).

(ESI⁺): [M+H]⁺=491.5

The following examples were prepared using methods analogous to the above:

TABLE 1

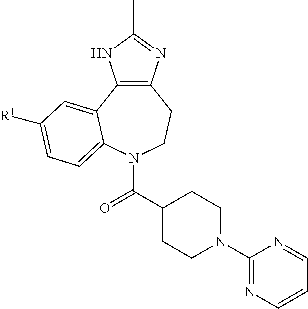

| | | (ESI⁺): |
|---|---|---|
| Example | R¹ | [M + H]⁺ |
| 6 | H | 389.2 |
| 7 | Cl | 423.3 |

TABLE 2

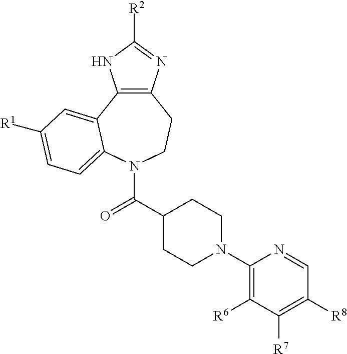

| Ex- | | | | | | (ESI⁺): |
|---|---|---|---|---|---|---|
| ample | R¹ | R² | R⁶ | R⁷ | R⁸ | [M + H]⁺ |
| 8 | H | Me | H | H | H | 388.3 |
| 9 | H | 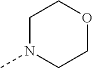 | H | H | H | 459.2 |
| 10 | H | 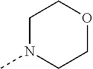 | Me | H | H | 473.5 |
| 11 | H | 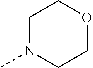 | H | Me | H | 473.2 |
| 12 | H | 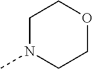 | H | H | Me | 473.3 |
| 13 | F | 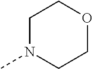 | H | H | H | 477.6 |
| 14 | F | 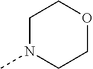 | Me | H | H | 491.5 |
| 15 | F | 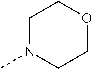 | H | Me | H | 491.5 |
| 16 | F | 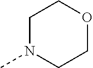 | H | H | Me | 491.6 |
| 17 | Me | 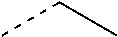 | Me | H | H | 430.2 |
| 18 | Me | 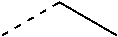 | H | Me | H | 430.2 |
| 19 | Me | 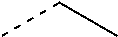 | H | Me | H | 430.2 |
| 20 | Me | 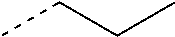 | H | H | H | 430.2 |
| 21 | Me | 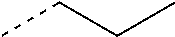 | Me | H | H | 444.3 |
| 22 | Me | 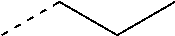 | H | Me | H | 444.3 |
| 23 | Me | 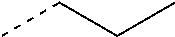 | H | H | Me | 444.5 |
| 24 | Me | 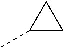 | H | H | H | 428.2 |
| 25 | Me | 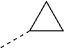 | H | H | Me | 442.2 |
| 26 | Me | 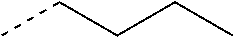 | H | H | H | 444.2 |
| 27 | Me | 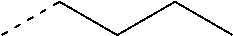 | H | H | Me | 458.3 |
| 28 | Me | 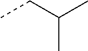 | H | H | H | 444.2 |
| 29 | Me | 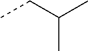 | H | H | Me | 458.3 |
| 30 | Me | 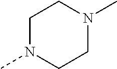 | H | H | Me | 500.3 |
| 31 | Me | 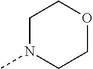 | H | H | H | 473.6 |
| 32 | Me | 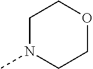 | H | Me | H | 487.2 |
| 33 | Me | 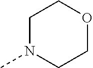 | H | OMe | H | 503.2 |
| 34 | Me | 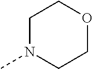 | H | H | OMe | 503.2 |
| 35 | Me | 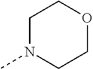 | H | H | CF₃ | 541.5 |
| 36 | Me | 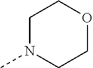 | H | H | CN | 498.2 |
| 37 | Me | 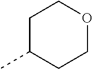 | Me | H | H | 486.3 |
| 38 | Me | 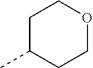 | H | Me | H | 486.3 |
| 39 | Me | 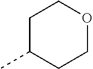 | H | H | Me | 486.3 |
| 40 | Me | 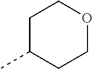 | OMe | H | H | 502.2 |
| 41 | Me | 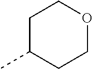 | H | OMe | H | 502.2 |
| 42 | Me | 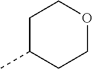 | H | H | OMe | 502.2 |
| 43 | Me | 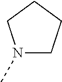 | H | H | Me | 471.3 |
| 44 | Cl | Me | H | H | H | 422.1 |
| 45 | Cl | Me | H | H | Me | 458.7 |
| 46 | Cl | 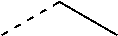 | H | H | H | 436.1 |
| 47 | Cl | 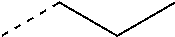 | H | H | H | 450.0, 452.1 |
| 48 | Cl | 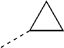 | H | H | H | 448.0 |
| 49 | Cl | 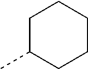 | H | H | H | 490.1, 492.1 |
| 50 | Cl | 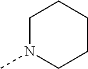 | H | H | H | 491.1, 493.1 |
| 51 | Cl | 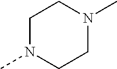 | H | H | H | 506.2, 508.2 |
| 52 | Cl | 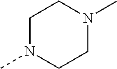 | H | H | Me | 520.5, 522.5 |
| 53 | Cl | 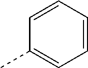 | H | H | H | 484.1, 486.1 |
| 54 | Cl | 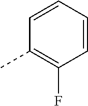 | H | H | H | 502.2 |
| 55 | Cl | 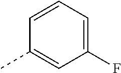 | H | H | H | 502.2 |
| 56 | Cl | 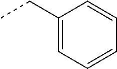 | H | H | H | 498.1, 500.1 |
| 57 | Cl | 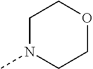 | H | H | H | 493.1, 495.1 |
| 58 | Cl | 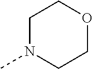 | H | Me | H | 507.2, 509.2 |
| 59 | Cl | 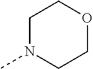 | H | H | Me | 507.2, 509.2 |

TABLE 3

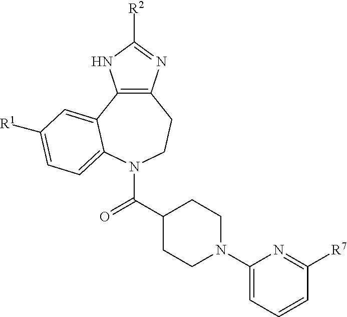

| | | | | (ESI⁺) |
|---|---|---|---|---|
| Example | R¹ | R² | R⁷ | [M + H]⁺ |
| 60 | H | 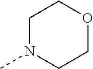 | Me | 473.21 |
| 61 | Me | 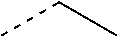 | Me | 430.23 |
| 62 | Me | 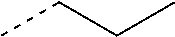 | Me | 444.24 |
| 63 | Me | 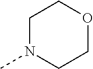 | Me | 487.23 |
| 64 | Me | 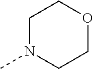 | OMe | 503.21 |
| 65 | Me | 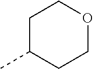 | Me | 486.25 |
| 66 | Me | 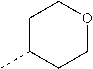 | OMe | 502.3 |
| 67 | Cl | 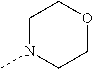 | Me | 507.2, 509.2 |
| 68 | Cl | 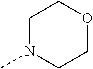 | OMe | 523.19 |

TABLE 4

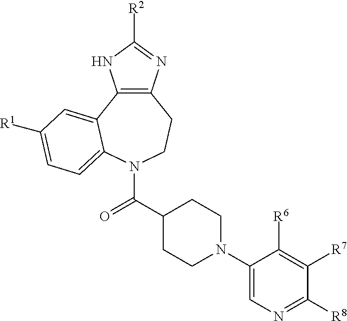

| | | | | | | (ESI⁺) |
|---|---|---|---|---|---|---|
| Example | R¹ | R² | R⁶ | R⁷ | R⁸ | [M + H]⁺ |
| 69 | Me | 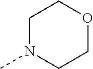 | Me | H | H | 487.26 |
| 70 | Me | 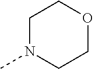 | H | Me | H | 487.5 |

TABLE 5

| 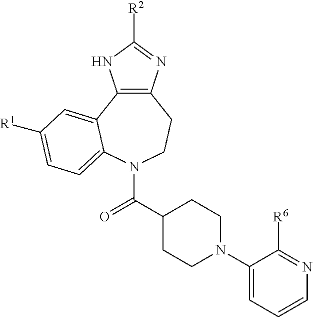 | | | | |
|---|---|---|---|---|
| | | | | (ESI⁺) |
| Example | R¹ | R² | R⁶ | [M + H]⁺ |
| 71 | Me | 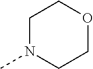 | Me | 487.28 |

TABLE 6

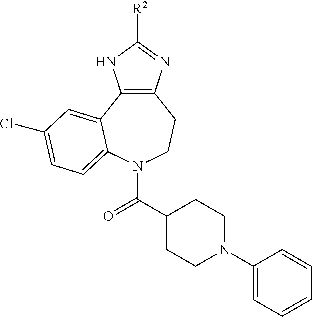

| | | (ESI⁺) |
|---|---|---|
| Example | R² | [M + H]⁺ |
| 72 | Me | 420.9 |
| 73 | 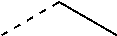 | 435.2/437.2 |
| 74 | 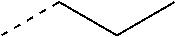 | 449.0/451.1 |
| 75 | 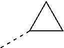 | 447.0/449.1 |
| 76 | 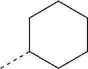 | 489.3/491.3 |
| 77 | 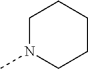 | 490.1/492.1 |
| 78 | 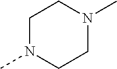 | 505.1/507.1 |
| 79 | 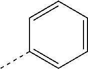 | 483.1/485.1 |
| 80 | 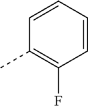 | 501.22 |
| 81 | 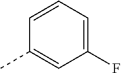 | 501.21 |
| 82 | 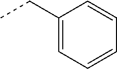 | 497.1/499.1 |

TABLE 7

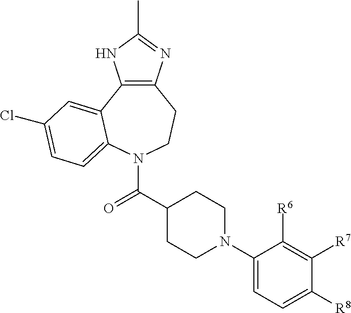

| | | | | (ESI⁺) |
|---|---|---|---|---|
| Example | R⁶ | R⁷ | R⁸ | [M + H]⁺ |
| 83 | F | H | H | 439.2, 441.2 |
| 84 | H | F | H | 439.2, 441.2 |
| 85 | H | H | F | 439.2, 441.3 |
| 86 | Cl | H | H | 455.0, 457.0 |
| 87 | H | Cl | H | 455.0, 457.0 |
| 88 | H | H | Cl | 455.2, 457.2 |
| 89 | Me | H | H | 435.0, 437.1 |
| 90 | H | Me | H | 435.0, 437.1 |
| 91 | H | H | Me | 435.0, 437.0 |
| 92 | OMe | H | H | 451.2, 453.2 |
| 93 | H | OMe | H | 451.1, 453.2 |
| 94 | H | H | OMe | 451.2, 453.3 |
| 95 | H | H | 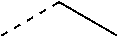 | 449.25 |
| 96 | H | H | 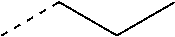 | 463.16 |
| 97 | H | H | 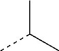 | 463.16 |

TABLE 8

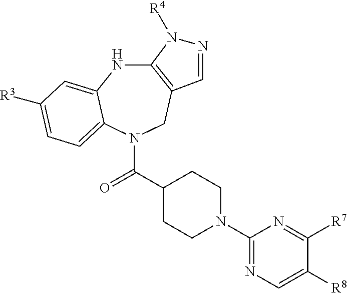

| | | | | | (ESI⁺) |
|---|---|---|---|---|---|
| Example | R³ | R⁴ | R⁷ | R⁸ | [M + H]⁺ |
| 98 | H | Me | H | H | 390.2 |
| 99 | H | 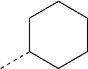 | CF₃ | H | 526.1 |
| 100 | F | Me | H | H | 408.2 |
| 101 | F | Me | CF₃ | H | 490.2 |
| 102 | Me | Me | H | H | 404.2 |
| 103 | Cl | Me | H | H | 426.2 |
| 104 | Cl | 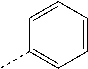 | H | H | 486.2 |
| 105 | Cl | 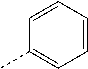 | H | 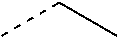 | 514.2, 516.2 |

TABLE 9

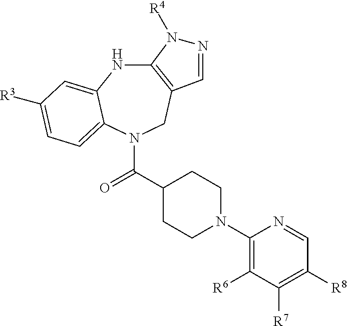

| | | | | | | (ESI⁺) |
|---|---|---|---|---|---|---|
| Example | R³ | R⁴ | R⁶ | R⁷ | R⁸ | [M + H]⁺ |
| 106 | H | Me | H | H | H | 389.1 |
| 107 | F | 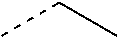 | H | H | Me | 435.2 |
| 108 | F | 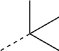 | H | H | Me | 463.2 |
| 109 | Me | Me | H | H | H | 403.2 |
| 110 | Me | Me | Me | H | H | 417.1 |
| 111 | Me | Me | H | Me | H | 417.2 |
| 112 | Me | Me | H | H | Me | 417.1 |
| 113 | Me | Me | OMe | H | H | 433.1 |
| 114 | Me | 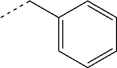 | H | H | Me | 493.3 |
| 115 | Cl | Me | H | H | H | 423.1, 425.1 |
| 116 | Cl | Me | Me | H | H | 437.2, 439.2 |
| 117 | Cl | Me | H | Me | H | 437.2 |
| 118 | Cl | Me | H | H | Me | 437.2, 439.2 |
| 119 | Cl | Me | OMe | H | H | 453.2 |
| 120 | Cl | 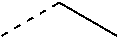 | Me | H | H | 451.1 |
| 121 | Cl | 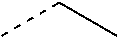 | H | Me | H | 451.1 |
| 122 | Cl | 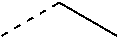 | H | H | Me | 451.2, 453.2 |
| 123 | Cl | 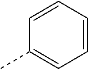 | H | H | Me | 499.1, 501.1 |

TABLE 10

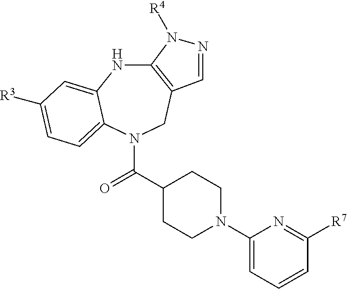

| | | | | (ESI⁺) |
|---|---|---|---|---|
| Example | R³ | R⁴ | R⁷ | [M + H]⁺ |
| 124 | Me | Me | Me | 417.2 |
| 125 | Cl | Me | Me | 437.2 |
| 126 | Cl | 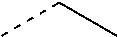 | Me | 451.1 |

TABLE 11

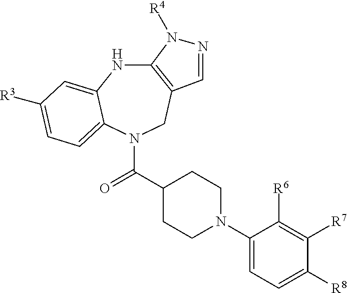

| | | | | | | (ESI⁺) |
|---|---|---|---|---|---|---|
| Example | R³ | R⁴ | R⁶ | R⁷ | R⁸ | [M + H]⁺ |
| 127 | Me | Me | F | H | H | 420.25 |
| 128 | Me | Me | H | Me | H | 416.2 |
| 129 | Me | Me | H | H | Me | 416.27 |
| 130 | Me | Me | H | OMe | H | 432.2 |
| 131 | Me | Me | H | H | OMe | 432.2 |
| 132 | Me | Me | H | H | 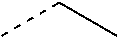 | 430.2 |
| 133 | Me | Me | H | H | CF₃ | 470.2 |
| 134 | Cl | Me | F | H | H | 440.2 |
| 135 | Cl | Me | H | Me | H | 436.2, 438.2 |
| 136 | Cl | Me | H | H | Me | 436.22 |
| 137 | Cl | Me | H | OMe | H | 452.2, 454.2 |
| 138 | Cl | Me | H | H | OMe | 452.1, 454.1 |
| 139 | Cl | Me | H | H | 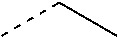 | 450.2, 452.2 |
| 140 | Cl | Me | H | H | CF₃ | 490.2 |

TABLE 12

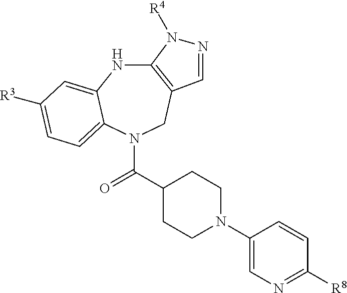

| | | | | (ESI⁺) |
|---|---|---|---|---|
| Example | R³ | R⁴ | R⁸ | [M + H]⁺ |
| 141 | Me | Me | Me | 417.2 |
| 142 | Cl | Me | Me | 437.2, 439.2 |

TABLE 13

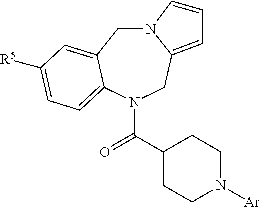

| | | | (ESI⁺) |
|---|---|---|---|
| Examples | R⁵ | Ar | [M + H]⁺ |
| 143 | H | 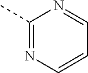 | 374.2 |
| 144 | H | 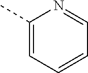 | (373.2 |
| 145 | H | 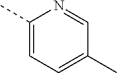 | 387.3 |
| 146 | H | 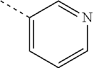 | 373.1 |
| 147 | H | 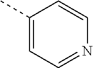 | 373.2 |
| 148 | H | 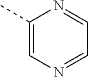 | 374.0 |
| 149 | H | 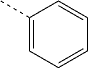 | 372.1 |
| 150 | F | 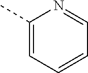 | 391.1 |
| 151 | F | 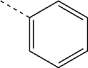 | 390.2 |
| 152 | Me | 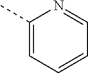 | 387.2 |
| 153 | Me | 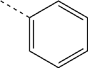 | 386.0 |

TABLE 14

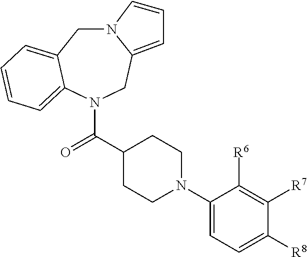

| | | | | (ESI⁺) |
|---|---|---|---|---|
| Example | R⁶ | R⁷ | R⁸ | [M + H]⁺ |
| 154 | F | H | H | 390.0 |
| 155 | H | F | H | 390.2 |
| 156 | H | H | F | 390.2 |
| 157 | Cl | H | H | 406.0, 408.1 |
| 158 | H | H | Cl | 406.2, 408.2 |
| 159 | Me | H | H | 386.2 |
| 160 | H | Me | H | 386.3 |
| 161 | H | H | Me | 386.2 |
| 162 | OMe | H | H | 402.3 |
| 163 | H | OMe | H | 402.3 |
| 164 | H | H | OMe | 402.3 |
| 165 | H | H | 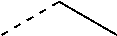 | 400.2 |
| 166 | H | H | 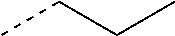 | 414.3 |
| 167 | H | H | 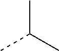 | 414.3 |
| 168 | H | 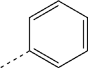 | H | 448.3 |
| 169 | H | H | CF₃ | 440.2 |
| 170 | H | CN | H | 397.2 |
| 171 | H | H | CN | 397.3 |
| 172 | NO₂ | H | H | 401.2 |
| 173 | H | NO₂ | H | 417.2 |
| 174 | H | H | NO₂ | 417.2 |

### Example 175

### [1-(2-Amino-phenyl)-piperidin-4-yl]-(5H,11H-benzo[e]pyrrolo[1,2-a][1,4]diazepin-10-yl)-methanone

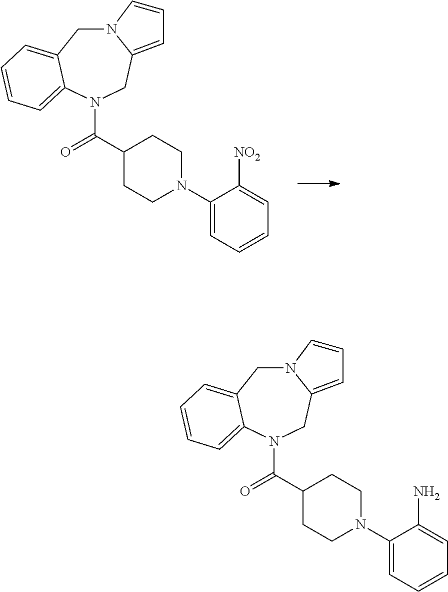

[1-(2-nitro-phenyl)-piperidin-4-yl]-(5H,11H-benzo[e]pyrrolo[1,2-a][1,4]diazepin-10-yl)-methanone (1.0 g, 2.4 mmol) and 10% Pd/C (100 mg) were stirred in ethanol (20 ml) and DMF (5 ml) under an atmosphere of H₂ for 3 h. The mixture was filtered through Celite™ and solvent removed in vacuo. The residue was purified by flash chromatography on silica (eluant: EtOAc/petroleum ether 40/60 v/v) to yield the title compound (814 mg, 2.1 mmol, 88%) as a pale orange solid.

¹H NMR δ 1.89-2.03 (4H, m), 2.36-2.43 (3H, m), 3.02-3.16 (2H, m), 3.95 (2H, brs), 4.10-4.18 (1H, m), 4.71 (1H, d, J=13.8 Hz), 5.27 (1H, d, J=13.8 Hz), 5.98-6.06 (3H, m), 6.58-6.70 (4H, m), 6.82-6.88 (2H, m), 7.25-7.44 (3H, m).

(ESP⁺): [M+H]⁺=387.25

The following example was prepared by methods analogous to the above:

TABLE 15

| 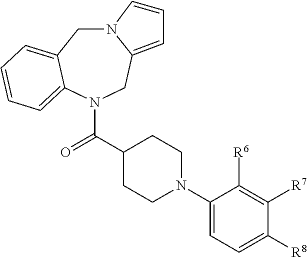 | | | | |
|---|---|---|---|---|
| | | | | (ESI⁺) |
| Example | R⁶ | R⁷ | R⁸ | [M + H]⁺ |
| 176 | H | H | NH₂ | 387.3 |

### Example 177

### [1-(2-Aminomethyl-phenyl)-piperidin-4-yl]-(5H,11H-benzo[e]pyrrolo[1,2-a][1,4]diazepin-10-yl)-methanone

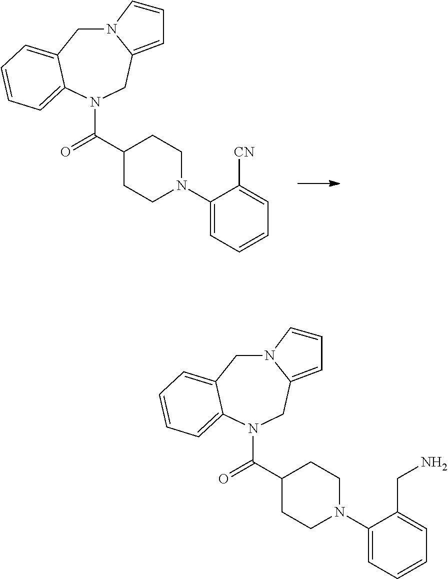

Cobalt (II) chloride hexahydrate (240 mg, 1.00 mmol) was added to a solution of 2-[4-(5H,11H-Benzo[e]pyrrolo[1,2-a][1,4]diazepine-10-carbonyl)-piperidin-1-yl]-benzonitrile (200 mg, 0.50 mmol) in MeOH (10 mL) at RT, and the mixture was stirred for 10 min before being cooled to 0° C. Sodium borohydride (191 mg, 0.5 mmol) was added portionwise, and the mixture was then stirred at 0° C. for 15 min and at RT for a further 48 h. before being purified by flash chromatography on silica (eluant: with DCM/MeOH/25% aq. ammonia 90/10/2 v/v/v) to yield the title compound (140 mg, 0.30 mmol, 69%) as a white solid.

¹H NMR δ 1.48-1.52 (1H, m), 1.89-1.99 (3H, m), 2.36-2.49 (3H, m), 2.98-3.11 (4H, m), 3.93 (2H, s), 4.13 (1H, d, J=16.1 Hz), 4.69 (1H, d, J=13.6 Hz), 5.26 (1H, d, J=13.6 Hz), 5.93-5.97 (2H, m), 6.05 (1H, t, J=3.0 Hz), 6.57 (1H, s), 7.04-7.43 (8H, m).

(ESI⁺): [M+H]⁺=401.2

The following example was prepared by methods analogous to the above:

TABLE 16

| 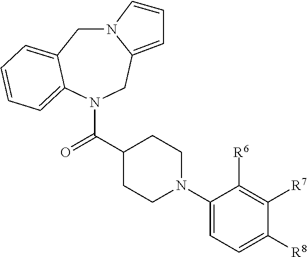 | | | | |
|---|---|---|---|---|
| | | | | (ESI⁺) |
| Example | R⁶ | R⁷ | R⁸ | [M + H]⁺ |
| 178 | H | H | CH₂NH₂ | 401.2 |

TABLE 17
NMR Data for Examples

| Example | ¹H NMR |
|---|---|
| 6 | 1.05-1.17 (1H, m), 1.17-1.42 (1H, m), 1.75-2.00 (2H, m), 2.30-2.50 (1H, |
| | m), 2.42 (3H, s), 2.60-2.82 (3H, m), 2.82-3.00 (1H, m), 3.22-3.42 (1H, |
| | m), 4.46 (1H, d, J = 13.4 Hz), 4.65-4.82 (2H, m), 6.38 (1H, t, J = 4.7 Hz), |
| | 7.12-7.28 (2H, m), 7.30-7.44 (1H, m) 8.15-(1H, d, J = 7.4 Hz), 8.20 (1H, d, |
| | J = 4.7 Hz) |
| 7 | 1.04-1.16 (1H, m), 1.23-1.40 (1H, m), 1.71-2.00 (2H, m), 2.44 (3H, s), |
| | 2.45-2.53 (1H, m), 2.66-2.98 (4H, m), 3.22-3.41 (1H, m), 4.47 (1H, d, |
| | J = 13.4 Hz), 4.68-4.82 (2H, m), 6.39 (1H, t, J = 4.7 Hz), 7.07-7.22 (2H, m), |
| | 8.20-8.25 (3H, m), 9.29 (1H, s) |
| 8 | 1.02-1.13 (1H, m), 1.21-1.43 (1H, m), 1.77-2.02 (2H, m), 2.30-2.32 (1H, |
| | m), 2.39 (3H, s), 2.54-2.79 (3H, m), 2.80-2.97 (1H, m), 3.21-3.40 (1H, |
| | m), 3.88-4.00 (1H, m) 4.21-4.35 (1H, m), 4.70 (1H, dd, J = 5.2 Hz) |
| | 6.48-6.53 (2H, m), 7.18-7.20 (2H, m), 7.30-7.41 (2H, m), 8.07 (1H, d, |
| | J = 3.9 Hz), 8.19 (1H, s), 10.40 (1H, s) |
| 9 | (DMSO-d6) 0.92-0.95 (1H, m), 1.08-1.18 (1H, m), 1.62-1.79 (2H, m), |
| | 2.28-2.35 (1H, m), 2.59-2.74 (4H, m), 3.02-3.10 (1H, m), 3.27-3.31 (4H, |
| | m), 3.69-3.72 (4H, m), 4.01 (1H, d, J = 13.4 Hz), 4.28 (1H, d, J = 13.2 Hz), |
| | 4.54-4.58 (1H, m), 6.51-6.59 (1H, m), 6.72 (1H, d, J = 8.6 Hz), |
| | 7.18-7.21 (1H, m), 7.34 (2H, d, J = 6.7 Hz), 7.42-7.46 (1H, m), 7.98-8.07 (2H, m), |
| | 11.17 (1H, s). |
| 10 | (DMSO-d6): 0.99 (1H, d, J = 12.2 Hz), 1.29-1.41 (1H, m), 1.79-1.90 (2H, |
| | m), 2.14 (3H, s), 2.14-2.23 (1H, m), 2.47-2.75 (4H, m), 3.04-3.14 (2H, |
| | m), 3.23-3.38 (5H, m), 3.70 (4H, t, J = 4.7 Hz), 4.59 (1H, d, J = 10.9 Hz), |
| | 6.84 (1H, dd, J = 4.7, 7.5 Hz), 7.16-7.20 (1H, m), 7.31-7.36 (2H, m), |
| | 7.42 (1H, dd, =1.4, 7.5 Hz), 8.00 (1H, dd, J = 1.8, 4.7 Hz), 8.06 (1H, brs), |
| | 11.15 (1H, brs). |
| 11 | (DMSO-d6) 0.92-0.95 (1H, m), 1.08-1.18 (1H, m), 1.61-1.72 (1H, m), |
| | 1.76-1.79 (1H, m), 2.16 (3H, s), 2.30-2.35 (1H, m), 2.61-2.76 (4H, m), |
| | 3.01-3.10 (1H, m), 3.31-3.36 (4H, m), 3.71-3.73 (4H, m), 4.02 (1H, d, J = 13.2 Hz), |
| | 4.26 (1H, d, J = 13.3 Hz), 4.54-4.59 (1H, m), 6.42 (1H, d, J = 5.1 Hz), |
| | 6.59 (1H, s), 7.27-7.40 (3H, m), 7.88 (1H, d, J = 5.1 Hz), |
| | 7.94-8.01 (1H, m). |
| 12 | (DMSO-d6) 0.97-1.06 (1H, m), 1.15-1.25 (1H, m), 1.69-1.84 (2H, m), |
| | 2.16 (3H, s), 2.30-2.37 (1H, m), 2.60-2.68 (1H, m), 2.70-2.82 (3H, m), |
| | 3.07-3.16 (1H, m), 3.34-3.38 (4H, m), 3.76-3.79 (4H, m), 4.01 (1H, d, J = 13.3 Hz), |
| | 4.27 (1H, d, J = 12.9 Hz), 4.60-4.65 (1H, m), 6.72 (1H, d, |
| | J = 8.7 Hz), 7.24-7.28 (1H, m), 7.35-7.43 (3H, m), 7.94 (1H, d, J = 1.8 Hz), |
| | 7.99-8.06 (1H, m), 11.26 (1H, d, J = 5.3 Hz). |
| 13 | (DMSO-d6): d 0.92 (1H, d, J = 12.2 Hz), 1.10-1.16 (1H, m), 1.60-1.71 (1H, |
| | m), 1.77 (1H, d, J = 12.2 Hz), 2.33-2.40 (1H, m), 2.61-2.76 (4H, m), |
| | 3.01-3.13 (1H, m), 3.31-3.34 (4H, m), 3.69 (4H, t, J = 4.4 Hz), 4.00 (1H, d, |
| | J = 12.7 Hz), 4.28 (1H, d, J = 12.7 Hz), 4.51-4.57 (1H, m), 6.54 (1H, dd, J = −5.2, |
| | 7.1 Hz), 6.72 (1H, d, J = 8.7 Hz), 6.99 (1H, dt, J = 2.9, 8.2 Hz), |
| | 7.36-7.40 (1H, m), 7.41-7.46 (1H, m), 7.68-7.76 (1H, m), 8.03 (1H, dd, J = 1.2, |
| | 4.9 Hz), 11.24 (1H, s) |
| 14 | (DMSO-d6): 0.98 (1H, d, J = 10.9 Hz), 1.28-1.38 (1H, m), 1.81-1.91 (2H, |
| | m), 2.14 (3H, s), 2.21 (1H, dt, J = 1.9, 12.2 Hz), 2.54 (2H, dd, J = 3.6, |
| | 12.2 Hz), 2.67-2.76 (2H, m), 3.05-3.16 (2H, m), 3.25 (2H, t, J = 4.2 Hz), |
| | 3.30-3.39 (3H, m), 3.69 (4H, t, J = 5.2 Hz), 4.58 (1H, dd, J = 5.2, 12.2 Hz), |
| | 6.84 (1H, dd, J = 5.0, 6.7 Hz), 6.98 (1H, dt, J = 3.3, 7.9 Hz), 7.36-7.43 (2H, |
| | m), 7.71 (1H, dd, J = 2.8, 11.3 Hz), 8.01 (1H, d, J = 5.0 Hz), 11.24 (1H, s). |
| 15 | (DMSO-d6): 0.90 (1H, d, J = 12.1 Hz), 1.04-1.15 (1H, m), 1.59-1.69 (1H, |
| | m), 1.75 (1H, d, J = 12.1 Hz), 2.15 (3H, s), 2.36 (1H, dt, J = 2.6, 13.1 Hz), |
| | 2.58-2.75 (4H, m), 3.01-3.10 (1H, m), 3.25 (2H, t, J = 5.5 Hz), |
| | 3.30-3.35 (2H, m), 3.70 (4H, t, J = 4.9 Hz), 4.00 (1H, d, J = 12.5 Hz), 4.28 (1H, d, |
| | J = 12.5 Hz), 4.55 (1H, dd, J = 5.5, 13.1 Hz), 6.40 (1H, d, J = 5.2 Hz), |
| | 6.55 (1H, s), 6.99 (1H, dt, J = 3.1, 7.8 Hz), 7.38 (1H, dd, J = 5.2, 8.3 Hz), |
| | 7.72 (1H, dd, J = 3.1, 11.0 Hz), 7.89 (1H, d, J = 5.7 Hz), 11.23 (1H, s). |
| 16 | (DMSO-d6): 0.90 (1H, d, J = 11.5 Hz), 1.05-1.15 (1H, m), 1.60-1.70 (1H, |
| | m), 1.73-1.78 (1H, m), 2.09 (3H, s), 2.224-2.36 (1H, m), 2.54-2.76 (4H, |
| | m), 2.96-3.11 (1H, m), 3.21-3.27 (4H, m), 3.69 (4H, t, J = 4.5 Hz), |
| | 3.93 (1H, d, J = 12.7 Hz), 4.20 (1H, d, J = 12.7 Hz), 4.50-4.59 (1H, m), 6.65 (1H, |
| | d, J = 8.7 Hz), 6.98 (1H, dt, J = 3.0, 8.4 Hz), 7.28 (1H, dd, J = 2.2, 8.7 Hz), |
| | 7.37 (1H, dd, J = 5.5, 8.4 Hz), 7.71 (1H, dd, J = 2.8, 10.7 Hz), 7.8 (1H, d, |
| | J = 2.1 Hz), 11.23 (1H, s) |
| 17 | (DMSO-d6) 1.01-1.05 (1H, m), 1.26-1.31 (3H, m), 1.35-1.45 (1H, m), |
| | 1.90-1.98 (2H, m), 2.20 (3H, s), 2.25-2.30 (1H, m), 2.43 (3H, s), |
| | 2.60-2.89 (6H, m), 3.14-3.23 (2H, m), 3.41-3.44 (1H, m), 4.63-4.68 (1H, m), |
| | 6.89-6.92 (1H, m), 7.08 (1H, d, J = 7.5 Hz), 7.28 (1H, d, J = 7.7 Hz), |
| | 7.48 (1H, dd, J = 1.0, 7.4 Hz), 8.01 (1H, s), 8.07 (1H, dd, J = 1.6, 4.8 Hz), |
| | 11.83 (1H, s). |
| 18 | (DMSO-d6) 0.94-0.97 (1H, m), 1.12-1.22 (1H, m), 1.30 (3H, t, J = 7.6 Hz), |
| | 1.66-1.77 (1H, m), 1.80-1.84 (1H, m), 2.22 (3H, s), 2.36-2.41 (1H, m), |
| | 2.43 (3H, s), 2.64-2.83 (6H, m), 3.10-3.19 (1H, m), 4.05 (1H, d, |
| | J = 13.2 Hz), 4.34 (1H, d, J = 13.3 Hz), 4.60-4.64 (1H, m), 6.41 (1H, d, |
| | J = 5.0 Hz), 6.61 (1H, s), 7.09 (1H, d, J = 7.5 Hz), 7.28 (1H, d, J = 7.9 Hz), |
| | 7.95 (1H, d, J = 5.0 Hz), 7.98 (1H, s), 11.84 (1H, s). |
| 19 | 1.05-1.08 (1H, m), 1.29 (3H, t, J = 7.7 Hz), 1.32-1.38 (1H, m), |
| | 1.75-1.78 (1H, m), 1.85-1.95 (1H, m), 2.07 (3H, s), 2.27-2.31 (1H, m), 2.35 (3H, s), |
| | 2.56-2.86 (6H, m), 3.25-3.35 (1H, m), 3.84 (1H, d, J = 13.0 Hz), 4.15 (1H, |
| | d, J = 13.0 Hz), 4.68 (1H, dd, J = 4.9, 13.3 Hz), 6.41 (1H, d, J = 8.6 Hz), |
| | 6.93-6.96 (1H, m), 7.00-7.02 (1H, m), 7.16 (1H, dd, J = 2.3, 8.7 Hz), 7.86 (1H, |
| | d, J = 2.2 Hz), 7.93 (1H, s), 9.15 (1H, br s). |
| 20 | 0.93 (3H, t, J = 7.3 Hz), 1.05-1.08 (1H, m), 1.17-1.46 (3H, m), |
| | 1.68-1.79 (3H, m), 1.84-1.94 (1H, m), 2.35 (3H, s), 2.36-2.40 (1H, m), |
| | 2.61-2.72 (4H, m), 2.79-2.86 (1H, m), 3.26-3.35 (1H, m), 3.91 (1H, d, J = 13.2 Hz), |
| | 4.23 (1H, d, J = 13.1 Hz), 4.65-4.69 (1H, m), 6.45-6.49 (2H, m), |
| | 6.93-7.03 (2H, m), 7.31-7.35 (1H, m), 7.91 (1H, s) 8.02-8.04 (1H, m). |
| 21 | (DMSO-d6) 0.97 (3H, t, J = 7.4 Hz), 1.03-1.04 (1H, m), 1.34-1.45 (1H, m), |
| | 1.70-1.79 (2H, m), 1.89-1.98 (2H, m), 2.20 (3H, s), 2.25-2.30 (1H, m), |
| | 2.43 (3H, s), 2.60-2.68 (4H, m), 2.77-2.86 (2H, m), 3.15-3.24 (2H, m), |
| | 3.41-3.45 (1H, m), 4.60-4.68 (1H, m), 6.89-6.92 (1H, m), 7.06-7.13 (1H, |
| | m), 7.25-7.34 (1H, m), 7.48 (1H, d, J = 7.2 Hz), 8.01 (1H, d, J = 1.2 Hz), |
| | 8.07 (1H, dd, J = 1.4, 4.7 Hz), 11.82 (1H, s). |
| 22 | 0.77-0.83 (1H, m), 0.94 (3H, t, J = 7.3 Hz), 1.04-1.07 (1H, m), |
| | 1.26-1.37 (1H, m), 1.68-1.77 (2H, m), 1.84-1.94 (1H, m), 2.13 (3H, s), 2.32-2.34 |
| | and 2.38-2.39 (1H, m), 2.35 (3H, s), 2.60-2.71 (5H, m), 2.79-2.86 (1H, |
| | m), 3.27-3.36 (1H, m), 3.90 (1H, d, J = 13.3 Hz), 4.21 (1H, d, J = 13.2 Hz), |
| | 4.67-4.71 (1H, m), 6.30-6.32 (2H, m), 6.93-6.97 (1H, m), 7.00-7.02 (1H, |
| | m), 7.89-8.03 (2H, m). |
| 23 | 0.92 (3H, t, J = 7.3 Hz), 1.04-1.07 (1H, m), 1.17-1.46 (3H, m), |
| | 1.67-1.78 (3H, m), 1.84-1.94 (1H, m), 2.08 (3H, s), 2.29-2.33 (1H, m), 2.35 (3H, s), |
| | 2.57-2.71 (4H, m), 2.78-2.86 (1H, m), 3.25-3.34 (1H, m), 3.83 (1H, d, |
| | J = 13.0 Hz), 4.14 (1H, d, J = 13.1 Hz), 4.67 (1H, dd, J = 5.0, 13.3 Hz), |
| | 6.43 (1H, d, J = 8.7 Hz), 6.93-7.03 (2H, m), 7.16-7.19 (1H, m), 7.86 (1H, d, |
| | J = 2.2 Hz), 7.90 (1H, s). |
| 24 | (DMSO-d6) 0.92-0.98 (5H, m), 1.13-1.23 (1H, m), 1.67-1.77 (1H, m), |
| | 1.82-1.85 (1H, m), 1.96-2.03 (1H, m), 2.38-2.41 (1H, m), 2.43 (3H, s), |
| | 2.66-2.69 (1H, m), 2.72-2.80 (3H, m), 3.07-3.16 (1H, m), 4.07 (1H, d, |
| | J = 13.4 Hz), 4.35 (1H, d, J = 13.3 Hz), 4.58-4.63 (1H, m), 6.60-6.63 (1H, |
| | m), 6.79 (1H, d, J = 8.7 Hz), 7.09 (1H, d, J = 7.6 Hz), 7.29 (1H, d, J = 7.8 Hz), |
| | 7.49-7.53 (1H, m), 7.92 (1H, s), 8.09-8.11 (1H, m), 11.84 (1H, s). |
| 25 | (DMSO-d6) 0.90-1.00 (5H, m), 1.13-1.23 (1H, m), 1.67-1.84 (2H, m), |
| | 1.94-2.03 (1H, m), 2.16 (3H, s), 2.32-2.39 (1H, m), 2.43 (3H, s), |
| | 2.61-2.68 (1H, m), 2.70-2.80 (3H, m), 3.07-3.16 (1H, m), 4.00 (1H, d, |
| | J = 13.2 Hz), 4.28 (1H, d, J = 13.2 Hz), 4.58-4.63 (1H, m), 6.72 (1H, d, |
| | J = 8.7 Hz), 7.09 (1H, d, J = 7.2 Hz), 7.28 (1H, d, J = 7.8 Hz), 7.36 (1H, dd, |
| | J = 2.4, 8.7 Hz), 7.94 (2H, d, J = 2.4 Hz), 11.85 (1H, s). |
| 26 | (DMSO-d6) 0.90-0.94 (4H, m), 1.07-1.17 (1H, m), 1.32-1.40 (2H, m), |
| | 1.62-1.72 (3H, m), 1.77-1.79 (1H, m), 2.32-2.36 (1H, m), 2.38 (3H, s), |
| | 2.60-2.77 (6H, m), 3.05-3.14 (1H, m), 4.00 (1H, d, J = 13.4 Hz), 4.29 (1H, |
| | d, J = 13.4 Hz), 4.54-4.58 (1H, m), 6.54-6.57 (1H, m), 6.73 (1H, d, |
| | J = 8.8 Hz), 7.04 (1H, d, J = 7.4 Hz), 7.23 (1H, d, J = 7.8 Hz), 7.43-7.48 (1H, |
| | m), 7.93 (1H, s), 8.03-8.05 (1H, m), 11.80 (1H, s). |
| 27 | (DMSO-d6) 0.87 (1H, s), 0.92 (3H, t, J = 7.4 Hz), 1.07-1.17 (1H, m), |
| | 1.30-1.39 (2H, m), 1.61-1.72 (3H, m), 1.75-1.77 (1H, m), 2.11 (3H, s), |
| | 2.27-2.34 (1H, m), 2.38 (3H, s), 2.55-2.84 (6H, m), 3.05-3.14 (1H, m), |
| | 3.93 (1H, d, J = 13.3 Hz), 4.21 (1H, d, J = 13.2 Hz), 4.53-4.58 (1H, m), |
| | 6.63-6.71 (1H, m), 7.03 (1H, d, J = 7.5 Hz), 7.22 (1H, d, J = 7.7 Hz), 7.30 (1H, dd, |
| | J = 2.3, 8.7 Hz), 7.88 (1H, d, J = 2.3 Hz), 7.94 (1H, s), 11.78 (1H, s). |
| 28 | (DMSO-d6) 0.88 (1H, s), 0.91-0.93 (6H, m), 1.07-1.16 (1H, m), |
| | 1.62-1.72 (1H, m), 1.76-1.79 (1H, m), 1.99-2.09 (1H, m), 2.32-2.36 (1H, m), |
| | 2.38 (3H, s), 2.48 (2H, d, J = 7.2 Hz), 2.61-2.78 (4H, m), 3.07-3.16 (1H, |
| | m), 3.99 (1H, d, J = 13.2 Hz), 4.28 (1H, d, J = 13.3 Hz), 4.53-4.58 (1H, m), |
| | 6.54-6.57 (1H, m), 6.72 (1H, d, J = 8.6 Hz), 7.04 (1H, d, J = 7.5 Hz), |
| | 7.23 (1H, d, J = 7.5 Hz), 7.43-7.48 (1H, m), 7.94 (1H, s), 8.03-8.05 (1H, m), |
| | 11.78 (1H, s). |
| 29 | (DMSO-d6) 0.92 (1H, s), 0.96-0.98 (6H, m), 1.12-1.23 (1H, m), |
| | 1.67-1.82 (2H, m), 2.04-2.13 (1H, m), 2.16 (3H, s), 2.28-2.39 (1H, m), |
| | 2.43 (3H, s), 2.54 (2H, d, J = 7.3 Hz), 2.61-2.83 (4H, m), 3.12-3.21 (1H, m), |
| | 3.97 (1H, d, J = 13.3 Hz), 4.26 (1H, d, J = 13.2 Hz), 4.59-4.63 (1H, m), |
| | 6.71 (1H, d, J = 8.7 Hz), 7.09 (1H, d, J = 7.3 Hz), 7.28 (1H, d, J = 7.8 Hz), |
| | 7.36 (1H, dd, J = 2.3, 8.7 Hz), 7.93-7.99 (2H, m), 11.85 (1H, s). |
| 30 | 1.10-1.13 (1H, m), 1.32-1.42 (1H, m), 1.69-1.77 (3H, m), 1.84-1.93 (1H, |
| | m), 2.07 (3H, s), 2.27 (3H, s), 2.33 (3H, s), 2.47 (4H, t, J = 4.9 Hz), |
| | 2.54-2.67 (2H, m), 2.76-2.81 (1H, m), 3.17-3.26 (1H, m), 3.37 (4H, t, |
| | J = 4.7 Hz), 3.84 (1H, d, J = 11.8 Hz), 4.17 (1H, d, J = 11.4 Hz), 4.61 (1H, d, J |
| | 8.4 Hz), 6.41 (1H, d, J = 8.6 Hz), 6.88-6.90 (1H, m), 6.97-6.99 (1H, m), |
| | 7.16 (1H, dd, J = 2.2, 8.6 Hz), 7.86 (1H, d, J = 1.7 Hz), 7.93 (1H, s), |
| | 8.91 (1H, s). |
| 31 | (DMSO-d6): d 0.93 (1H, d, J = 11.8 Hz), 1.15-1.18 (1H, m), |
| | 1.61-1.71 (1H, m), 1.75 (1H, d, J = 10.5 Hz), 2.31-2.33 (1H, m), 2.34 (3H, s), |
| | 2.58-2.75 (4H, m), 2.97-3.11 (1H, m), 3.22-3.29 (4H, m), 3.69 (4H, t, |
| | J = 4.5 Hz), 4.01 (1H, d, J = 12.9 Hz), 4.27 (1H, d, J = 12.9 Hz), 4.51-4.57 (1H, |
| | m), 6.53 (1H, dd, J = 5.2, 7.0 Hz), 6.71 (1H, d, J = 8.6 Hz), 6.98 (1H, d, |
| | J = 7.7 Hz), 7.17 (1H, d, J = 7.7 Hz), 7.42-7.46 (1H, m), 7.86 (1H, s), |
| | 8.03 (1H, dd, J = 1.2, 4.9 Hz), 11.10 (1H, s) |
| 32 | 1.00-1.03 (1H, m), 1.07-1.12 (1H, m), 1.17-1.28 (1H, m), 1.65-1.68 (1H, |
| | m), 1.75-1.82 (1H, m), 2.07 (3H, s), 2.25 (3H, s), 2.29-2.35 (1H, m), |
| | 2.49-2.72 (3H, m), 3.09-3.18 (1H, m), 3.27-3.29 (4H, m), 3.54-3.68 (5H, |
| | m), 3.81 (1H, d, J = 13.1 Hz), 4.09 (1H |
| | H, d, J = 13.3 Hz), 4.52-4.56 (1H, m), 6.25 (1H, s), 6.26 (1H, s), |
| | 6.83-6.86 (1H, m), 6.90-6.92 (1H, m), 7.66 (1H, s), 7.79 (1H, d, J = 5.6 Hz). |
| 33 | (DMSO-d6): 0.94 (1H, d, J = 12.9 Hz), 1.08-1.18 (1H, m), 1.61-1.72 (1H, |
| | m), 1.77 (1H, d, J = 12.9 Hz), 2.32-2.37 (4H, m), 2.61-2.73 (4H, m), |
| | 3.00-3.09 (2H, m), 3.29-3.36 (3H, m), 3.72 (4H, t, J = 5.1 Hz), 3.74 (3H, s), |
| | 4.00 (1H, d, J = 13.1 Hz), 4.25 (1H, d, J = 13.1 Hz), 4.55 (1H, dd, J = 5.7, 12.0 Hz), |
| | 6.21-6.24 (2H, m), 7.06 (1H, d, J = 5.5 Hz), 7.26 (1H, d, J = 6.6 Hz), |
| | 7.75 (1H, brs), 7.85 (1H, d, J = 6.6 Hz), 11.50 (1H, brs). |
| 34 | (DMSO-d6): 0.93 (1H, d, J = 12.5 Hz), 1.11-1.23 (1H, m), 1.63-1.77 (2H, |
| | m), 2.25 (1H, dt, J = 1.9, 11.9 Hz), 2.36 (3H, s), 2.55 (1H, dt, J = 2.5, |
| | 12.5 Hz), 2.61-2.73 (3H, m), 3.00-3.09 (2H, m), 3.24-3.46 (3H, m), |
| | 3.68 (3H, s), 3.69-3.73 (4H, m), 3.87 (1H, d, J = 12.5 Hz), 4.11 (1H, d, |
| | J = 13.4 Hz), 4.54 (1H, dd, J = 5.0 Hz), 6.72 (1H, d, J = 8.9 Hz), 7.00-7.05 (1H, |
| | m), 7.18 (1H, dd, J = 9.8 Hz), 7.20-7.26 (1H, m), 7.72-7.77 (1H, m), |
| | 7.81 (1H, d, J = 3.1 Hz), 11.20 (1H, brs). |
| 35 | (DMSO-d6) d 0.98 (1H, d, J = 11.3 Hz), 1.07-1.18 (1H, m), 1.59-1.71 (1H, |
| | m), 1.81 (1H, d, J = 11.3 Hz), 2.35 (3H, s), 2.47-2.51 (1H, m), |
| | 2.66-2.82 (4H, m), 2.92-3.05 (1H, m), 3.25-3.31 (4H, m), 3.70 (4H, t, J = 4.9 Hz), |
| | 4.12 (1H, d, J = 13.1 Hz), 4.39 (1H, d, J = 13.1 Hz), 4.52-4.56 (1H, m), |
| | 6.66 (1H, d, J = 9.2 Hz), 6.98 (1H, d, J = 7.6 Hz), 7.18-7.20 (1H, m), 7.70 (1H, dd, |
| | J = 2.4, 9.2 Hz), 7.86 (1H, s), 8.33 (1H, s), 11.10 (1H, s). |
| 36 | (DMSO-d6) 0.97-1.10 (2H, m), 1.59-1.69 (1H, m), 1.80-1.83 (1H, m), |
| | 2.35 (3H, s), 2.66-2.84 (4H, m), 3.16 (2H, d, J = 5.4 Hz), 3.25-3.29 (4H, m), |
| | 3.69-3.71 (4H, m), 4.13 (1H, d, J = 12.7 Hz), 4.41 (1H, d, J = 12.8 Hz), |
| | 4.53-4.56 (1H, m), 6.83 (1H, d, J = 9.1 Hz), 6.98 (1H, d, J = 7.7 Hz), 7.19 (1H, d, |
| | J = 7.6 Hz), 7.76 (1H, dd, J = 2.3, 9.1 Hz), 7.86 (1H, s), 8.41 (1H, d, |
| | J = 2.1 Hz), 11.10 (1H, s). |
| 37 | (DMSO-d6): d 0.96 (1H, d, J = 12.1 Hz), 1.29-1.40 (1H, m), 1.66-1.88 (6H, |
| | m), 2.13 (3H, s), 2.18-2.22 (1H, m), 2.36 (3H, s), 2.47-2.53 (1H, m), |
| | 2.55-2.63 (1H, m), 2.70-2.81 (2H, m), 2.85-2.93 (1H, m), 3.11 (2H, d, |
| | J = 12.1 Hz), 3.34-3.45 (3H, m), 3.89-3.93 (2H, m), 4.60 (1H, dd, J = 4.7, |
| | 13.1 Hz), 6.84 (1H, dd, J = 5.0, 7.0 Hz), 7.01 (1H, dd, J = 1.8, 8.1 Hz), |
| | 7.20 (1H, d, J = 8.1 Hz), 7.41 (1H, d, J = 7.0 Hz), 7.96 (1H, d, J = 1.3 Hz), 8.00 (1H, |
| | dd, J = 1.3, 5.0 Hz), 11.79 (1H, s). |
| 38 | (DMSO-d6): d 0.89 (1H, d, J = 12.1 Hz), 1.07-1.17 (1H, m), 1.59-1.87 (6H, |
| | m), 2.15 (3H, s), 2.29-2.37 (1H, m), 2.36 (3H, s), 2.61 (1H, dt, J = 2.1, |
| | 12.5 Hz), 2.66-2.78 (3H, m), 2.86-2.94 (1H, m), 3.03-3.12 (1H, m), |
| | 3.39-3.46 (2H, m), 3.89-3.94 (2H, m), 3.99 (1H, d, J = 13.1 Hz), 4.27 (1H, d, |
| | J = 13.1 Hz), 4.55 (1H, dd, J = 4.1, 13.1 Hz), 6.39 (1H, d, J = 5.0 Hz), |
| | 6.54 (1H, s), 7.02 (1H, d, J = 7.7 Hz), 7.21 (1H, d, J = 7.7 Hz), 7.88 (1H, d, |
| | J = 5.0 Hz), 7.95 (1H, s), 11.79 (1H, s). |
| 39 | (DMSO-d6): d 0.89 (1H, d, J = 12.1 Hz), 1.07-1.17 (1H, m), 1.60-1.88 (6H, |
| | m), 2.09 (3H, s), 2.29 (1H, t, J = 12.5 Hz), 2.36 (3H, s), 2.58 (1H, t, |
| | J = 12.5 Hz), 2.64-2.79 (3H, m), 2.85-2.94 (1H, m), 3.01-3.13 (1H, m), |
| | 3.42 (2H, t, J = 11.4 Hz), 3.90-3.94 (3H, m), 4.20 (1H, d, J = 12.5 Hz), 4.56 (1H, |
| | d, J = 4.0, 13.2 Hz), 6.64 (1H, d, J = 8.8 Hz), 7.01 (1H, d, J = 7.9 Hz), |
| | 7.19 (1H, d, J = 7.9 Hz), 7.28 (1H, dd, J = 1.7, 8.8 Hz), 7.87 (1H, d, J = 1.7 Hz), |
| | 7.96 (1H, s), 11.78 (1H, s). |
| 40 | 1.09 (1H, d, J = 12.6 Hz), 1.48-1.53 (1H, m), 1.78-2.08 (6H, m), 2.24 (1H, |
| | t, J = 11.3 Hz), 2.35 (3H, s), 2.55-2.71 (3H, m), 2.84 (1H, t, J = 12.6 Hz), |
| | 3.01 (1H, t, J = 11.3 Hz), 3.30-3.40 (1H, m), 3.47-3.51 (2H, m), 3.62 (1H, |
| | d, J = 12.7 Hz), 3.70 (3H, s), 3.86 (1H, d, J = 12.7 Hz), 3.99-4.04 (2H, m), |
| | 4.70 (1H, dd, J = 5.2, 13.1 Hz), 6.69 (1H, dd, J = 4.7, 8.0 Hz), 6.89 (1H, d, |
| | J = 8.0 Hz), 6.95 (1H, d, J = 8.0 Hz), 7.03 (1H, d, J = 8.0 Hz), 7.71 (1H, dd, |
| | J = 1.5, 4.7 Hz), 8.01 (1H, s), 9.05 (1H, s). |
| 41 | — |
| 42 | (DMSO-d6): d 0.89 (1H, d, J = 12.1 Hz), 1.10-1.22 (1H, m), 1.62-1.88 (6H, |
| | m), 2.25 (1H, dt, J = 2.3, 12.7 Hz), 2.35 (3H, s), 2.54 (1H, dt, J = 2.3, |
| | 12.7 Hz), 2.61-2.79 (3H, m), 2.85-2.93 (1H, m), 3.02-3.13 (1H, m), |
| | 3.42 (2H, tt, J = 2.1, 11.5 Hz), 3.68 (3H, s), 3.84 (1H, d, J = 13.5 Hz), |
| | 3.90-3.93 (2H, m), 4.12 (1H, d, J = 13.5 Hz), 4.56 (1H, dd, J = 4.7, 12.1 Hz), 6.71 (1H, |
| | d, J = 9.2 Hz), 7.01 (1H, dd, J = 2.1, 7.9 Hz), 7.16-7.21 (2H, m), 7.80 (1H, d, |
| | J = 3.1 Hz), 7.95 (1H, d, J = 2.1 Hz), 11.80 (1H, s). |
| 43 | (DMSO-d6) 0.98-1.01 (1H, m), 1.15-1.24 (1H, m), 1.67-1.82 (2H, m), |
| | 1.95-2.00 (4H, m), 2.05-2.09 (1H, m), 2.16 (3H, s), 2.32-2.38 (1H, m), |
| | 2.42 (3H, s), 2.61-2.78 (4H, m), 3.07-3.14 (2H, m), 3.59-3.63 (1H, m), |
| | 4.02 (1H, d, J = 13.1 Hz), 4.28 (1H, d, J = 13.2 Hz), 4.57-4.61 (1H, m), |
| | 6.73 (1H, d, J = 8.6 Hz), 7.02 (1H, dd, J = 1.4, 7.9 Hz), 7.24 (1H, d, J = 7.7 Hz), |
| | 7.36 (1H, dd, J = 2.3, 8.6 Hz), 7.87-7.94 (2H, m), 10.83 (1H, s). |
| 44 | 1.04-1.13 (1H, m), 1.23-1.46 (1H, m), 1.76-2.02 (2H, m), 2.37 (3H, s), |
| | 2.37-2.40 (1H, m), 2.60-2.77 (3H, m), 2.78-2.90 (1H, m), 3.19-3.39, (1H, |
| | m), 3.97 (1H, d, J = 13.3 Hz), 4.27 (1H, d, J = 13.3 Hz), 4.69 (1H, dd, |
| | J = 4.7 Hz, 24.0 Hz), 6.44-6.57 (2H, m), 7.02-7.18 (2H, m), 7.31-7.40 (1H, |
| | m), 8.07 (1H, m), 8.19 (1H, s), 10.40 (1H, br) |
| 45 | (MeOH, d3) d 1.03-1.08 (1H, m), 1.278-1.33 (1H, m), 1.84-1.93 (2H, m), |
| | 2.14 (3H, s), 2.37 (3H, s), 2.38-2.41 (1H, m), 2.50-2.95 (4H, m), |
| | 3.29-3.31 (1H, m), 3.88 (1H, d, J = 13.4 Hz), 4.15 (1H, d, J = 12.9 Hz), |
| | 4.65-4.69 (1H, m), 6.65 (1H, d, J = 8.7 Hz), 7.28-7.36 (3H, m), 7.82 (1H, d, J |
| | 2.2 Hz), 8.00 (1H, brs). |
| 46 | 1.10-1.14 (1H, m), 1.34 (3H, t, J = 7.7 Hz), 1.36-1.39 (1H, m), |
| | 1.80-1.98 (2H, m), 2.42 (1H, dt, J = 3.0, 12.9 Hz), 2.68-2.79 (6H, m), 3.28-3.34 (1H, |
| | m), 3.99 (1H, d, J = 13.1 Hz), 4.29 (1H, d, J = 13.1 Hz), 4.70 (1H, dd, J = 5.2, |
| | 13.1 Hz), 6.50-6.58 (2H, m), 7.09-7.18 (2H, m), 7.38 (1H, dt, J = 2.0, 7.2 Hz), |
| | 8.17 (1H, d, J = 1.5 Hz), 8.21 (1H, s), 9.70 (1H, brs). |
| 47 | 0.97 (3H, t, J = 7.4 Hz), 1.02-1.13 (1H, m), 1.33-1.44 (2H, m), 1.79 (2H, q, |
| | J = 7.4 Hz), 1.82-1.93 (1H, m), 2.43 (1H, dt, J = 2.7, 12.6 Hz), |
| | 2.68-2.74 (6H, m), 3.28-3.34 (1H, m), 3.97 (1H, d, J = 13.4 Hz), 4.29 (1H, d, = 13.1 Hz), |
| | 4.74 (1H, dd, J = 5.0, 12.9 Hz), 6.52 (1H, t, J = 2.9 Hz), 6.54 (1H, s), |
| | 7.13 (1H, s), 7.15 (1H, d, J = 2.2 Hz), 7.32 (1H, dt, J = 2.0, 7.2 Hz), |
| | 8.08 (1H, s), 8.20 (1H, brs), 9.40 (1H, brs). |
| 48 | 0.97-0.99 (4H, m), 1.09-1.14 (1H, m), 1.34 (1H, dq, J = 4.0, 12.4 Hz), |
| | 1.91-1.97 (3H, m), 2.40 (1H, dt, J = 2.7, 12.9 Hz), 2.62-2.69 (3H, m), |
| | 2.82 (1H, dt, J = 3.5, 13.1 Hz), 3.20-3.30 (1H, m), 3.97 (1H, d, J = 13.4 Hz), |
| | 4.29 (1H, d, J = 13.1 Hz), 4.69 (1H, dd, J = 4.7, 13.4 Hz), 6.51-6.54 (2H, m), |
| | 7.12 (1H, s), 7.14 (1H, d, J = 2.2 Hz), 7.38 (1H, dt, J = 2.0, 6.9 Hz), |
| | 8.08 (1H, dd, J = 2.0, 5.4 Hz), 8.18 (1H, brs), 10.00 (1H, brs). |
| 49 | 1.11-1.53 (6H, m), 1.67-1.97 (5H, m), 2.07-2.10 (2H, m), 2.43 (1H, dt, |
| | J = 2.7, 15.6 Hz), 2.68-2.95 (5H, m), 3.28-3.38 (1H, m), 3.99 (1H, d, |
| | J = 13.4 Hz), 4.30 (1H, d, J = 12.6 Hz), 4.73 (1H, dd, J = 5.2, 12.9 Hz), |
| | 6.50-6.64 (2H, m), 7.12 (1H, d, J = 8.4 Hz), 7.15 (1H, dd, J = 2.2, 8.4 Hz), |
| | 7.39 (1H, dt, J = 1.7, 8.9 Hz), 8.08 (1H, d, J = 3.7 Hz), 8.28 (1H, d, J = 2.2 Hz), |
| | 9.17 (1H, brs). |
| 50 | 1.16-1.26 (2H, m), 1.35-1.97 (12H, m), 2.45 (1H, t, J = 11.6 Hz), |
| | 2.61-2.84 (1H, m), 3.32-3.44 (4H, m), 4.00 (1H, d, J = 11.4 Hz), 4.34 (1H, m), |
| | 4.69 (1H, dd, J = 5.9, 13.1 Hz), 6.49-6.63 (3H, m), 7.05-7.13 (1H, m), 7.38 (1H, |
| | dt, J = 2.2, 8.9 Hz), 8.10 (1H, d, J = 3.3 Hz), 8.15-8.17 (1H, m). |
| 51 | 1.16-1.24 (1H, m), 1.39-1.48 (1H, m), 1.72-2.01 (4H, m), 2.30-2.42 (6H, |
| | m), 2.48-2.87 (5H, m), 3.20-3.28 (1H, m), 3.42-3.51 (2H, m), 3.99 (1H, d, |
| | J = 12.1 Hz), 4.33 (1H, d, J = 12.1 Hz), 4.69-4.71 (1H, m), 6.50-6.67 (3H, |
| | m), 7.09-7.14 (1H, m), 7.09-7.14 (1H, m), 7.30-7.44 (1H, m), 8.09 (1H, d, |
| | J = 3.0 Hz), 8.18 (1H, s), 8.66 (1H, brs). |
| 52 | (DMSO-d6) 0.96-0.99 (1H, m), 1.10-1.23 (1H, m), 1.67-1.77 (1H, m), |
| | 1.81 (1H, d, J = 11.7 Hz), 2.16 (3H, s), 2.29 (3H, s), 2.31-2.44 (1H, m), |
| | 2.52-2.57 (4H, m), 2.65-2.81 (4H, m), 3.01-3.13 (2H, m), 3.35-3.43 (4H, |
| | m), 4.00 (1H, d, J = 13.2 Hz), 4.27 (1H, d, J = 12.9 Hz), 4.61 (1H, dd, J = 4.3, |
| | 12.6 Hz), 6.72 (1H, d, J = 8.6 Hz), 7.25 (1H, dd, J = 2.4, 8.3 Hz), 7.35 (1H, |
| | dd, J = 2.0, 8.6 Hz), 7.43 (1H, d, J = 8.3 Hz), 7.94 (1H, s), 8.07 (1H, d, |
| | J = 2.4 Hz), 11.25 (1H, s) |
| 53 | 1.13-1.18 (1H, m), 1.31-1.41 (1H, m), 1.78-1.88 (2H, m), 1.96-2.00 (1H, |
| | m), 2.41 (1H, t, J = 11.9 Hz), 2.65-2.76 (3H, m), 2.88-3.09 (1H, m), |
| | 3.37-3.46 (1H, m), 3.96 (1H, d, J = 12.6 Hz), 4.32 (1H, d, J = 12.6 Hz), 4.71 (1H, |
| | d, J = 8.7 Hz), 6.51 (2H, d, J = 6.9 Hz), 7.14-7.17 (1H, m), 7.24 (1H, s), |
| | 7.34-7.42 (3H, m), 7.97 (2H, d, J = 6.9 Hz), 8.07 (1H, s), 8.41 (1H, s), |
| | 10.84 (1H, s). |
| 54 | 1.08-1.28 (1H, m), 1.29-1.51 (2H, m), 1.80-2.13 (2H, m), 2.40-2.50 (1H, |
| | m), 2.63-3.00 (3H, m), 3.35-3.54 (1H, m), 3.93-4.04 (1H, m), |
| | 4.25-4.38 (1H, m), 4.68-4.84 (1H, m), 6.43-6.57 (2H, m), 7.01-7.11 (1H, m), |
| | 7.13-7.32 (2H, m),7.33-7.49 (2H, m), 7.65-7.78 (2H, m), 8.08 (1H, d, |
| | J = 1.9 Hz), 8.05 (1H, s), 10.40 (1H, s) |
| 55 | 1.05-1.20 (1H, m), 1.30-1.48 (1H, m), 1.50-1.72 (2H, m), 1.76-1.91 (1H, |
| | m), 1.92-2.09 (1H, m), 2.31-2.55 (1H, m), 2.57-2.82 (2H, m), |
| | 2.83-3.03 (2H, m), 3.40-3.60 (1H, m), 3.87-4.03 (1H, m), 4.30-4.41 (1H, m), |
| | 4.75-4.93 (1H, m), 6.43-6.61 (2H, m), 7.05-7.46 (4H, m), 8.10 (1H, s), |
| | 8.33-8.50 (2H, m), 9.67 (1H, s) |
| 56 | 1.09-1.95 (4H, m), 2.43 (1H, t, J = 10.6 Hz), 2.62-2.94 (4H, m), |
| | 3.20-3.31 (1H, m), 3.99 (1H, d, J = 12.9 Hz), 4.14 (1H, d, J = 5.0 Hz), 4.33 (2H, t, |
| | J = 13.1 Hz), 4.67 (1H, dd, J = 5.9, 12.9 Hz), 6.50-6.70 (3H, m), |
| | 7.13-7.41 (6H, m), 8.11 (2H, dd, J = 4.7, 16.6 Hz), 8.29 (1H, s), 8.97 (1H, s). |
| 57 | (CD3OD): 1.02-1.22 (1H, m), 1.22-1.43 (1H, m), 1.72-1.98 (2H, m), |
| | 2.32-2.52 (1H, m), 2.58-2.95 (4H, m), 3.15-3.50 (5H, m), 3.68-3.90 (4H, |
| | m), 3.97 (1H, d, J = 12.9 Hz), 4.23 (1H, d, J = 12.9 Hz), 4.65 (1H, dd, |
| | J = 5.7 Hz, 12.9 Hz), 6.57 (1H, t, J = 5.2 Hz), 6.71 (1H, d, J = 5.2 Hz), |
| | 7.21 (1H, dd, J = 2.3 Hz, 8.4 Hz), 7.30 (1H, d, J = 8.4 Hz), 7.40-7.60 (1H, m), |
| | 7.85-8.10 (2H, m). |
| 58 | (DMSO-d6): 0.90 (1H, d, J = 12.3 Hz), 1.05-1.17 (1H, m), 1.58-1.70 (1H, |
| | m), 1.75 (1H, d, J = 12.3 Hz), 2.16 (3H, s), 2.37 (1H, t, J = 10.7 Hz), |
| | 2.61-2.74 (4H, m), 2.96-3.08 (1H, m), 3.22-3.38 (3H, m), 3.70 (4H, t, |
| | J = 4.3 Hz), 3.73-3.77 (1H, m), 4.00 (1H, d, J = 12.7 Hz), 4.28 (1H, d, |
| | J = 13.3 Hz), 4.50-4.57 (1H, m), 6.40 (1H, d, J = 5.0 Hz), 6.56 (1H, s), |
| | 7.20 (1H, dd, J = 2.5, 8.4 Hz), 7.38 (1H, d, J = 8.4 Hz), 7.89 (1H, d, J = 5.0 Hz), |
| | 8.01 (1H, s), 11.28 (1H, s). |
| 59 | (DMSO-d6): 0.85-1.0 (1H, m), 1.03-1.25 (1H, m), 1.56-1.83 (2H, m), |
| | 2.10 (3H, s), 2.25-2.43 (1H, m), 2.55-2.58 (3H, m), 2.92-3.13 (1H, m), |
| | 3.20-3.45 (5H, m), 3.63-3.70 (4H, m), 3.96 (1H, d, J = 13.1 Hz), 4.23 (1H, |
| | d, J = 13.1 Hz), 4.57 (1H, dd, J = 5.0 Hz, 13.1 Hz), 6.67 (1H, d, J = 8.6 Hz), |
| | 7.20 (1H, dd, J = 2.7 Hz, 8.6 Hz), 7.31 (1H, dd, J = 2.7 Hz, 8.6 Hz), 7.39 (1H, |
| | d, J = 8.6 Hz), 7.89 (1H, d, J = 2.7 Hz), 8.03 (1H, d, J = 2.7 Hz), 11.29 (1H, |
| | brs). |
| 60 | 1.01-1.30 (3H, m), 1.67-1.70 (1H, m), 1.76-1.84 (1H, m), 2.17 (3H, s), |
| | 2.19-2.24 (1H, m), 2.44-2.58 (2H, m), 2.69-2.76 (1H, m), 3.13-3.22 (5H, |
| | m), 3.67-3.70 (4H, m), 3.86 (1H, d, J = 12.2 Hz), 4.17 (1H, d, J = 13.3 Hz), |
| | 4.57-4.61 (1H, m), 6.17 (1H, d, J = 8.4 Hz), 6.24 (1H, d, J = 7.2 Hz), |
| | 7.02 (2H, d, J = 4.0 Hz), 7.13-7.20 (2H, m), 8.05 (1H, d, J = 6.7 Hz), 8.26 (1H, |
| | s). |
| 61 | 1.05-1.08 (1H, m), 1.18 (1H, s), 1.30 (3H, t, J = 7.6 Hz), 1.75-1.78 (1H, |
| | m), 1.85-1.92 (1H, m), 2.27 (3H, s), 2.30-2.34 (1H, m), 2.36 (3H, s), |
| | 2.58-2.85 (6H, m), 3.28-3.37 (1H, m), 3.93 (1H, d, J = 13.2 Hz), 4.24 (1H, |
| | d, J = 13.0 Hz), 4.67-4.72 (1H, m), 6.28 (1H, d, J = 8.5 Hz), 6.34 (1H, d, J = 7.2 Hz), |
| | 6.96-7.04 (2H, m), 7.24 (1H, t, J = 7.8 Hz), 7.91 (1H, s). |
| 62 | 0.94 (3H, t, J = 7.4 Hz), 1.04-1.07 (1H, m), 1.26-1.36 (1H, m), |
| | 1.68-1.78 (3H, m), 1.84-1.94 (1H, m), 2.26 (3H, s), 2.27-2.31 (1H, m), 2.35 (3H, s), |
| | 2.55-2.72 (5H, m), 2.79-2.89 (1H, m), 3.27-3.36 (1H, m), 3.92 (1H, d, J = 13.1 Hz), |
| | 4.25 (1H, d, J = 13.1 Hz), 4.66-4.70 (1H, m), 6.26 (1H, d, J = 8.5 Hz), |
| | 6.33 (1H, d, J = 7.3 Hz), 6.94-6.97 (1H, m), 7.01-7.03 (1H, m), |
| | 7.19-7.23 (1H, m), 7.93 (1H, s). |
| 63 | 1.01-1.12 (2H, m), 1.25-1.37 (1H, m), 1.66-1.69 (1H, m), 1.74-1.80 (1H, |
| | m), 2.17 (3H, s), 2.26 (3H, s), 2.45-2.58 (3H, m), 2.68-2.75 (1H, m), |
| | 3.12-3.25 (5H, m), 3.55-3.70 (4H, m), 3.85 (1H, d, J = 12.0 Hz), 4.17 (1H, |
| | d, J = 12.5 Hz), 4.53-4.56 (1H, m), 6.17 (1H, d, J = 8.4 Hz), 6.24 (1H, d, J = 7.3 Hz), |
| | 6.81-6.84 (1H, m), 6.87-6.96 (1H, m), 7.11-7.15 (1H, m), |
| | 7.84 (1H, s), 8.58 (1H, s). |
| 64 | (DMSO-d6) 0.93-0.96 (1H, m), 1.08-1.17 (1H, m), 1.63-1.72 (1H, m), |
| | 1.76-1.79 (1H, m), 2.34 (3H, s), 2.59-2.72 (4H, m), 2.98-3.11 (1H, m), |
| | 3.23-3.27 (4H, m), 3.29 (1H, s), 3.69-3.76 (7H, m), 3.98 (1H, d, J = 13.0 Hz), |
| | 4.24 (1H, d, J = 13.2 Hz), 4.53-4.57 (1H, m), 5.93-5.96 (1H, m), |
| | 6.20-6.22 (1H, m), 6.96-6.99 (1H, m), 7.17-7.24 (1H, m), 7.34-7.38 (1H, |
| | m), 7.86 (1H, d, J = 1.4 Hz), 11.10 (1H, s). |
| 65 | (DMSO-d6): 0.91 (1H, d, J = 12.1 Hz), 1.07-1.17 (1H, m), 1.59-1.88 (6H, |
| | m), 2.23 (3H, s), 2.27-2.38 (1H, m), 2.36 (3H, s), 2.58 (1H, t, J = 12.9 Hz), |
| | 2.65-2.79 (3H, m), 2.86-2.93 (1H, m), 3.04-3.13 (1H, m), 3.42 (2H, t, |
| | J = 11.2 Hz), 3.90-3.93 (2H, m), 3.99 (1H, d, J = 13.1 Hz), 4.27 (1H, d, |
| | J = 13.1 Hz), 4.56 (1H, dd, J = 4.7, 13.1 Hz), 6.41 (1H, d, J = 7.3 Hz), |
| | 6.48 (1H, d, J = 7.3 Hz), 7.01 (1H, d, J = 7.6 Hz), 7.20 (1H, d, J = 7.6 Hz), 7.32 (1H, |
| | t, J = 7.6 Hz), 7.96 (1H, s), 11.78 (1H, s). |
| 66 | 1.06 (1H, d, J = 12.4 Hz), 1.25-1.35 (1H, m), 1.77-1.96 (6H, m), 2.30 (1H, |
| | t, J = 12.4 Hz), 2.37 (3H, s), 2.55-2.68 (3H, m), 2.84 (1H, dt, J = 3.3, |
| | 13.3 Hz), 3.07 (1H, brs), 3.26-3.35 (1H, m), 3.47 (2H, t, J = 11.3 Hz), |
| | 3.72 (3H, s), 3.94 (1H, d, J = 13.0 Hz), 4.01 (2H, d, J = 11.0 Hz), 4.23 (1H, d, |
| | J = 13.0 Hz), 4.64 (1H, dd, J = 5.7, 13.0 Hz), 5.91 (1H, d, J = 7.8 Hz), |
| | 5.99 (1H, d, J = 7.8 Hz), 6.98 (1H, d, J = 7.7 Hz), 7.04 (1H, d, J = 7.7 Hz), 7.24 (1H, |
| | t, J = 7.7 Hz), 7.94 (1H, brs), 9.83 (1H, brs). |
| 67 | (DMSO-d6) d 0.91 (1H, d, J = 12.3 Hz), 1.04-1.15 (1H, m), 1.59-1.70 (1H, |
| | m), 1.77 (1H, d, J = 12.3 Hz), 2.23 (3H, s), 2.31-2.38 (1H, m), |
| | 2.58-2.74 (4H, m), 2.98-3.08 (1H, m), 3.25 (2H, t, J = 4.5 Hz), 3.28-3.38 (1H, m), |
| | 3.69 (4H, t, J = 4.5 Hz), 3.68-3.76 (1H, m), 4.01 (1H, d, J = 13.3 Hz), |
| | 4.28 (1H, d, J = 13.3 Hz), 4.52-4.58 (1H, m), 6.41 (1H, d, J = 7.2 Hz), 6.50 (1H, d, |
| | J = 8.5 Hz), 7.20 (1H, dd, J = 2.6, 8.5 Hz), 7.30-7.39 (2H, m), 8.01 (1H, d, |
| | J = 2.6 Hz), 11.28 (1H, s). |
| 68 | 0.99-1.12 (1H, m), 1.20-1.30 (1H, m), 1.64-1.67 (1H, m), 1.74-1.85 (1H, |
| | m), 2.20-2.27 (1H, m), 2.48-2.54 (3H, m), 2.64-2.71 (1H, m), |
| | 3.10-3.19 (1H, m), 3.23-3.25 (4H, m), 3.64 (3H, s), 3.67-3.69 (4H, m), 3.87 (1H, d, |
| | J = 13.2 Hz), 4.15 (1H, d, J = 13.2 Hz), 4.53-4.57 (1H, m), 5.84 (1H, d, J = 7.9 Hz), |
| | 5.91 (1H, d, J = 8.1 Hz), 6.95-7.01 (2H, m), 7.17 (1H, t, J = 8.0 Hz), |
| | 8.01 (1H, s), 8.63 (1H, s). |
| 69 | (DMSO-d6) 1.07-1.10 (1H, m), 1.38-1.46 (1H, m), 1.92-2.02 (2H, m), |
| | 2.24 (3H, s), 2.28-2.31 (1H, m), 2.42 (3H, s), 2.58-2.80 (4H, m), |
| | 2.93 (1H, d, J = 11.6 Hz), 3.14-3.17 (2H, m), 3.32 (4H, s), 3.75-3.77 (4H, m), |
| | 4.65 (1H, d, J = 9.7 Hz), 7.06 (1H, d, J = 6.9 Hz), 7.17 (1H, d, J = 4.8 Hz), |
| | 7.26 (1H, d, J = 6.8 Hz), 7.93 (1H, s), 8.13 (1H, d, J = 4.7 Hz), 8.15 (1H, s), |
| | 11.21 (1H, s). |
| 70 | (DMSO-d6): 0.97 (1H, d, J = 12.2 Hz), 1.18-1.29 (1H, m), 1.75-1.81 (2H, |
| | m), 2.17 (3H, s), 2.19-2.27 (1H, m), 2.34 (3H, s), 2.51-2.73 (4H, m), |
| | 2.97-3.12 (2H, m), 3.25 (2H, t, J = 4.6 Hz), 3.28-3.35 (2H, m), |
| | 3.46-3.50 (1H, m), 3.70 (4H, t, J = 4.6 Hz), 4.56 (1H, dd, J = 4.6, 12.5 Hz), 6.97 (1H, |
| | dd, J = 2.1, 8.9 Hz), 7.03 (1H, s), 7.18 (1H, d, J = 7.3 Hz), 7.75 (1H, s), |
| | 7.86 (1H, d, J = 1.7 Hz), 7.98 (1H, d, J = 2.5 Hz), 11.11 (1H, brs) |
| 71 | 1.14-1.26 (2H, m), 1.49-1.57 (3H, m), 1.81 (1H, d, J = 12.8 Hz), |
| | 2.01-2.15 (2H, m), 2.35 (3H, s), 2.38 (3H, s), 2.52-2.63 (1H, m), 2.74-2.86 (1H, m), |
| | 3.06 (1H, d, J = 11.7 Hz), 3.26-3.34 (5H, m), 3.78 (4H, t, J = 4.7 Hz), |
| | 4.66-4.71 (1H, m), 6.92-7.06 (4H, m), 7.93 (1H, s), 8.07 (1H, dd, J = 1.3, |
| | 4.6 Hz), 8.58 (1H, s). |
| 72 | 1.08-1.20 (1H, m), 1.80-1.90 (1H, m), 2.01-2.16 (1H, m), 2.20-2.33 (1H, |
| | m), 2.45 (3H, s), 2.48-2.63 (2H, m), 2.67-2.93 (2H, m), 3.29-3.41 (2H, |
| | m), 3.60-3.74 (2H, m), 4.77, (1H, dd, J = 4.7, 13.1 Hz), 6.70-6.85 (3H, m), |
| | 7.08-7.20 (4H, m), 8.25 (1H, s), 8.99 (1H, s) |
| 73 | (DMSO-d6) 0.80-1.02 (1H, m), 1.10-1.35 (3H, m), 1.24 (3H, t, J = 7.7 Hz), |
| | 1.67-1.92 (2H, m), 2.23 (1H, t, J = 12.1 Hz), 2.40-2.90 (2H, m), 2.65 (2H, |
| | q, J = 7.7 Hz), 2.95-3.20 (1H, m), 3.20-3.54 (1H, m), 3.68 (1H, d, |
| | J = 12.1 Hz), 4.57 (1H, dd, J = 4.7 Hz, 12.9 Hz), 6.70 (1H, t, J = 7.2 Hz), |
| | 6.82 (2H, d, J = 7.2 Hz), 7.13 (2H, t, J = 7.2 Hz), 7.25 (1H, dd, J = 1.7 Hz, 8.4 Hz), |
| | 7.43 (1H, d, J = 8.4 Hz), 8.08 (1H, dd, J = 1.7 Hz), 12.10 (1H, brs) |
| 74 | (DMSO-d6) 0.80-1.00 (1H, m), 0.92 (3H, t, J = 7.4 Hz), 1.10-1.35 (1H, m), |
| | 1.60-1.90 (2H, m), 1.69 (2H, s, J = 7.4 Hz), 2.23 (1H, t, J = 12.1 Hz), |
| | 2.40-2.65 (2H, m), 2.59 (2H, t, J = 7.4 Hz), 2.65-2.90 (2H, m), 2.87-3.18 (1H, |
| | m), 3.24-3.50 (1H, m), 3.69 (1H, d, J = 12.1 Hz), 4.58 (1H, dd, J = 4.0 Hz, |
| | 15.1 Hz), 6.70 (1H, t, J = 7.2 Hz), 6.82 (2H, d, J = 8.2 Hz), 7.13 (2H, t, |
| | J = 7.2 Hz), 7.24 (1H, dd, J = 2.5 Hz, 8.4 Hz), 7.41 (1H, d, J = 8.4 Hz), |
| | 8.09 (1H, d, J = 2.5 Hz), 11.94 (1H, s). |
| 75 | (DMSO-d6) 0.74-1.03 (5H, m), 1.10-1.32 (1H, m), 1.675-1.87 (2H, m), |
| | 1.87-2.00 (1H, m), 2.12-2.32 (1H, m), 2.40-2.65 (2H, m), 2.65-2.88 (2H, |
| | m), 2.88-3.20 (1H, m), 3.43 (1H, d, J = 12.4 Hz), 3.68 (1H, d, J = 12.4 Hz), |
| | 4.57 (1H, d, J = 9.7 Hz), 6.69 (1H, t, J = 7.4 Hz), 6.82 (2H, d, J = 7.4 Hz), |
| | 7.04-7.20 (2H, m), 7.23 (1H, dd, J = 2.2 Hz, 8.2 Hz), 7.41 (1H, d, J = 8.4 Hz), |
| | 8.05 (1H, brs), 11.92 (1H, brs). |
| 76 | (DMSO-d6) 0.84-1.05 (1H, m), 1.10-1.62 (6H, m), 1.62-2.10 (8H, m), |
| | 2.23 (1H, t, J = 11.9 Hz), 2.40-2.90 (4H, m), 2.95-3.20 (1H, m), |
| | 3.24-3.55 (1H, m), 3.68 (1H, d, J = 11.9 Hz), 4.44-4.70 (1H, m), 6.70 (1H, t, |
| | J = 6.9 Hz), 6.82 (2H, d, J = 7.7 Hz), 7.05-7.33 (1H, m), 7.12 (2H, t, |
| | J = 6.9 Hz), 7.34-7.50 (1H, m), 8.06-8.20 (1H, m), 11.87 (1H, s). |
| 77 | (DMSO-d6) 0.90-1.05 (1H, m), 1.10-1.36 (2H, m), 1.40-1.70 (6H, m), |
| | 1.70-1.90 (2H, m), 2.23 (1H, t, J = 12.4 Hz), 2.40-2.80 (4H, m), |
| | 2.90-3.27 (1H, m), 3.20-3.55 (4H, m), 3.69 (1H, d, J = 12.4 Hz), 4.55 (1H, dd, |
| | J = 5.0 Hz, 12.1 Hz), 6.69 (1H, t, J = 7.2 Hz), 6.83 (2H, d, J = 7.2 Hz), |
| | 7.14 (2H, t, J = 7.2 Hz), 7.20 (1H, dd, J = 2.5 Hz, 8.4 Hz), 7.39 (1H, d, J = 8.4 Hz), |
| | 7.90-8.05 (1H, m), 11.20 (1H, brs). |
| 78 | (DMSO-d6) 0.90-1/05 (1H, m), 1.13-1.35 (1H, m), 1.70-1.88 (2H, m), |
| | 2.23 (3H, s), 2.33-2.80 (8H, m), 2.90-3.15 (1H, m), 3.15-3.55 (6H, m), |
| | 3.69 (1H, d, J = 12.4 Hz), 4.44-4.65 (1H, m), 6.70 (1H, t, J = 7.2 Hz), |
| | 6.82 (2H, d, J = 7.2 Hz), 7.13 (2H, t, J = 7.2 Hz), 7.19 (1H, dd, J = 2.5 Hz, 8.4 Hz), |
| | 7.38 (1H, d, J = 8.4 Hz), 7.97-8.07 (1H, m), 11.23 (1H, s) |
| 79 | (DMSO-d6) 0.85-1.05 (1H, m), 1.10-1.38 (1H, m), 1.50-1.95 (3H, m), |
| | 2.22 (1H, t, J = 12.6 Hz), 2.40-3.10 (4H, m), 3.20-3.50 (1H, m), |
| | 3.55-3.85 (1H, m), 4.50-4.75 (1H, m), 6.62-6.87 (1H, m), 6.80 (2H, d, J = 8.2 Hz), |
| | 6.87-7.02 (1H, m), 7.04-7.26 (1H, m), 7.11 (2H, t, J = 8.2 Hz), |
| | 7.27-7.60 (2H, m), 7.49 (2H, d, J = 8.2 Hz), 8.01 (2H, d, J = 8.2 Hz), 8.26 (1H, s). |
| 80 | 1.10-1.20 (1H, m), 1.30-1.48 (1H, m), 1.80-1.91 (1H, m), 2.00-2.19 (1H, |
| | m), 2.20-2.32 (1H, m), 2.50-2.64 (2H, m), 2.87 (1H, s), 2.90-2.96 (2H, |
| | m), 3.28-3.40 (1H, m), 3.41-3.60 (1H, m), 3.61-3.70 (1H, m), |
| | 4.80-4.92 (1H, m), 6.72-6.88 (3H, m), 7.08-7.38 (6H, m), 8.35-8.44 (2H, m), |
| | 9.87 (1H, d, J = 6.9 Hz) |
| 81 | 1.09-1.28 (1H, m), 1.39-1.60 (2H, m), 1.77-1.93 (1H, m), 1.95-2.17 (1H, |
| | m), 2.18-2.36 (1H, m), 2.49-2.69 (2H, m), 2.80-3.04 (2H, m), |
| | 3.30-3.54 (2H, m), 3.59-3.74 (1H, m), 4.70-4.84 (1H, m), 6.70-6.90 (3H, m), |
| | 7.00-7.10 (1H, m), 7.11-7.21 (4H, m), 7.33-7.48 (1H, m), 7.60-7.78 (2H, m), |
| | 8.36 (1H, s) |
| 82 | (CD3OD) 1.00-1.20 (1H, m), 1.20-1.54 (1H, m), 1.72-2.10 (3H, m), |
| | 2.12-2.32 (1H, m), 2.42-3.08 (3H, m), 3.22-3.50 (1H, m), 3.55-3.85 (2H, m), |
| | 4.06 (2H, s), 4.67 (1H, dd, J = 5.7 Hz, 12.4 Hz), 6.71-6.92 (3H, m), |
| | 6.92-7.06 (1H, m), 7.09-7.50 (9H, m), 8.09 (1H, brs). |
| 83 | (DMSO-d6) 0.90-1.05 (1H, m), 1.18-1.42 (1H, m), 1.60-1.95 (2H, m), |
| | 2.14-2.35 (2H, m), 2.29 (3H, s), 2.40-2.85 (4H, m), 2.90-3.20 (2H, m), |
| | 4.60 (1H, dd, J = 4.0 Hz, 13.4 Hz), 6.75-7.17 (5H, m), 7.25 (1H, dd, |
| | J = 2.5 Hz, 8.4 Hz), 7.42 (1H, d, J = 8.4 Hz), 8.04 (1H, d, J = 2.5 Hz). |
| 84 | (DMSO-d6) 0.84-0.95 (1H, m), 1.08-1.30 (2H, m), 1.64-1.85 (2H, m), |
| | 2.18-2.36 (1H, m), 2.29 (3H, s), 2.45-2.85 (3H, m), 2.95-3.18 (1H, m), |
| | 3.48 (1H, d, J = 12.6 Hz), 3.74 (1H, d, J = 12.6 Hz), 4.58 (1H, dd, J = 4.2 Hz, |
| | 12.6 Hz), 6.38-6.52 (1H, m), 6.54-6.68 (2H, m), 7.05-7.20 (1H, m), |
| | 7.25 (1H, dd, J = 2.5 Hz, 8.4 Hz), 7.43 (1H, d, J = 8.4 Hz), 8.05 (1H, m). |
| 85 | (DMSO-d6) 1.30-1.54 (1H, m), 1.63-1.88 (2H, m), 2.05-2.48 (2H, m), |
| | 2.60-2.90 (1H, m), 2.82 (3H, s), 2.90-3.17 (2H, m), 3.17-3.40 (2H, m), |
| | 3.50-3.72 (1H, m), 3.95-4.25 (1H, m), 5.00-5.20 (1H, m), 7.20-7.42 (2H, |
| | m), 7.42-7.60 (2H, m), 7.62-7.88 (1H, m), 7.88-8.05 (1H, m), 8.56 (1H, |
| | m). |
| 86 | (DMSO-d6) 0.90-1.05 (1H, m), 1.20-1.45 (1H, m), 1.80-2.00 (2H, m), |
| | 2.14-2.35 (1H, m), 2.28 (3H, s), 2.40-2.62 (2H, m), 2.65-2.90 (2H, m), |
| | 2.90-3.40 (3H, m), 4.52-4.70 (1H, m), 6.90-7.09 (2H, m), 7.12-7.30 (2H, |
| | m), 7.30-7.38 (1H, m), 7.43 (1H, d, J = 8.4 Hz), 8.05 (1H, s), 12.0 (1H, |
| | brs). |
| 87 | 1.10-1.54 (1H, m), 1.42-4.17 (1H, m), 1.81-1.86 (1H, m), 2.00-2.06 (1H, |
| | m), 2.31-2.38 (1H, m), 2.42 (3H, m), 2.45-2.62 (2H, m), 2.71-2.86 (2H, |
| | m), 3.33-3.42 (2H, m), 3.67 (1H, d, J = 13.1 Hz), 4.77 (1H, dd, J = 4.9, |
| | 13.7 Hz), 6.73-6.74 (3H, m), 7.13-7.25 (3H, m), 8.21 (1H, s), 9.00 (1H, |
| | brs). |
| 88 | (DMSO-d6) 0.80-0.98 (1H, m), 1.08-1.30 (1H, m), 1.65-1.90 (2H, m), |
| | 2.15-2.36 (1H, m), 2.28 (3H, s), 2./42-2.67 (2H, m), 2.67-2.90 (2H, m), |
| | 2.95-3.18 (1H, m), 3.22-3.50 (1H, m), 3.68 (1H, d, J = 12.6 Hz), |
| | 4.50-4.65 (1H, m), 6.83 (2H, d, J = 8.7 Hz), 7.14 (2H, d, J = 8.7 Hz), 7.19-7.30 (1H, m), |
| | 7.42 (1H, d, J = 8.2 Hz), 8.07 (1H, s), 11.96 (1H, brs). |
| 89 | (DMSO-d6) 1.02-1.15 (1H, m), 1.40-1.60 (1H, m), 1.80-1.95 (1H, m), |
| | 1.95-2.30 (2H, m), 2.20 (3H, s), 2.38 (3H, s), 2.30-2.53 (1H, m), |
| | 2.53-2.67 (1H, m), 2.72-3.00 (3H, m), 3.00-3.13 (1H, m), 3.25-3.42 (1H, m), |
| | 4.65-4.77 (1H, m), 6.80-6.93 (2H, m), 6.97-7.11 (2H, m), 7.17-7.41 (2H, |
| | m), 8.01 (1H, s). |
| 90 | (DMSO-d6) d 0.89-0.93 (1H, m), 1.19-2.23 (1H, m), 1.75-1.80 (2H, m), |
| | 2.19 (3H, s), 2.28 (3H, s), 2.44-2.59 (2H, m), 2.74-2.80 (1H, m), |
| | 2.93 (1H, m), 3.03-3.08 (1H, m), 3.35-3.43 (1H, m), 3.67 (2H, d, J = 12.4 Hz), |
| | 4.56 (1H, dd, J = 4.5, 12.6 Hz), 6.52 (1H, d, J = 7.2 Hz), 6.59-6.64 (1H, m), |
| | 7.01 (1H, t, J = 7.9 Hz), 7.22-7.27 (2H, m), 7.43 (1H, d, J = 8.4 Hz), |
| | 8.06 (1H, s), 11.98 (1H, s). |
| 91 | (DMSO-d6) 0.85-0.98 (1H, m), 1.13-1.33 (1H, m), 1.68-1.88 (2H, m), |
| | 2.05-2.23 (1H, m), 2.15 (3H, s), 2.28 (3H, s), 2.36-2.64 (2H, m), |
| | 2.65-2.85 (1H, m), 2.98-3.18 (1H, m), 3.22-3.42 (1H, m), 3.59 (1H, d, |
| | J = 12.4 Hz), 4.58 (1H, dd, J = 4.2, 13.1 Hz), 6.71 (2H, d, J = 8.4 Hz), |
| | 6.94 (2H, d, J = 8.4 Hz), 7.23 (1H, dd, J = 2.7 Hz, 8.4 Hz), 7.40 (1H, d, J = 8.4 Hz), |
| | 8.08 (1H, d, J = 2.7 Hz), 11.96 (1H, s). |
| 92 | (DMSO-d6) 0.87-1.00 (1H, m), 1.22-1.42 (1H, m), 1.74-1.96 (2H, m), |
| | 1.98-2.15 (1H, m), 2.22-2.40 (1H, m), 2.29 (3H, m), 2.65-2.87 (2H, m), |
| | 2.88-2.98 (1H, m), 2.98-3.20 (2H, m), 3.60-3.84 (1H, m), 3.72 (3H, s), |
| | 4.54-4.68 (1H, m), 6.70-7.00 (4H, m), 7.12-7.34 (2H, m), 7.41 (1H, d, |
| | J = 8.4 Hz), 7.92-8.20 (1H, m), 11.97 (1H, brs). |
| 93 | (DMSO-d6) 0.98-1.15 (1H, m), 1.22-1.52 (2H, m), 1.73-2.06 (2H, m), |
| | 2.10-2.30 (1H, m), 2.30-2.70 (2H, m), 2.40 (3H, s), 2.75-3.00 (2H, m), |
| | 3.12-3.45 (2H, m), 3.55-3.80 (1H, m), 3.69 (3H, s), 4.60-4.78 (1H, m), |
| | 6.25-6.52 (2H, m), 6/43 (1H, d, J = 8.9 Hz), 7.04 (1H, t, J = 8.9 Hz), |
| | 7.20-7.40 (1H, m), 7.27 (1H, dd, J = 2.2, 8.4 Hz), 7.99 (1H, d, J = 2.2 Hz). |
| 94 | (CD3OD) 1.04-1.15 (1H, m), 1.35-1.55 (1H, m), 1.82-2.06 (2H, m), |
| | 2.06-2.22 (1H, m), 2.30-2.50 (1H, m), 2.38 (3H, s), 2.50-2.65 (1H, m), |
| | 2.75-3.00 (2H, m), 3.17 (1H, d, J = 12.1 Hz), 3.24-3.40 (1H, m), 3.45 (1H, d, |
| | J = 12.1 Hz), 3.58 (3H, s), 4.64-4.76 (1H, m), 6.74 (2H, d, J = 9.2 Hz), |
| | 6.83 (2H, d, J = 9.2 Hz), 7.25 (1H, dd, J = 2.5 Hz, 8.4 Hz), 7.33 (1H, d, J = 8.4 Hz), |
| | 8.01 (1H, brs). |
| 95 | 1.15 (3H, t, J = 7.4 Hz), 1.16-1.18 (1H, m), 1.40-1.58 (1H, m), |
| | 1.80-1.91 (1H, m), 2.05-2.31 (2H, m), 2.46 (3H, s), 2.47-2.49 (1H, m), 2.52 (2H, q, |
| | J = 7.4 Hz), 2.70-2.94 (2H, m), 3.26-3.46 (2H, m), 3.56-3.68 (1H, m), |
| | 4.73-4.82 (1H, m), 6.75 (2H, d, J = 8.4 Hz), 7.02 (2H, d, J = 8.7 Hz), |
| | 7.10-7.23 (3H, m), 8.21 (1H, s), 9.00 (1H, brs) |
| 96 | 0.88 (3H, t, J = 7.4 Hz), 0.98-1.21 (2H, m), 1.41-1.63 (3H, m), |
| | 1.78-2.32 (4H, m), 2.44 (3H, s), 2.47-2.60 (1H, m), 2.61-2.92 (2H, m), |
| | 3.23-3.42 (2H, m), 3.56-3.70 (1H, m), 4.70-4.80 (1H, m), 5.30-5.58 (1H, m), |
| | 6.73 (1H, d, J = 8.6 Hz), 6.80-6.89 (1H, m), 6.92-7.00 (1H, m), 7.03-7.20 (3H, |
| | m), 8.24 (1H, s), 9.57 (1H, s) |
| 97 | 1.15-1.22 (6H, m), 1.76-2.10 (3H, m), 2.18-2.34 (1H, m), 2.44 (3H, s), |
| | 2.45-2.60 (1H, m), 2.61-2.91 (3H, m), 3.25-3.40 (1H, m), 3.53-3.71 (2H, |
| | m), 4.69-5.80 (1H, m), 5.50-5.73 (1H, m), 6.75 (2H, d, J = 8.7 Hz), |
| | 6.86 (2H, d, J = 8.7 Hz), 6.94-7.18 (4H, m), 8.19 (1H, s) |
| 98 | 1.37-1.59 (2H, m), 1.72-1.88, (2H, m), 2.40-2.57 (2H, m), 2.63-2.73 (1H, |
| | m), 3.72 (3H, s), 3.81 (1H, d, J = 14.6 Hz), 4.53-4.73 (2H, m), 5.62 (1H, d, |
| | J = 14.6 Hz), 5.98 (1H, s), 6.39 (1H, dd, J = 4.6, 4.6 Hz), 7.00-7.11 (2H, m), |
| | 7.16-7.21 (2H, m), 7.26-7.32 (1H, m), 8.22 (2H, d, J = 4.6 Hz) |
| 99 | 1.24-1.59 (4H, m), 1.71-2.10 (10H, m), 2.43-2.62 (2H, m), 2.69-2.80 (1H, |
| | m), 3.72-3.78 (1H, m), 3.82 (1H, d, J = 14.6 Hz), 4.54-4.76 (2H, m), |
| | 5.64 (1H, d, J = 14.6 Hz), 5.84 (1H, s), 6.66 (1H, d, J = 4.9 Hz), 7.00-7.09 (2H, |
| | m), 7.18-7.22 (2H, m), 7.27-7.33 (1H, m), 8.40 (1H, d, J = 4.9 Hz) |
| 100 | 1.41-1.61 (1H, m), 1.68-1.93 (3H, m), 2.39-2.49 (1H, m), 2.50-2.60 (1H, |
| | m), 2.64-2.74 (1H, m), 3.70 (3H, s), 3.79 (1H, d, J = 14.6 Hz), |
| | 4.55-4.74 (2H, m), 5.58 (1H, d, J = 14.6 Hz), 6.12 (1H, s), 6.41 (1H, dd, J = 4.7, |
| | 4.7 Hz), 6.71-6.81 (2H, m), 7.13-7.18 (2H, m), 8.23 (2H, d, J = 4.7 Hz) |
| 101 | 1.41 (3H, t, J = 7.3 Hz), 1.42-1.58 (2H, m), 2.41-2.52 (1H, m), |
| | 2.56-2.66 (1H, m), 2.71-2.81 (1H, m), 3.80 (1H, d, J = 14.3 Hz), 4.02 (2H, q, |
| | J = 7.3 Hz), 4.57-4.77 (2H, m), 5.60 (1H, d, J = 14.3 Hz), 5.94 (1H, s), |
| | 6.67 (1H, d, J = 4.7 Hz), 6.70-6.75 (1H, m), 6.77-6.81 (1H, m), 7.14-7.19 (1H, |
| | m), 7.20 (1H, s), 8.41 (1H, d, J = 4.7 Hz) |
| 102 | 1.38-1.56 (2H, m), 1.71-1.88 (2H, m), 2.35 (3H, s), 2.44-2.76 (3H, m), |
| | 3.70 (3H, s), 3.78 (1H, d, J = 14.6 Hz), 4.53-4.73 (2H, m), 5.60 (1H, d, |
| | J = 14.6 Hz), 5.84 (1H, s), 6.39 (1H, dd, J = 4.9, 4.9 Hz), 6.82 (1H, d, |
| | J = 1.3 Hz), 6.86 (1H, dd, J = 1.3, 7.9 Hz), 7.06 (1H, d, J = 7.9 Hz), 7.16 (1H, |
| | s), 8.22 (2H, d, J = 4.9 Hz) |
| 103 | 1.42-1.92 (4H, m), 2.41-2.77 (3H, m), 3.70 (3H, s), 3.78 (1H, d, |
| | J = 14.6 Hz), 4.56-4.74 (2H, m), 5.58 (1H, d, J = 14.6 Hz), 6.09 (1H, s), |
| | 6.40 (1H, dd, J = 4.9, 4.9 Hz), 7.01-7.05 (1H, m), 7.10-7.18 (2H, m), |
| | 7.25-7.26 (1H, m), 8.24 (2H, d, J = 4.9 Hz) |
| 104 | 1.30-2.10 (4H, m), 2.35-3.10 (3H, m), 3.83 (1H, d, J = 14.8 Hz), 4.58 (1H, |
| | d, J = 13.6 Hz), 4.71 (1H, d, J = 13.6 Hz), 5.64 (1H, d, J = 14.8 Hz), 6.32 (1H, |
| | s), 6.38 (1H, t, J = 9.4 Hz), 6.87 (1H, s), 6.99 (1H, d, J-8.4 Hz), 7.10 (1H, d, |
| | J = 8.4 Hz), 7.25-7.60 (6H, m), 8.21 (2H, d, J = 4.7 Hz) |
| 105 | 1.15 (3H, t, J = 7.7 Hz), 1.40-1.91 (4H, m), 2.42 (2H, q, J = 7.7 Hz), |
| | 2.40-2.49 (1H, m), 2.52-2.62 (1H, m), 2.65-2.76 (1H, m), 3.86 (1H, d, |
| | J = 14.6 Hz), 4.53-4.71 (2H, m), 5.70 (1H, d, J = 14.6 Hz), 6.14 (1H, s), |
| | 6.88 (1H, d,, J = 2.2 Hz), 7.03 (1H, dd, J = 2.2, 8.4 Hz), 7.15 (1H, d, J = 8.4 Hz), |
| | 7.39 (1H, s), 7.43-7.47 (3H, m), 7.53-7.61 (2H, m), 8.08-8.15 (2H, m) |
| 106 | 1.24-2.03 (4H, m), 2.42-2.53 (2H, m), 2.62-2.71 (1H, m), 3.72 (3H, s), |
| | 3.82 (1H, d, J = 14.3 Hz), 4.09-4.23 (2H, m), 5.62 (1H, d, J = 14.3 Hz), |
| | 6.04 (1H, s), 6.50-6.58 (2H, m), 7.00-7.08 (2H, m), 7.18-7.31 (3H, m), |
| | 7.36-7.42 (1H, m), 8.10 (1H, d, J = 4.2 Hz) |
| 107 | (DMSO-d6) 1.23 (3H, t, J = 7.1 Hz), 1.20-1.27 (2H, m), 1.53-1.69 (2H, m), |
| | 2.09 (3H, s), 2.3-2.41 (2H, m), 2.54 (1H, dt, J = 2.1, 12.5 Hz), 3.73 (1H, d, |
| | J = 14.3 Hz), 4.01-4.13 (3H, m), 4.16 (1H, d, J = 13.4 Hz), 5.34 (1H, d, |
| | J = 14.3 Hz), 6.66 (1H, d, J = 8.8 Hz), 6.81-6.87 (1H, m), 7.10 (1H, s), |
| | 7.15 (1H, dd, J = 3.3, 10.6 Hz), 7.28-7.33 (2H, m), 7.87 (1H, d, J = 2.3 Hz), |
| | 8.53 (1H, s). |
| 108 | (DMSO-d6) 1.21-1.28 (2H, m), 1.59 (9H, s), 1.60-1.71 (2H, m), |
| | 2.09 (3H, s), 2.34-2.42 (2H, m), 2.50-2.58 (1H, m), 3.74 (1H, d, J = 14.6 Hz), |
| | 4.03 (1H, d, J = 13.1 Hz), 4.15 (1H, d, J = 13.1 Hz), 5.34 (1H, d, J = 14.6 Hz), |
| | 6.66 (1H, d, J = 8.6 Hz), 6.89 (1H, dt, J = 2.8, 8.4 Hz), 7.04 (1H, s), |
| | 7.28-7.38 (3H, m), 7.54 (1H, s), 7.87 (1H, d, J = 2.1 Hz) |
| 109 | 1.24-2.03 (4H, m), 2.34 (3H, s), 2.42-2.57 (2H, m), 2.64-2.72 (1H, m), |
| | 3.71 (3H, s), 3.78 (1H, d, J = 14.3 Hz), 4.09-4.24 (2H, m), 5.60 (1H, d, |
| | J = 14.3 Hz), 5.88 (1H, s), 6.50-6.59 (2H, m), 6.81-6.87 (2H, m), 7.06 (1H, |
| | d, J = 7.7 Hz), 7.17 (1H, s), 7.37-7.42 (1H, m), 8.10 (1H, d, J = 4.2 Hz) |
| 110 | (DMSO-d6) 1.20-1.34 (1H, m), 1.43-1.54 (1H, m), 1.68-1.77 (2H, m), |
| | 2.15 (3H, s), 2.20-2.31 (2H, m), 2.31 (3H, s), 2.39-2.48 (1H, m), |
| | 3.20 (1H, d, J = 12.9 Hz), 3.31-3.38 (1H, m), 3.68 (3H, s), 3.70 (1H, d, |
| | J = 14.4 Hz), 5.39 (1H, d, J = 14.4 Hz), 6.83-6.88 (2H, m), 7.08 (1H, s), |
| | 7.14 (2H, d, J = 7.6 Hz), 7.44 (1H, d, J = 7.1 Hz), 8.00 (1H, dd, J = 1.4, 4.9 Hz), |
| | 8.31 (1H, s) |
| 111 | (DMSO-d6): 1.21-1.27 (2H, m), 1.51-1.63 (1H, m), 1.63-1.70 (1H, m), |
| | 2.16 (3H, s), 2.31 (3H, s), 2.34-2.47 (2H, m), 2.50-2.58 (1H, m), |
| | 3.68 (3H, s), 3.69 (1H, d, J = 14.4 Hz), 4.09 (1H, d, J = 13.1 Hz), 4.23 (1H, d, |
| | J = 13.1 Hz), 5.35 (1H, d, J = 14.4 Hz), 6.40 (1H, d, J = 5.0 Hz), 6.56 (1H, s), |
| | 6.83 (1H, dd, J = 1.4, 8.0 Hz), 7.05 (1H, s), 7.10 (1H, s), 7.14 (1H, d, |
| | J = 1.4 Hz), 7.89 (1H, d, J = 5.0 Hz), 8.30 (1H, s) |
| 112 | (DMSO-d6) 1.18-1.31 (2H, m), 1.52-1.60 (1H, m), 1.62-1.70 (1H, m), |
| | 2.09 (3H, s), 2.30 (3H, s), 2.36-2.42 (2H, m), 2.50-2.58 (1H, m), 3.68 (3H, |
| | s), 3.72 (1H, d, J = 14.5 Hz), 4.00-4.05 (1H, m), 4.16 (1H, d, J = 13.2 Hz), |
| | 5.35 (1H, d, J = 14.5 Hz), 6.66 (1H, d, J = 8.6 Hz), 6.83 (1H, dd, J = 1.3, |
| | 8.0 Hz), 7.05 (1H, s), 7.14 (2H, d, J = 8.0 Hz), 7.3 (1H, dd, J = 2.4, 8.6 Hz), |
| | 7.86 (1H, d, J = 2.3 Hz), 8.31 (1H, s) |
| 113 | (DMSO-d6) 1.21-1.33 (2H, m), 1.52-1.64 (1H, m), 1.68-1.73 (1H, m), |
| | 2.31 (3H, s), 2.32-2.47 (2H, m), 2.52-2.60 (1H, m), 3.67 (3H, s), |
| | 3.70 (3H, s), 4.07 (1H, d, J = 13.3 Hz), 4.20 (1H, d, J = 13.3 Hz), 5.35 (1H, d, |
| | J = 14.5 Hz), 5.95 (1H, d, J = 7.8 Hz), 6.22 (1H, d, J = 8.1 Hz), 6.83 (1H, dd, |
| | J = 1.4, 8.0 Hz), 7.06 (1H, s), 7.13-7.18 (2H, m), 7.37 (1H, t, J = 8.0 Hz), |
| | 8.31 (1H, s) |
| 114 | 1.27-1.92 (4H, m), 2.20 (3H, s), 2.24 (3H, s), 2.32-2.47 (2H, m), |
| | 2.58-2.67 (1H, m), 3.78 (1H, d, J = 14.6 Hz), 4.05 (1H, d, J = 13.0 Hz), 4.18 (1H, |
| | d, J = 13.0 Hz), 5.19 (1H, d, J = 16.6 Hz), 5.37 (1H, d, J = 16.6 Hz), 5.60 (1H, |
| | s), 5.61 (1H, d, J = 14.6 Hz), 6.26 (1H, s), 6.38-6.40 (2H, m), 6.78 (1H, d, |
| | J = 7.8 Hz), 7.00 (1H, d, J = 7.8 Hz), 7.14-7.17 (2H, m), 7.32-7.38 (4H, m), |
| | 7.97 (1H, d, J = 5.7 Hz) |
| 115 | 1.38-1.99 (4H, m), 2.37-2.73 (3H, m), 3.70 (3H, s), 3.79 (1H, d, |
| | J = 14.6 Hz), 4.06-4.25 (2H, m), 5.59 (1H, d, J = 14.6 Hz), 6.11 (1H, s), |
| | 6.51-6.58 (2H, m), 7.02 (2H, d, J = 8.0 Hz), 7.12 (1H, d, J = 8.0 Hz), 7.17 (1H, s), |
| | 7.39 (1H, t, J = 7.2 Hz), 8.10 (1H, d, J = 3.7 Hz) |
| 116 | (DMSO-d6) 1.23-1.32 (1H, m), 1.42-1.55 (1H, m), 1.74-1.83 (2H, m), |
| | 2.15 (3H, s), 2.27-2.32 (2H, m), 2.42-2.48 (1H, m), 3.20 (1H, d, |
| | J = 12.8 Hz), 3.36 (1H, d, J = 12.8 Hz), 3.68 (3H, s), 3.74 (1H, d, J = 14.5 Hz), |
| | 5.38 (1H, d, J = 14.5 Hz), 6.85 (1H, dd, J = 4.9, 7.3 Hz), 7.05 (1H, dd, J = 2.4, |
| | 8.4 Hz), 7.11 (1H, s), 7.32 (1H, d, J = 8.4 Hz), 7.43 (2H, d, J = 2.4 Hz), |
| | 8.01 (1H, dd, J = 1.4, 4.8 Hz), 8.57 (1H, s) |
| 117 | (DMSO-d6): 1.21-1.27 (2H, m), 1.51-1.63 (1H, m), 1.66-1.71 (1H, m), |
| | 2.16 (3H, s), 2.37-2.46 (2H, m), 2.54-2.62 (1H, m), 3.68 (3H, s), |
| | 3.72 (1H, d, J = 14.5 Hz), 4.09 (1H, d, J = 13.1 Hz), 4.23 (1H, d, J = 13.1 Hz), |
| | 5.34 (1H, d, J = 14.5 Hz), 6.40 (1H, d, J = 5.0 Hz), 6.56 (1H, s), 7.04 (1H, dd, |
| | J = 2.4, 8.5 Hz), 7.08 (1H, s), 7.32 (1H, d, J = 8.5 Hz), 7.44 (1H, d, J = 2.4 Hz), |
| | 7.89 (1H, d, J = 5.0 Hz), 8.58 (1H, s) |
| 118 | (DMSO-d6) 1.18-1.31 (2H, m), 1.52-1.60 (1H, m), 1.62-1.70 (1H, m), |
| | 2.09 (3H, s), 2.36-2.42 (2H, m), 2.50-2.58 (1H, m), 3.68 (3H, s), |
| | 3.72 (1H, d, J = 14.5 Hz), 4.00-4.05 (1H, m), 4.16 (1H, d, J = 13.2 Hz), 5.33 (1H, |
| | d, J = 14.5 Hz), 6.66 (1H, d, J = 8.7 Hz), 7.04 (1H, dd, J = 2.3, 8.4 Hz), |
| | 7.09 (1H, s), 7.28 (1H, dd, J = 2.3, 8.8 Hz), 7.32 (1H, d, J = 8.4 Hz), 7.43 (1H, d, |
| | J = 2.4 Hz), 7.87 (1H, d, J = 2.2 Hz), 8.58 (1H, s) |
| 119 | 1.23-1.29 (2H, m), 1.52-1.64 (1H, m), 1.68-1.74 (1H, m), 2.37-2.48 (2H, |
| | m), 2.52-2.63 (2H, m), 3.33 (3H, s), 3.70 (3H, s), 3.72 (1H, d, J = 14.5 Hz), |
| | 4.08 (1H, d, J = 13.2 Hz), 4.20 (1H, d, J = 13.2 Hz), 5.34 (1H, d, J = 14.5 Hz), |
| | 5.95 (1H, d, J = 7.8 Hz), 6.22 (1H, d, J = 8.1 Hz), 7.04 (1H, dd, J = 2.4, |
| | 8.4 Hz), 7.09 (1H, s), 7.34 (1H, t, J = 8.4 Hz), 7.44 (1H, d, J = 2.4 Hz), |
| | 8.58 (1H, s) |
| 120 | (DMSO-d6) 1.23 (3H, t, J = 7.1 Hz), 1.26-1.30 (1H, m), 1.40-1.50 (1H, m), |
| | 1.76-1.84 (2H, m), 2.15 (3H, s), 2.27-2.33 (2H, m), 2.43-2.47 (1H, m), |
| | 3.20 (1H, d, J = 12.4 Hz), 3.75 (1H, d, J = 14.5 Hz), 3.99-4.14 (2H, m), 5.38 (1H, |
| | d, J = 14.6 Hz), 6.84-6.87 (1H, m), 7.05 (1H, dd, J = 2.3, 8.4 Hz), 7.13 (1H, |
| | s), 7.32 (1H, d, J = 8.4 Hz), 7.42-44 (2H, m), 8.00-8.02 (1H, m), 8.54 (1H, |
| | s). |
| 121 | (DMSO-d6) 1.15-1.28 (5H, m), 1.53-1.70 (2H, m), 2.16 (3H, s), |
| | 2.38-2.46 (2H, m), 2.55-2.62 (1H, m), 3.73 (1H, d, J = 14.5 Hz), 3.99-4.14 (2H, |
| | m), 4.23 (1H, d, J = 13.2 Hz), 5.34 (1H, d, J = 14.5 Hz), 6.40 (1H, d, J = 5.0 Hz), |
| | 6.56 (1H, s), 7.05 (1H, dd, J = 2.3, 8.4 Hz), 7.11 (1H, s), 7.32 (1H, |
| | d, J = 8.4 Hz), 7.44 (1H, d, J = 2.2 Hz), 7.89 (1H, d, J = 5.0 Hz), 8.54 (1H, s). |
| 122 | (DMSO-d6) 1.23 (3H, t, J = 7.1 Hz), 1.20-1.27 (2H, m), 1.53-1.70 (2H, m), |
| | 2.09 (3H, s), 2.35-2.43 (2H, m), 2.55 (1H, dt, J = 2.8, 12.7 Hz), 3.73 (1H, d, |
| | J = 14.3 Hz), 3.99-4.12 (3H, m), 4.16 (1H, d, J = 13.2 Hz), 5.34 (1H, d, |
| | J = 14.3 Hz), 6.66 (1H, d, J = 8.9 Hz), 7.04 (1H, dd, J = 2.5, 8.4 Hz), 7.10 (1H, |
| | s), 7.28-7.33 (2H, m), 7.43 (1H, d, J = 2.5 Hz), 7.87 (1H, d, J = 2.5 Hz), |
| | 8.53 (1H, s). |
| 123 | (DMSO-d6): 1.28-1.36 (1H, m), 1.55-1.74 (1H, m), 2.09 (3H, s), |
| | 2.37-2.48 (1H, m), 2.52-2.63 (1H, m), 3.40-3.52 (2H, m), 3.83 (1H, d, |
| | J = 14.6 Hz), 4.05 (1H, d, J = 14.0 Hz), 4.17 (1H, d, J = 14.0 Hz), 5.39 (1H, d, |
| | J = 14.6 Hz), 6.66 (1H, d, J = 8.7 Hz), 7.03 (1H, dd, J = 2.4, 8.5 Hz), |
| | 7.24-7.35 (2H, m), 7.36-7.52 (6H, m), 7.54 (1H, d, J = 8.5 Hz), 7.87 (1H, d, J = 2.1 Hz), |
| | 8.54 (1H, s) |
| 124 | (DMSO-d6) 1.24-1.29 (2H, m), 1.52-1.62 (1H, m), 1.65-1.71 (1H, m), |
| | 2.24 (3H, s), 2.31 (3H, s), 2.32-2.46 (2H, m), 2.49-2.55 (1H, m), |
| | 3.68 (3H, s), 3.69 (1H, d, J = 14.5 Hz), 4.10 (1H, d, J = 13.1 Hz), 4.24 (1H, d, |
| | J = 13.1 Hz), 5.35 (1H, d, J = 14.5 Hz), 6.41 (1H, d, J = 7.2 Hz), 6.50 (1H, d, |
| | J = 8.5 Hz), 6.8 (1H, dd, J = 1.5, 8.0 Hz), 7.06 (1H, s), 7.14 (2H, d, J = 8.0 Hz), |
| | 7.34 (1H, t, J = 8.4 Hz), 8.32 (1H, s) |
| 125 | (DMSO-d6) 1.21-1.27 (2H, m), 1.51-1.62 (1H, m), 1.68-1.73 (1H, m), |
| | 2.24 (3H, s), 2.33-2.44 (2H, m), 2.52-2.59 (1H, m), 3.68 (3H, s), |
| | 3.72 (1H, d, J = 14.5 Hz), 4.11 (1H, d, J = 13.1 Hz), 4.23 (1H, d, J = 13.1 Hz), |
| | 5.34 (1H, d, J = 14.5 Hz), 6.41 (1H, d, J = 7.1 Hz), 6.51 (1H, d, J = 8.5 Hz), |
| | 7.05 (1H, dd, J = 2.4, 8.5 Hz), 7.09 (1H, s), 7.33 (2H, d, J = 8.3 Hz), 7.44 (1H, d, |
| | J = 2.4 Hz), 8.58 (1H, s) |
| 126 | (DMSO-d6) 0.80-0.86 (2H, m), 1.24 (3H, t, J = 7.1 Hz), 1.47-1.63 (1H, m), |
| | 1.67-1.70 (1H, m), 2.24 (3H, s), 2.37-2.44 (2H, m), 2.53-2.60 (1H, m), |
| | 3.73 (1H, d, J = 14.5 Hz), 3.99-4.11 (2H, m), 4.23 (1H, d, J = 13.2 Hz), |
| | 5.34 (1H, d, J = 14.6 Hz), 6.42 (1H, d, J = 7.2 Hz), 6.51 (1H, d, J = 8.5 Hz), |
| | 7.05 (1H, dd, J = 2.3, 8.4 Hz), 7.11 (1H, s), 7.31-7.36 (2H, m), 7.44 (1H, d, J = 2.0 Hz), |
| | 8.54 (1H, s). |
| 127 | (DMSO-d6) 1.23-1.33 (1H, m), 1.47 (1H, dq, J = 3.8, 12.5 Hz), |
| | 1.75-1.85 (2H, m), 2.23-2.32 (2H, m), 2.30 (3H, s), 2.40-2.46 (1H, m), 3.14 (1H, d, |
| | J = 11.9 Hz), 3.29 (1H, d, J = 11.9 Hz), 3.68 (3H, s), 3.74 (1H, d, J = 14.5 Hz), |
| | 5.38 (1H, d, J = 14.5 Hz), 6.84 (1H, dd, J = 1.3, 8.0 Hz), 6.87-6.97 (2H, m), |
| | 7.00-7.08 (2H, m), 7.08 (1H, s), 7.13-7.17 (2H, m), 8.30 (1H, s) |
| 128 | (DMSO-d6): 1.28 (1H, dd, J = 2.8, 13.2 Hz), 1.35-1.45 (1H, m), |
| | 1.68-1.74 (2H, m), 2.18 (3H, s), 2.20-2.25 (1H, m), 2.30 (3H, s), 2.27-2.34 (1H, m), |
| | 2.36-2.43 (1H, m), 3.48 (1H, d, J = 12.4 Hz), 3.62 (1H, d, J = 12.4 Hz), |
| | 3.67 (3H, s), 3.69 (1H, d, J = 14.6 Hz), 5.37 (1H, d, J = 14.6 Hz), 6.52 (1H, d, |
| | J = 7.3 Hz), 6.60-6.66 (2H, m), 6.83 (1H, dd, J = 1.3, 7.3 Hz), 6.98-7.03 (1H, |
| | m), 7.06 (1H, s), 7.12-7.16 (2H, m), 8.30 (1H, s). |
| 129 | (DMSO-d6): 1.23-1.31 (1H, m), 1.36-1.47 (1H, m), 1.67-1.75 (2H, m), |
| | 2.15 (3H, s), 2.13-2.18 (1H, m), 2.23-2.40 (2H, m), 2.30 (3H, s), |
| | 3.41 (1H, d, J = 12.6 Hz), 3.55 (1H, d, J = 12.6 Hz), 3.67 (3H, s), 3.69 (1H, d, |
| | J = 14.5 Hz), 5.37 (1H, d, J = 14.5 Hz), 6.72 (2H, d, J = 8.4 Hz), 6.80-6.84 (1H, |
| | m), 6.94 (2H, d, J = 8.4 Hz), 7.06 (1H, s), 7.1-7.16 (2H, m), 8.30 (1H, s) |
| 130 | (DMSO-d6) 1.28 (1H, dd, J = 3.1, 13.1 Hz), 1.34-1.44 (1H, m), |
| | 1.68-1.74 (2H, m), 2.19-2.36 (2H, m), 2.30 (3H, s), 2.37-2.45 (1H, m), 3.50 (1H, d, |
| | J = 13.1 Hz), 3.64 (1H, d, J = 13.1 Hz), 3.66 (3H, s), 3.67 (3H, s), 3.70 (1H, |
| | d, J = 14.3 Hz), 5.37 (1H, d, J = 14.3 Hz), 6.29 (1H, dd, J = 2.2, 7.8 Hz), |
| | 6.33-6.35 (1H, m), 6.42 (1H, dd, J = 2.2, 8.1 Hz), 6.83 (1H, dd, J = 1.2, 7.8 Hz), |
| | 7.00-7.06 (2H, m), 7.13-7.16 (2H, m), 8.30 (1H, s). |
| 131 | (DMSO-d6): 1.29 (1H, dd, J = 2.5, 12.8 Hz), 1.39-1.49 (1H, m), |
| | 1.70-1.77 (2H, m), 2.11 (1H, dt, J = 2.5, 11.9 Hz), 2.21-2.33 (2H, m), 2.30 (3H, s), |
| | 3.30 (1H, d, J = 11.7 Hz), 3.44 (1H, d, J = 11.7 Hz), 3.64 (3H, s), 3.67 (3H, |
| | s), 3.70 (1H, d, J = 14.3 Hz), 5.38 (1H, d, J = 14.3 Hz), 6.72-6.84 (5H, m), |
| | 7.06 (1H, s), 7.12-7.15 (2H, m), 8.29 (1H, s). |
| 132 | (DMSO-d6) 1.09 (3H, t, J = 7.5 Hz), 1.29 (1H, dd, J = 2.5, 13.2 Hz), |
| | 1.36-1.47 (1H, m), 1.70-1.75 (2H, m), 2.16 (1H, dt, J = 2.5, 12.4 Hz), |
| | 2.24-2.39 (2H, m), 2.30 (3H, s), 2.45 (2H, q, J = 7.5 Hz), 3.43 (1H, d, J = 12.4 Hz), |
| | 3.56 (1H, d, J = 12.4 Hz), 3.67 (3H, s), 3.69 (1H, d, J = 14.6 Hz), 5.37 (1H, d, |
| | J = 14.6 Hz), 6.75 (2H, d, J = 8.5 Hz), 6.82 (1H, d, J = 8.0 Hz), 6.97 (2H, d, |
| | J = 8.5 Hz), 7.06 (1H, s), 7.12-7.15 (2H, m), 8.30 (1H, s). |
| 133 | (DMSO-d6) 1.27-1.41 (2H, m), 1.63-1.72 (2H, m), 2.30 (3H, s), |
| | 2.39-2.46 (2H, m), 2.57-2.64 (1H, m), 3.68 (3H, s), 3.69-3.71 (2H, m), |
| | 3.82 (1H, d, J = 13.2 Hz), 5.37 (1H, d, J = 14.4 Hz), 6.83 (1H, dd, J = 1.3, 8.0 Hz), |
| | 6.95 (2H, d, J = 8.8 Hz), 7.06 (1H, s), 7.14-7.16 (2H, m), 7.42 (2H, d, |
| | J = 8.8 Hz), 8.30 (1H, s). |
| 134 | (DMSO-d6) 1.23-1.33 (1H, m), 1.47 (1H, dq, J = 3.8, 12.5 Hz), |
| | 1.75-1.85 (2H, m), 2.23-2.32 (2H, m), 2.40-2.48 (1H, m), 3.14 (1H, d, J = 11.9 Hz), |
| | 3.29 (1H, d, J = 11.9 Hz), 3.68 (3H, s), 3.74 (1H, d, J = 14.5 Hz), 5.38 (1H, d, |
| | J = 14.5 Hz), 6.87-6.97 (2H, m), 7.00-7.08 (2H, m), 7.11 (1H, s), 7.32 (1H, |
| | d, J = 8.4 Hz), 7.43 (1H, d, J = 2.4 Hz), 8.56 (1H, s) |
| 135 | (DMSO-d6) 1.26 (1H, dd, J = 2.3, 13.2 Hz), 1.34-1.44 (1H, m), |
| | 1.69-1.74 (2H, m), 2.19 (3H, s), 2.22-2.33 (2H, m), 2.40-2.47 (1H, m), 3.48 (1H, d, |
| | J = 12.5 Hz), 3.63 (1H, d, J = 12.5 Hz), 3.68 (3H, s), 3.73 (1H, d, J = 14.6 Hz), |
| | 5.36 (1H, d, J = 14.6 Hz), 6.52 (1H, d, J = 7.3 Hz), 6.61-6.66 (2H, m), |
| | 6.99-7.05 (2H, m), 7.09 (1H, s), 7.32 (1H, d, J = 8.3 Hz), 7.43 (1H, d, J = 2.5 Hz), |
| | 8.56 (1H, s). |
| 136 | (DMSO-d6) 1.23-1.29 (1H, m), 1.35-1.45 (1H, m), 1.71-1.78 (2H, m), |
| | 2.15 (3H, s), 2.19-2.31 (2H, m), 2.32-2.42 (1H, m), 3.41 (1H, d, |
| | J = 13.0 Hz), 3.54 (1H, d, J = 13.0 Hz), 3.68 (3H, s), 3.72 (1H, d, J = 14.5 Hz), |
| | 5.36 (1H, d, J = 14.5 Hz), 6.73 (2H, d, J = 8.5 Hz), 6.94 (2H, d, J = 8.5 Hz), |
| | 7.03 (1H, dd, J = 1.3, 8.4 Hz), 7.10 (1H, s), 7.31 (1H, d, J = 8.4 Hz), |
| | 7.43 (1H, d, J = 2.3 Hz), 8.57 (1H, s) |
| 137 | (DMSO-d6) 1.26 (1H, dd, J = 3.1, 13.1 Hz), 1.32-1.43 (1H, m), |
| | 1.69-1.74 (2H, m), 2.24-2.34 (2H, m), 2.42-2.49 (1H, m), 3.50 (1H, d, J = 12.5 Hz), |
| | 3.65 (1H, d, J = 12.5 Hz), 3.66 (3H, s), 3.68 (3H, s), 3.73 (1H, d, |
| | J = 14.6 Hz), 5.36 (1H, d, J = 14.6 Hz), 6.29 (1H, dd, J = 2.2, 7.8 Hz), |
| | 6.33-6.35 (1H, m), 6.42 (1H, dd, J = 2.2, 8.1 Hz), 7.00-7.05 (2H, m), 7.10 (1H, |
| | s), 7.32 (1H, d, J = 7.8 Hz), 7.43 (1H, d, J = 2.2 Hz), 8.57 (1H, s). |
| 138 | (DMSO-d6) 1.27 (1H, dd, J = 2.5, 12.9 Hz), 1.37-1.48 (1H, m), |
| | 1.72-1.77 (2H, m), 2.16 (1H, dt, J = 2.5, 12.3 Hz), 2.20-2.27 (1H, m), 2.30-2.39 (1H, |
| | m), 3.31 (1H, d, J = 11.7 Hz), 3.45 (1H, d, J = 11.7 Hz), 3.64 (3H, s), |
| | 3.68 (3H, s), 3.74 (1H, d, J = 14.3 Hz), 5.37 (1H, d, J = 14.3 Hz), 6.74 (2H, d, |
| | J = 9.2 Hz), 6.79 (2H, d, J = 9.2 Hz), 7.04 (1H, dd, J = 2.2, 8.4 Hz), 7.09 (1H, |
| | s), 7.32 (1H, d, J = 8.4 Hz), 7.42 (1H, d, J = 2.2 Hz), 8.57 (1H, s). |
| 139 | (DMSO-d6) 1.09 (3H, t, J = 7.9 Hz), 1.26 (1H, dd, J = 2.5, 12.5 Hz), |
| | 1.35-1.45 (1H, m), 1.70-1.76 (2H, m), 2.18-2.31 (2H, m), 2.36-2.47 (1H, m), |
| | 2.45 (2H, q, J = 7.9 Hz), 3.43 (1H, d, J = 12.4 Hz), 3.57 (1H, d, J = 12.4 Hz), |
| | 3.68 (3H, s), 3.73 (1H, d, J = 14.3 Hz), 5.36 (1H, d, J = 14.3 Hz), 6.75 (2H, d, |
| | J = 8.5 Hz), 6.98 (2H, d, J = 8.5 Hz), 7.04 (1H, dd, J = 2.2, 8.5 Hz), 7.09 (1H, |
| | s), 7.32 (1H, d, J = 8.5 Hz), 7.43 (1H, d, J = 2.2 Hz), 8.56 (1H, s). |
| 140 | (DMSO-d6) 1.25-1.40 (2H, m), 1.63-1.75 (2H, m), 2.37-2.45 (2H, m), |
| | 2.61-2.68 (1H, m), 3.68 (3H, s), 3.70-3.79 (2H, m), 3.83 (1H, d, |
| | J = 13.1 Hz), 5.36 (1H, d, J = 14.5 Hz), 6.96 (2H, d, J = 8.8 Hz), 7.05 (1H, dd, |
| | J = 2.4, 8.4 Hz), 7.09 (1H, s), 7.33 (1H, d, J = 8.4 Hz), 7.41 (1H, s), |
| | 7.43-7.45 (2H, m), 8.57 (1H, s). |
| 141 | (DMSO-d6) 1.23-1.32 (1H, m), 1.37-1.46 (1H, m), 1.66-1.78 (2H, m), |
| | 2.14-2.29 (2H, m), 2.30 (6H, s), 2.36-2.48 (1H, m), 3.47 (1H, d, |
| | J = 12.4 Hz), 3.62 (1H, d, J = 12.4 Hz), 3.67 (3H, s), 3.68 (1H, d, J = 14.5 Hz), |
| | 5.37 (1H, d, J = 14.5 Hz), 6.82 (1H, dd, J = 1.3, 8.1 Hz), 6.99 (1H, d, |
| | J = 8.6 Hz), 7.07 (1H, s), 7.13 (1H, d, J = 2.8 Hz), 7.15 (2H, d, J = 1.5 Hz), |
| | 8.04 (1H, d, J = 2.8 Hz), 8.32 (1H, s) |
| 142 | (DMSO-d6) 1.23-1.30 (1H, m), 1.33-1.45 (1H, m), 1.70-1.77 (2H, m), |
| | 2.23-2.35 (2H, m), 2.30 (3H, s), 2.41-2.49 (1H, m), 3.47 (1H, d, |
| | J = 12.4 Hz), 3.61 (1H, d, J = 12.4 Hz), 3.68 (3H, s), 3.73 (1H, d, J = 14.5 Hz), |
| | 5.35 (1H, d, J = 14.5 Hz), 6.99 (1H, d, J = 8.5 Hz), 7.04 (1H, dd, J = 2.3, |
| | 8.4 Hz), 7.10 (1H, s), 7.14 (1H, dd, J = 3.0, 8.6 Hz), 7.33 (1H, d, J = 8.4 Hz), |
| | 7.43 (1H, d, J = 2.3 Hz), 8.05 (1H, d, J = 3.0 Hz), 8.58 (1H, s) |
| 143 | 1.35-1.60 (1H, m), 1.60-2.10 (3H, m), 2.45-2.85 (3H, m), 4.13 (1H, d, |
| | J = 16.1 Hz), 4.55-4.85 (4H, m), 5.26 (1H, d, J = 13.6 Hz), 5.85-6.15 (3H, |
| | m), 6.42 (1H, t, J = 4.7 Hz), 6.58 (1H, m), 7.20-7.55 (4H, m), 8.25 (1H, d, |
| | J = 4.7 Hz) |
| 144 | 1.40-1.55 (1H, m), 1.71-2.11 (3H, m), 2.42-2.75 (2H, m), 4.06-4.35 (3H, |
| | m), 4.71 (1H, d, J = 16.2 Hz), 5.26 (1H, d, J = 16.2 Hz), 5.90-6.02 (2H, m), |
| | 6.05 (1H, s), 6.50-6.62 (3H, m), 7.27-7.30 (2H, m), 7.31-7.47 (4H, m), |
| | 8.12 (1H, d, J = 3.9 Hz) |
| 145 | (MeOH, D3) 1.25-1.35 (1H, m), 1.50-1.54 (1H, m), 1.75-1.91 (4H, m), |
| | 2.15 (3H, s), 2.46-2.63 (2H, m), 4.04-4.19 (3H, m), 4.90 (1H, d, J = 14.1 Hz), |
| | 5.18 (1H, d, J = 14.1 Hz), 5.77 (1H, d, J = 16.1 Hz), 5.90-5.97 (2H, m), |
| | 6.65-6.66 (1H, m), 6.72 (1H, d, J = 8.7 Hz), 7.42-7.54 (4H, m), 7.86 (1H, |
| | brs). |
| 146 | 1.48-1.58 (1H, m), 1.86-2.21 (3H, m), 2.37-2.66 (3H, m), 3.53-3.75 (2H, |
| | m), 4.09-4.18 (1H, m), 4.65-4.75 (1H, m), 5.21-5.32 (1H, m), 5.98 (2H, t, |
| | J = 7.4 Hz), 6.06 (1H, t, J = 3.0 Hz), 6.59 (1H, t, J = 1.9 Hz), 7.08-7.15 (2H, |
| | m), 7.26-7.32 (1H, m), 7.33-7.49 (3H, m), 8.04 (1H, t, J = 2.7 Hz), |
| | 8.22 (1H, s) |
| 147 | 1.35-1.48 (1H, m), 1.49-1.61 (1H, m), 1.77-2.18 (4H, m), 2.66-2.80 (1H, |
| | m), 2.95-3.22 (3H, m), 3.90-4.10 (2H, m), 4.16 (1H, d, J = 8.1 Hz), |
| | 4.74, (1H, d, J = 8.1 Hz), 5.22 (1H, d, J = 8.1 Hz), 5.85-6.00 (1H, m), 6.08 (1H, s), |
| | 6.55-6.63 (1H, m), 6.66-6.82 (1H, m), 7.28-7.32 (1H, m), 7.33-7.50 (3H, |
| | m), 8.02-8.20 (1H, m) |
| 148 | 1.43-1.58 (1H, m), 1.71-2.07 (4H, m), 2.50-2.81 (3H, m), 4.05-4.39 (3H, |
| | m), 4.71 (1H, d, J = 13.9 Hz), 5.25 (1H, d, J = 13.9 Hz), 5.91-6.00 (2H, m), |
| | 6.04 (1H, t, J = 3.0 Hz), 6.58 (1H, s), 7.25-7.32 (1H, m), 7.33-7.47 (2H, m), |
| | 7.77 (1H, d, J = 2.7 Hz), 7.96-8.01 (1H, m), 8.07 (1H, s) |
| 149 | 1.42-1.58 (2H, m), 1.79-2.22 (3H, m), 2.32-2.61 (3H, m), 3.52-3.73 (2H, |
| | m), 4.13 (1H, d, J = 16 Hz), 4.71 (1H, d, J = 13.9 Hz), 5.26 (1H, d, |
| | J = 12.9 Hz), 5.88-6.11 (3H, m), 6.58 (1H, s), 6.75-6.90 (3H, m), |
| | 7.12-7.50 (5H, m) |
| 150 | 1.35-1.55 (1H, m), 1.60-2.15 (3H, m), 2.40-2.80 (3H, m), 4.00-4.40 (2H, |
| | m), 4.13 (1H, d, J = 16.3 Hz), 4.65 (1H, d, J = 14.1 Hz), 5.22 (1H, d, |
| | J = 14.1 Hz), 5.87-6.02 (2H, m), 6.06 (1H, t, J = 3.2 Hz), 6.51-6.68 (3H, m), |
| | 7.04-7.22 (2H, m), 7.22-7.34 (1H, m), 7.37-7.50 (1H, m), 8.09-8.20 (1H, |
| | m). |
| 151 | 1.15-1.37 (1H, m), 1.40-1.70 (2H, m), 1.75-2.20 (3H, m), 2.28-2.62 (2H, |
| | m), 4.50-4.80 (2H, m), 4.13 (1H, d, J = 16.1 Hz), 4.65 (1H, d, J = 13.9 Hz), |
| | 5.23 (1H, d, J = 13.9 Hz), 5.87-6.02 (2H, m), 6.06 (1H, t, J = 3.2 Hz), |
| | 6.53-6.64 (1H, m), 6.76-6.98 (1H, m), 6.85 (2H, d, J = 8.7 Hz), 7.03-7.40 (4H, |
| | m). |
| 152 | 1.48-1.55 (2H, m), 1.79-1.99 (3H, m), 2.38 (3H, s), 2.51-2.67 (3H, m), |
| | 4.11 (1H, d, J = 15.8 Hz), 4.23-4.29 (2H, m), 4.63 (1H, d, J = 13.9 Hz), |
| | 5.22 (1H, d, J = 13.6 Hz), 5.91-5.97 (2H, m), 6.03 (1H, s), 6.52-6.60 (3H, m), |
| | 7.13-7.24 (2H, m), 7.42 (1H, t, J = 7.2 Hz), 8.12 (1H, d, J = 4.2 Hz). |
| 153 | 1.80-2.10 (4H, m), 2.37 (3H, s), 2.41-2.52 (4H, m), 3.59-3.69 (2H, m), |
| | 4.11 (1H, d, J = 16.6 Hz), 4.63 (1H, d, J = 13.9 Hz), 5.23 (1H, d, J = 13.9 Hz), |
| | 5.95-5.99 (2H, m), 6.04 (1H, t, J = 3.2 Hz), 6.56 (1H, s), |
| | 6.77-6.86 (3H, m), 7.13-7.24 (4H, m). |
| 154 | 1.40-1.60 (1H, m), 1.78-2.28 (3H, m), 2.30-2.60 (3H, m), 3.35 (1H, d, |
| | J = 11.6 Hz), 3.45 (1H, d, J = 11.6 Hz), 4.14 (1H, d, J = 15.8 Hz), 4.70 (1H, d, |
| | J = 15.8 Hz), 5.17-5.36 (1H, m), 5.90-6.12 (3H, m), 6.58 (1H, m), |
| | 6.76-7.10 (4H, m), 7.22-7.52 (4H, m). |
| 155 | 1.40-1.70 (1H, m), 1.70-2.20 (3H, m), 2.25-3.65 (3H, m), 3.57 (1H, d, |
| | J = 12.3 Hz), 3.68 (1H, d, J = 12.4 Hz), 4.15 (1H, d, J = 16.1 Hz), 4.71 (1H, d, |
| | J = 12.4 Hz), 5.26 (1H, d, J = 12.4 Hz), 5.86-6.14 (2H, m), 6.06 (1H, t, |
| | J = 3.0 Hz), 6.40-6.60 (4H, m), 7.13 (1H, quartet, J = 7.9 Hz), 7.22-7.52 (4H, |
| | m). |
| 156 | 1.40-1.70 (1H, m), 1.75-2.20 (3H, m), 2.22-2.55 (3H, m), 3.44 (1H, d, |
| | J = 11.4 Hz), 3.54 (1H, d, J = 11.4 Hz), 4.14 (1H, d, J = 16.1 Hz), 4.71 (1H, d, |
| | J = 13.9 Hz), 5.26 (1H, d, J = 13.9 Hz), 5.90-6.10 (2H, m), 6.06 (1H, t, |
| | J = 3.0 Hz), 6.58 (1H, m), 6.72-6.98 (4H, m), 7.22-7.50 (4H, m). |
| 157 | 1.40-1.62 (1H, m),, 1.75-1.85 (1H, m), 1.85-2.58 (5H, m), 3.29 (1H, d, |
| | J = 10.6 Hz), 3.38 (1H, d, J = 10.6 Hz), 4.15 (1H, d, J = 16.6 Hz), 4.71 (1H, d, |
| | J = 13.9 Hz), 5.28 (1H, d, J = 13.9 Hz), 5.90-6.12 (3H, m), 6.58 (1H, s), |
| | 6.82-6.98 (2H, m), 7.07-7.20 (1H, m), 7.22-7.52 (5H, m). |
| 158 | 1.38-1.70 (1H, m), 1.75-2.20 (3H, m), 2.25-2.60 (3H, m), 3.51 (1H, d, |
| | J = 11.4 Hz), 3.62 (1H, d, J = 11.4 Hz), 4.14 (1H, d, J = 16.3 Hz), 4.71 (1H, d, |
| | J = 13.9 Hz), 5.26 (1H, d, J = 13.9 Hz), 5.90-6.14 (2H, m), 6.06 (1H, t, |
| | J = 3.0 Hz), 6.58 (1H, s), 6.76 (2H, d, J = 8.9 Hz), 7.14 (2H, d, J = 8.9 Hz), |
| | 7.22-7.52 (4H, m). |
| 159 | 1.40-1.60 (1H, m), 1.80-2.55 (6H, m), 2.27 (3H, s), 3.02 (1H, d, |
| | J = 10.6 Hz), 3.12 (1H, d, J = 10.6 Hz), 4.16 (1H, d, J = 15.6 Hz), 4.71 (1H, d, |
| | J = 13.9 Hz), 5.28 (1H, d, J = 13.9 Hz), 5.92-6.15 (3H, m), 6.59 (1H, s), |
| | 6.80-7.03 (2H, m), 7.03-7.22 (2H, m), 7.22-7.52 (4H, m). |
| 160 | 1.56 (2H, m), 1.90-2.20 (4H, m), 2.30 (3H, s), 2.36-2.45 (2H, m), |
| | 3.59-3.68 (2H, m), 4.13 (1H, d, J = 16.6 Hz), 4.73 (1H, d, J = 13.6 Hz), 5.29 (1H, |
| | d, J = 13.6 Hz), 5.97 (2H, m), 6.05 (1H, t, J = 3.2 Hz), 6.58-6.67 (4H, m), |
| | 7.10 (1H, t, J = 7.9 Hz), 7.25-7.42 (3H, m). |
| 161 | 1.53 (1H, m), 1.90-2.10 (4H, m), 2.28 (3H, s), 2.34-2.45 (3H, m), |
| | 3.53-3.61 (2H, m), 4.14 (1H, d, J = 16.1 Hz), 4.70 (1H, d, J = 13.9 Hz), 5.27 (1H, |
| | d, J = 13.9 Hz), 5.97-6.01 (2H, m), 6.05 (1H, t, J = 3.2 Hz), 6.58 (1H, s), |
| | 6.76 (2H, d, J = 8.4 Hz), 7.03 (2H, d, J = 8.2 Hz), 7.27-7.30 (1H, m), |
| | 7.37-7.42 (2H, m). |
| 162 | 1.40-1.60 (1H, m), 1.78-1.94 (1H, m), 1.94-2.50 (5H, m), 3.30-3.60 (2H, |
| | m), 3.84 (3H, s), 4.14 (1H, d, J = 16.1 Hz), 4.70 (1H, d, J = 13.6 Hz), |
| | 5.28 (1H, d, J = 13.6 Hz), 5.90-6.12 (3H, m), 6.58 (1H, m), 6.74-7.04 (4H, m), |
| | 7.20-7.50 (4H, m). |
| 163 | 1.42-1.65 (1H, m), 1.75-2.22 (3H, m), 2.30-2.60 (3H, m), 3.57 (1H, d, |
| | J = 12.6 Hz), 3.68 (1H, d, J = 12.6 Hz), 3.75 (3H, s), 4.14 (1H, d, J = 16.1 Hz), |
| | 4.70 (1H, d, J = 13.6 Hz), 5.26 (1H, d, J = 13.6 Hz), 5.90-6.14 (2H, m), |
| | 6.05 (1H, t, J = 3.1 Hz), 6.31-6.42 (2H, m), 6.42-6.52 (1H, m), 6.58 (1H, m), |
| | 7.05-7.18 (1H, m), 7.20-7.52 (4H, m). |
| 164 | (CD₃OD) 1.47-1.65 (1H, m), 1.70-2.12 (3H, m), 2.15-2.52 (3H, m), |
| | 3.35 (1H, d, J = 11.4 Hz), 3.47 (1H, d, J = 11.4 Hz), 3.70 (3H, s), 4.13 (1H, d, |
| | J = 16.1 Hz), 4.82-5.00 (1H, m), 5.18 (1H, d, J = 14.1 Hz), 5.82 (1H, d, |
| | J = 16.1 Hz), 5.88-6.06 (2H, m), 6.65 (1H, m), 6.78 (2H, d, J = 9.2 Hz), |
| | 6.87 (2H, d, J = 9.2 Hz), 7.34-7.60 (4H, m). |
| 165 | 1.17 (3H, t J = 7.4 Hz), 1.40-1.59 (1H, m), 1.80-2.20 (3H, m), |
| | 2.27-2.43 (3H, m), 2.44-2.61 (2H, m), 3.43-3.72 (2H, m), 4.14 (1H, d, J = 15.8 Hz), |
| | 4.70 (1H, d, J = 14.4 Hz), 5.27 (1H, d, J = 14.4 Hz), 5.92-6.03 (2H, m), |
| | 6.04-6.11 (1H, m), 6.58 (1H, s), 6.78 (2H, d J = 8.4 Hz), 7.04 (2H, d J = 8.4 Hz), |
| | 7.26-7.31 (1H, m), 7.32-7.50 (3H, m) |
| 166 | 0.89 (3H, t, J = 7.4 Hz), 1.43-1.67 (3H, m), 1.73-2.20 (3H, m), |
| | 2.27-2.56 (5H, m), 3.45-3.70 (1H, m), 4.14 (1H, d, J = 14.8 Hz), 4.71 (1H, d, |
| | J = 15.1 Hz), 5.27 (1H, d, J = 14.8 Hz), 5.90-6.00 (2H, m), 6.01-6.11 (1H, |
| | m), 6.58 (1H, s), 6.78 (2H, d, J = 8.5 Hz), 7.00 (2H, d, J = 8.5 Hz), |
| | 7.26-7.31 (2H, m), 7.32-7.47 (3H, m) |
| 167 | 1.17 (6H, d, J = 6.9 Hz), 1.40-1.60 (1H, m), 1.78-2.20 (3H, m), |
| | 2.27-2.53 (3H, m), 2.71-2.86 (1H, m), 3.42-3.70 (2H, m), 4.12 (1H, d, J = 16.8 Hz), |
| | 4.69 (1H, d, J = 13.9 Hz), 5.25 (1H, d, J = 13.9 Hz), 5.90-5.98 (2H, m), |
| | 5.99-6.08 (1H, m), 6.57 (1H, s), 6.77 (2H, d, J = 8.4 Hz), 7.05 (2H, d, J = 8.4 Hz), |
| | 7.26-7.30 (1H, m), 7.31-7.47 (3H, m) |
| 168 | (DMSO-d6) 1.52-2.07 (4H, m), 2.39-2.44 (2H, m), 3.67-3.80 (2H, m), |
| | 4.07 (1H, d, J = 15.8 Hz), 5.10 (1H, d, J = 15.1 Hz), 5.80 (1H, d, J = 16.1 Hz), |
| | 5.90 (2H, d, J = 11.4 Hz), 6.74 (1H, s), 6.87-7.62 (15H, m). |
| 169 | (DMSO-d6) 1.43-1.89 (4H, m), 2.60-2.71 (2H, m), 3.71-3.92 (2H, m), |
| | 4.06 (1H, d, J = 16.3 Hz), 5.07 (2H, q, J = 13.1 Hz), 5.75 (1H, d, J = 16.3 Hz), |
| | 5.87 (1H, s), 5.92 (1H, t, J = 3.2 Hz), 6.74 (1H, t, J = 2.0 Hz), 6.99 (2H, d, |
| | J = 8.7 Hz), 7.43 (2H, d, J = 8.7 Hz), 7.48-7.53 (2H, m), 7.54-7.65 (2H, m) |
| 170 | (DMSO-d6) 1.51-1.63 (1H, m), 1.70-2.00 (3H, m), 2.30-2.61 (4H, m), |
| | 3.41-3.52 (1H, m), 4.08 (1H, d, J = 16.3 Hz), 5.12 (2H, q, J = 13.1 Hz), |
| | 5.77 (1H, d, J = 16.3 Hz), 5.91 (2H, d, J = 9.4 Hz), 6.74 (1H, s), 7.05 (2H, t, |
| | J = 8.2 Hz), 7.40 (1H, d, J = 7.4 Hz), 7.43-7.62 (4H, m), 7.65 (1H, d, |
| | J = 7.4 Hz) |
| 171 | (DMSO-d6) d 1.34-1.57 (4H, m), 2.22-2.25 (4H, m), 3.40-3.51 (2H, m), |
| | 3.79 (1H, d, J = 15.1 Hz), 4.78-4.91 (2H, m), 5.47 (1H, d, J = 16.1 Hz), |
| | 5.64 (2H, d, J = 11.6 Hz), 6.46 (1H, s), 6.83-7.26 (7H, m). |
| 172 | 1.50-1.60 (1H, m), 1.75-2.15 (6H, m), 2.63-2.45 (3H, m), 3.62-3.67 (2H, |
| | m), 3.74 (2H, brs), 4.14 (1H, d, J = 15.8 Hz), 4.71 (1H, d, J = 13.9 Hz), (1H, |
| | d, J = 13.9 Hz), 5.97 (2H, brs), 6.05 (1H, t, J = 3.5 Hz), 6.58 (1H, s), |
| | 6.82 (2H, d, J = 8.7 Hz), 7.16 (2H, d, J = 8.4 Hz), 7.25-7.30 (1H, m), |
| | 7.40-7.42 (2H, m). |
| 173 | (DMSO-d6) 1.39-1.48 (1H, m), 1.61-1.92 (3H, m), 2.29-2.41 (1H, m), |
| | 2.42-2.58 (3H, m), 2.99-3.22 (2H, m), 4.07 (1H, d, J = 16.8 Hz), 5.10 (2H, |
| | q, J = 13.9 Hz), 5.80 (1H, d, J = 16.8 Hz), 5.90-5.99 (2H, m), 6.72 (1H, t, |
| | J = 2.1 Hz), 7.04-7.12 (1H, m), 7.24 (1H, d, 7.9 Hz), 7.39-7.62 (4H, m), |
| | 7.76 (1H, dd, J = 1.5 Hz, 8.2 Hz) |
| 174 | 1.52-1.88 (4H, m), 2.50-2.70 (4H, m), 3.82-3.87 (2H, m), 4.06 (1H, d, |
| | J = 15.8 Hz), 4.90-5.18 (2H, m), 5.76 (1H, d, J = 16.1 Hz), 5.90-5.93 (2H, |
| | m), 6.73 (1H, s), 7.42-7.58 (7H, m). |
| 176 | 1.89-2.03 (4H, m), 2.36-2.43 (3H, m), 3.02-3.16 (2H, m), 3.95 (2H, brs), |
| | 4.10-4.18 (1H, m), 4.71 (1H, d, J = 13.8 Hz), 5.27 (1H, d, J = 13.8 Hz), |
| | 5.98-6.06 (3H, m), 6.58-6.70 (4H, m), 6.82-6.88 (2H, m), 7.25-7.44 (3H, |
| | m) |
| 178 | 1.48-1.52 (1H, m), 1.89-1.99 (3H, m), 2.36-2.49 (3H, m), 2.98-3.11 (4H, |
| | m), 3.93 (2H, s), 4.13 (1H, d, J = 16.1 Hz), 4.69 (1H, d, =13.6 Hz), |
| | 5.26 (1H, d, J = 13.6 Hz), 5.93-5.97 (2H, m), 6.05 (1H, t, J = 3.0 Hz), 6.57 (1H, |
| | s), 7.04-7.43 (8H, m) |

TABLE 18
Names of Examples

| Example | Name |
|---|---|
| 6 | (2-Methyl-4,5-dihydro-1H-1,3,6-triaza-benzo[e]azulen-6-yl)-(1- |
| | pyrimidin-2-yl-piperidin-4-yl)-methanone |
| 7 | (9-Chloro-2-methyl-4,5-dihydro-1H-1,3,6-triaza-benzo[e]azulen-6-yl)-(1- |
| | pyrimidin-2-yl-piperidin-4-yl)-methanone |
| 8 | (2-Methyl-4,5-dihydro-1H-1,3,6-triaza-benzo[e]azulen-6-yl)-(3,4,5,6- |
| | tetrahydro-2H-[1,2′]bipyridinyl-4-yl)-methanone |
| 9 | (2-Morpholin-4-yl-4,5-dihydro-3H-1,3,6-triaza-benzo[e]azulen-6-yl)- |
| | (3,4,5,6-tetrahydro-2H-[1,2′]bipyridinyl-4-yl)-methanone |
| 10 | (3′-Methyl-3,4,5,6-tetrahydro-2H-[1,2′]bipyridinyl-4-yl)-(2-morpholin-4- |
| | yl-4,5-dihydro-1H-1,3,6-triaza-benzo[e]azulen-6-yl)-methanone |
| 11 | (4′-Methyl-3,4,5,6-tetrahydro-2H-[1,2′]bipyridinyl-4-yl)-(2-morpholin-4- |
| | yl-4,5-dihydro-3H-1,3,6-triaza-benzo[e]azulen-6-yl)-methanone |
| 12 | (5′-Methyl-3,4,5,6-tetrahydro-2H-[1,2′]bipyridinyl-4-yl)-(2-morpholin-4- |
| | yl-4,5-dihydro-3H-1,3,6-triaza-benzo[e]azulen-6-yl)-methanone |
| 13 | (9-Fluoro-2-morpholin-4-yl-4,5-dihydro-1H-1,3,6-triaza-benzo[e]azulen- |
| | 6-yl)-(3,4,5,6-tetrahydro-2H-[1,2′]bipyridinyl-4-yl)-methanone |
| 14 | (9-Fluoro-2-morpholin-4-yl-4,5-dihydro-1H-1,3,6-triaza-benzo[e]azulen- |
| | 6-yl)-(3′-methyl-3,4,5,6-tetrahydro-2H-[1,2′]bipyridinyl-4-yl)-methanone |
| 15 | (9-Fluoro-2-morpholin-4-yl-4,5-dihydro-1H-1,3,6-triaza-benzo[e]azulen- |
| | 6-yl)-(4′-methyl-3,4,5,6-tetrahydro-2H-[1,2′]bipyridinyl-4-yl)-methanone |
| 16 | (9-Fluoro-2-morpholin-4-yl-4,5-dihydro-1H-1,3,6-triaza-benzo[e]azulen- |
| | 6-yl)-(5′-methyl-3,4,5,6-tetrahydro-2H-[1,2′]bipyridinyl-4-yl)-methanone |
| 17 | (2-Ethyl-9-methyl-4,5-dihydro-1H-1,3,6-triaza-benzo[e]azulen-6-yl)-(3′- |
| | methyl-3,4,5,6-tetrahydro-2H-[1,2′]bipyridinyl-4-yl)-methanone |
| 18 | (2-Ethyl-9-methyl-4,5-dihydro-3H-1,3,6-triaza-benzo[e]azulen-6-yl)-(4′- |
| | methyl-3,4,5,6-tetrahydro-2H-[1,2′]bipyridinyl-4-yl)-methanone |
| 19 | (2-Ethyl-9-methyl-4,5-dihydro-1H-1,3,6-triaza-benzo[e]azulen-6-yl)-(5′- |
| | methyl-3,4,5,6-tetrahydro-2H-[1,2′]bipyridinyl-4-yl)-methanone |
| 20 | (9-Methyl-2-propyl-4,5-dihydro-3H-1,3,6-triaza-benzo[e]azulen-6-yl)- |
| | (3,4,5,6-tetrahydro-2H-[1,2′]bipyridinyl-4-yl)-methanone |
| 21 | (9-Methyl-2-propyl-4,5-dihydro-1H-1,3,6-triaza-benzo[e]azulen-6-yl)-(3′- |
| | methyl-3,4,5,6-tetrahydro-2H-[1,2′]bipyridinyl-4-yl)-methanone |
| 22 | (9-Methyl-2-propyl-4,5-dihydro-1H-1,3,6-triaza-benzo[e]azulen-6-yl)-(4′- |
| | methyl-3,4,5,6-tetrahydro-2H-[1,2′]bipyridinyl-4-yl)-methanone |
| 23 | (9-Methyl-2-propyl-4,5-dihydro-3H-1,3,6-triaza-benzo[e]azulen-6-yl)-(5′- |
| | methyl-3,4,5,6-tetrahydro-2H-[1,2′]bipyridinyl-4-yl)-methanone |
| 24 | (2-Cyclopropyl-9-methyl-4,5-dihydro-3H-1,3,6-triaza-benzo[e]azulen-6- |
| | yl)-(3,4,5,6-tetrahydro-2H-[1,2′]bipyridinyl-4-yl)-methanone |
| 25 | (2-Cyclopropyl-9-methyl-4,5-dihydro-3H-1,3,6-triaza-benzo[e]azulen-6- |
| | yl)-(5′-methyl-3,4,5,6-tetrahydro-2H-[1,2′]bipyridinyl-4-yl)-methanone |
| 26 | (2-Butyl-9-methyl-4,5-dihydro-3H-1,3,6-triaza-benzo[e]azulen-6-yl)- |
| | (3,4,5,6-tetrahydro-2H-[1,2′]bipyridinyl-4-yl)-methanone |
| 27 | (2-Butyl-9-methyl-4,5-dihydro-3H-1,3,6-triaza-benzo[e]azulen-6-yl)-(5′- |
| | methyl-3,4,5,6-tetrahydro-2H-[1,2′]bipyridinyl-4-yl)-methanone |
| 28 | (2-Isobutyl-9-methyl-4,5-dihydro-3H-1,3,6-triaza-benzo[e]azulen-6-yl)- |
| | (3,4,5,6-tetrahydro-2H-[1,2′]bipyridinyl-4-yl)-methanone |
| 29 | (2-Isobutyl-9-methyl-4,5-dihydro-3H-1,3,6-triaza-benzo[e]azulen-6-yl)- |
| | (5′-methyl-3,4,5,6-tetrahydro-2H-[1,2′]bipyridinyl-4-yl)-methanone |
| 30 | [9-Methyl-2-(4-methyl-piperazin-1-yl)-4,5-dihydro-1H-1,3,6-triaza- |
| | benzo[e]azulen-6-yl]-(5′-methyl-3,4,5,6-tetrahydro-2[1,2′]bipyridinyl-4- |
| | yl)-methanone |
| 31 | (9-Methyl-2-morpholin-4-yl-4,5-dihydro-1H-1,3,6-triaza-benzo[e]azulen- |
| | 6-yl)-(3,4,5,6-tetrahydro-2H-[1,2′]bipyridinyl-4-yl)-methanone |
| 32 | (9-Methyl-2-morpholin-4-yl-4,5-dihydro-3H-1,3,6-triaza-benzo[e]azulen- |
| | 6-yl)-(4′-methyl-3,4,5,6-tetrahydro-2H-[1,2′]bipyridinyl-4-yl)-methanone |
| 33 | (4′-Methoxy-3,4,5,6-tetrahydro-2H-[1,2′]bipyridinyl-4-yl)-(9-methyl-2- |
| | morpholin-4-yl-4,5-dihydro-1H-1,3,6-triaza-benzo[e]azulen-6-yl)- |
| | methanone |
| 34 | (5′-Methoxy-3,4,5,6-tetrahydro-2H-[1,2′]bipyridinyl-4-yl)-(9-methyl-2- |
| | morpholin-4-yl-4,5-dihydro-1H-1,3,6-triaza-benzo[e]azulen-6-yl)- |
| | methanone |
| 35 | (9-Methyl-2-morpholin-4-yl-4,5-dihydro-1H-1,3,6-triaza-benzo[e]azulen- |
| | 6-yl)-(5′-trifluoromethyl-3,4,5,6-tetrahydro-2H-[1,2′]bipyridinyl-4-yl)- |
| | methanone |
| 36 | 4-(9-Methyl-2-morpholin-4-yl-4,5-dihydro-1H-1,3,6-triaza- |
| | benzo[e]azulene-6-carbonyl)-3,4,5,6-tetrahydro-2H-[1,2′]bipyridinyl-5′- |
| | carbonitrile |
| 37 | (3′-Methyl-3,4,5,6-tetrahydro-2H-[1,2′]bipyridinyl-4-yl)-[9-methyl-2- |
| | (tetrahydro-pyran-4-yl)-4,5-dihydro-1H-1,3,6-triaza-benzo[e]azulen-6- |
| | yl]-methanone |
| 38 | (4′-Methyl-3,4,5,6-tetrahydro-2H-[1,2′]bipyridinyl-4-yl)-[9-methyl-2- |
| | (tetrahydro-pyran-4-yl)-4,5-dihydro-1H-1,3,6-triaza-benzo[e]azulen-6- |
| | yl]-methanone |
| 39 | (5′-Methyl-3,4,5,6-tetrahydro-2H-[1,2′]bipyridinyl-4-yl)-[9-methyl-2- |
| | (tetrahydro-pyran-4-yl)-4,5-dihydro-1H-1,3,6-triaza-benzo[e]azulen-6- |
| | yl]-methanone |
| 40 | (3′-Methoxy-3,4,5,6-tetrahydro-2H-[1,2′]bipyridinyl-4-yl)-[9-methyl-2- |
| | (tetrahydro-pyran-4-yl)-4,5-dihydro-1H-1,3,6-triaza-benzo[e]azulen-6- |
| | yl]-methanone |
| 41 | (4′-Methoxy-3,4,5,6-tetrahydro-2H-[1,2′]bipyridinyl-4-yl)-[9-methyl-2- |
| | (tetrahydro-pyran-4-yl)-4,5-dihydro-1H-1,3,6-triaza-benzo[e]azulen-6- |
| | yl]-methanone |
| 42 | (5′-Methoxy-3,4,5,6-tetrahydro-2H-[1,2′]bipyridinyl-4-yl)-[9-methyl-2- |
| | (tetrahydro-pyran-4-yl)-4,5-dihydro-1H-1,3,6-triaza-benzo[e]azulen-6- |
| | yl]-methanone |
| 43 | (9-Methyl-2-pyrrolidin-1-yl-4,5-dihydro-1H-1,3,6-triaza-benzo[e]azulen- |
| | 6-yl)-(5′-methyl-3,4,5,6-tetrahydro-2H-[1,2′]bipyridinyl-4-yl)-methanone |
| 44 | (9-Chloro-2-methyl-4,5-dihydro-1H-1,3,6-triaza-benzo[e]azulen-6-yl)- |
| | (3,4,5,6-tetrahydro-2H-[1,2′]bipyridinyl-4-yl)-methanone |
| 45 | (9-Chloro-2-methyl-4,5-dihydro-1H-1,3,6-triaza-benzo[e]azulen-6-yl)- |
| | (5′-methyl-3,4,5,6-tetrahydro-2H-[1,2′]bipyridinyl-4-yl)-methanone |
| 46 | (9-Chloro-2-ethyl-4,5-dihydro-1H-1,3,6-triaza-benzo[e]azulen-6-yl)- |
| | (3,4,5,6-tetrahydro-2H-[1,2′]bipyridinyl-4-yl)-methanone |
| 47 | (9-Chloro-2-propyl-4,5-dihydro-1H-1,3,6-triaza-benzo[e]azulen-6-yl)- |
| | (3,4,5,6-tetrahydro-2H-[1,2′]bipyridinyl-4-yl)-methanone |
| 48 | (9-Chloro-2-cyclopropyl-4,5-dihydro-1H-1,3,6-triaza-benzo[e]azulen-6- |
| | yl)-(3,4,5,6-tetrahydro-2H-[1,2′]bipyridinyl-4-yl)-methanone |
| 49 | (9-Chloro-2-cyclohexyl-4,5-dihydro-1H-1,3,6-triaza-benzo[e]azulen-6- |
| | yl)-(3,4,5,6-tetrahydro-2H-[1,2′]bipyridinyl-4-yl)-methanone |
| 50 | (9-Chloro-2-piperidin-1-yl-4,5-dihydro-1H-1,3,6-triaza-benzo[e]azulen- |
| | 6-yl)-(3,4,5,6-tetrahydro-2H-[1,2′]bipyridinyl-4-yl)-methanone |
| 51 | [9-Chloro-2-(4-methyl-piperazin-1-yl)-4,5-dihydro-1H-1,3,6-triaza- |
| | benzo[e]azulen-6-yl]-(3,4,5,6-tetrahydro-2H-[1,2′]bipyridinyl-4-yl)- |
| | methanone |
| 52 | [9-Chloro-2-(4-methyl-piperazin-1-yl)-4,5-dihydro-1H-1,3,6-triaza- |
| | benzo[e]azulen-6-yl]-(5′-methyl-3,4,5,6-tetrahydro-2H-[1,2′]bipyridinyl- |
| | 4-yl)-methanone |
| 53 | (9-Chloro-2-phenyl-4,5-dihydro-1H-1,3,6-triaza-benzo[e]azulen-6-yl)- |
| | (3,4,5,6-tetrahydro-2H-[1,2′]bipyridinyl-4-yl)-methanone |
| 54 | [9-Chloro-2-(2-fluoro-phenyl)-4,5-dihydro-1H-1,3,6-triaza- |
| | benzo[e]azulen-6-yl]-(3,4,5,6-tetrahydro-2H-[1,2′]bipyridinyl-4-yl)- |
| | methanone |
| 55 | [9-Chloro-2-(3-fluoro-phenyl)-4,5-dihydro-1H-1,3,6-triaza- |
| | benzo[e]azulen-6-yl]-(3,4,5,6-tetrahydro-2H-[1,2′]bipyridinyl-4-yl)- |
| | methanone |
| 56 | (2-Benzyl-9-chloro-4,5-dihydro-1H-1,3,6-triaza-benzo[e]azulen-6-yl)- |
| | (3,4,5,6-tetrahydro-2H-[1,2′]bipyridinyl-4-yl)-methanone |
| 57 | (9-Chloro-2-morpholin-4-yl-4,5-dihydro-1H-1,3,6-triaza-benzo[e]azulen- |
| | 6-yl)-(3,4,5,6-tetrahydro-2H-[1,2′]bipyridinyl-4-yl)-methanone |
| 58 | (9-Chloro-2-morpholin-4-yl-4,5-dihydro-1H-1,3,6-triaza-benzo[e]azulen- |
| | 6-yl)-(4′-methyl-3,4,5,6-tetrahydro-2H-[1,2′]bipyridinyl-4-yl)-methanone |
| 59 | (9-Chloro-2-morpholin-4-yl-4,5-dihydro-1H-1,3,6-triaza-benzo[e]azulen- |
| | 6-yl)-(5′-methyl-3,4,5,6-tetrahydro-2H-[1,2′]bipyridinyl-4-yl)-methanone |
| 60 | (6′-Methyl-3,4,5,6-tetrahydro-2H-[1,2′]bipyridinyl-4-yl)-(2-morpholin-4- |
| | yl-4,5-dihydro-3H-1,3,6-triaza-benzo[e]azulen-6-yl)-methanone |
| 61 | (2-Ethyl-9-methyl-4,5-dihydro-1H-1,3,6-triaza-benzo[e]azulen-6-yl)-(6′- |
| | methyl-3,4,5,6-tetrahydro-2H-[1,2′]bipyridinyl-4-yl)-methanone |
| 62 | (9-Methyl-2-propyl-4,5-dihydro-1H-1,3,6-triaza-benzo[e]azulen-6-yl)-(6′- |
| | methyl-3,4,5,6-tetrahydro-2H-[1,2′]bipyridinyl-4-yl)-methanone |
| 63 | (9-Methyl-2-morpholin-4-yl-4,5-dihydro-1H-1,3,6-triaza-benzo[e]azulen- |
| | 6-yl)-(6′-methyl-3,4,5,6-tetrahydro-2H-[1,2′]bipyridinyl-4-yl)-methanone |
| 64 | (6′-Methoxy-3,4,5,6-tetrahydro-2H-[1,2′]bipyridinyl-4-yl)-(9-methyl-2- |
| | morpholin-4-yl-4,5-dihydro-1H-1,3,6-triaza-benzo[e]azulen-6-yl)- |
| | methanone |
| 65 | (6′-Methyl-3,4,5,6-tetrahydro-2H-[1,2′]bipyridinyl-4-yl)-[9-methyl-2- |
| | (tetrahydro-pyran-4-yl)-4,5-dihydro-1H-1,3,6-triaza-benzo[e]azulen-6- |
| | yl]-methanone |
| 66 | (6′-Methoxy-3,4,5,6-tetrahydro-2H-[1,2′]bipyridinyl-4-yl)-[9-methyl-2- |
| | (tetrahydro-pyran-4-yl)-4,5-dihydro-1H-1,3,6-triaza-benzo[e]azulen-6- |
| | yl]-methanone |
| 67 | (9-Chloro-2-morpholin-4-yl-4,5-dihydro-1H-1,3,6-triaza-benzo[e]azulen- |
| | 6-yl)-(6′-methyl-3,4,5,6-tetrahydro-2H-[1,2′]bipyridinyl-4-yl)-methanone |
| 68 | (9-Chloro-2-morpholin-4-yl-4,5-dihydro-1H-1,3,6-triaza-benzo[e]azulen- |
| | 6-yl)-(6′-methoxy-3,4,5,6-tetrahydro-2H-[1,2′]bipyridinyl-4-yl)- |
| | methanone |
| 69 | (9-Methyl-2-morpholin-4-yl-4,5-dihydro-1H-1,3,6-triaza-benzo[e]azulen- |
| | 6-yl)-(4′-methyl-3,4,5,6-tetrahydro-2H-[1,3′]bipyridinyl-4-yl)-methanone |
| 70 | (9-Methyl-2-morpholin-4-yl-4,5-dihydro-1H-1,3,6-triaza-benzo[e]azulen- |
| | 6-yl)-(5′-methyl-3,4,5,6-tetrahydro-2H-[1,3′]bipyridinyl-4-yl)-methanone |
| 71 | (9-Methyl-2-morpholin-4-yl-4,5-dihydro-3H-1,3,6-triaza-benzo[e]azulen- |
| | 6-yl)-(2′-methyl-3,4,5,6-tetrahydro-2H-[1,3′]bipyridinyl-4-yl)-methanone |
| 72 | (9-Chloro-2-methyl-4,5-dihydro-1H-1,3,6-triaza-benzo[e]azulen-6-yl)-(1- |
| | phenyl-piperidin-4-yl)-methanone |
| 73 | (9-Chloro-2-ethyl-4,5-dihydro-1H-1,3,6-triaza-benzo[e]azulen-6-yl)-(1- |
| | phenyl-piperidin-4-yl)-methanone |
| 74 | (9-Chloro-2-propyl-4,5-dihydro-1H-1,3,6-triaza-benzo[e]azulen-6-yl)-(1- |
| | phenyl-piperidin-4-yl)-methanone |
| 75 | (9-Chloro-2-cyclopropyl-4,5-dihydro-1H-1,3,6-triaza-benzo[e]azulen-6- |
| | yl)-(1-phenyl-piperidin-4-yl)-methanone |
| 76 | (9-Chloro-2-cyclohexyl-4,5-dihydro-1H-1,3,6-triaza-benzo[e]azulen-6- |
| | yl)-(1-phenyl-piperidin-4-yl)-methanone |
| 77 | (9-Chloro-2-piperidin-1-yl-4,5-dihydro-1H-1,3,6-triaza-benzo[e]azulen- |
| | 6-yl)-(1-phenyl-piperidin-4-yl)-methanone |
| 78 | [9-Chloro-2-(4-methyl-piperazin-1-yl)-4,5-dihydro-1H-1,3,6-triaza- |
| | benzo[e]azulen-6-yl]-(1-phenyl-piperidin-4-yl)-methanone |
| 79 | (9-Chloro-2-phenyl-4,5-dihydro-1H-1,3,6-triaza-benzo[e]azulen-6-yl)-(1- |
| | phenyl-piperidin-4-yl)-methanone |
| 80 | [9-Chloro-2-(2-fluoro-phenyl)-4,5-dihydro-1H-1,3,6-triaza- |
| | benzo[e]azulen-6-yl]-(1-phenyl-piperidin-4-yl)-methanone |
| 81 | [9-Chloro-2-(3-fluoro-phenyl)-4,5-dihydro-1H-1,3,6-triaza- |
| | benzo[e]azulen-6-yl]-(1-phenyl-piperidin-4-yl)-methanone |
| 82 | (2-Benzyl-9-chloro-4,5-dihydro-1H-1,3,6-triaza-benzo[e]azulen-6-yl)-(1- |
| | phenyl-piperidin-4-yl)-methanone |
| 83 | (9-Chloro-2-methyl-4,5-dihydro-1H-1,3,6-triaza-benzo[e]azulen-6-yl)-[1- |
| | (2-fluoro-phenyl)-piperidin-4-yl]-methanone |
| 84 | (9-Chloro-2-methyl-4,5-dihydro-1H-1,3,6-triaza-benzo[e]azulen-6-yl)-[1- |
| | (3-fluoro-phenyl)-piperidin-4-yl]-methanone |
| 85 | (9-Chloro-2-methyl-4,5-dihydro-1H-1,3,6-triaza-benzo[e]azulen-6-yl)-[1- |
| | (4-fluoro-phenyl)-piperidin-4-yl]-methanone |
| 86 | (9-Chloro-2-methyl-4,5-dihydro-1H-1,3,6-triaza-benzo[e]azulen-6-yl)-[1- |
| | (2-chloro-phenyl)-piperidin-4-yl]-methanone |
| 87 | (9-Chloro-2-methyl-4,5-dihydro-1H-1,3,6-triaza-benzo[e]azulen-6-yl)-[1- |
| | (3-chloro-phenyl)-piperidin-4-yl]-methanone |
| 88 | (9-Chloro-2-methyl-4,5-dihydro-1H-1,3,6-triaza-benzo[e]azulen-6-yl)-[1- |
| | (4-chloro-phenyl)-piperidin-4-yl]-methanone |
| 89 | (9-Chloro-2-methyl-4,5-dihydro-1H-1,3,6-triaza-benzo[e]azulen-6-yl)-(1- |
| | o-tolyl-piperidin-4-yl)-methanone |
| 90 | (9-Chloro-2-methyl-4,5-dihydro-1H-1,3,6-triaza-benzo[e]azulen-6-yl)-(1- |
| | m-tolyl-piperidin-4-yl)-methanone |
| 91 | (9-Chloro-2-methyl-4,5-dihydro-1H-1,3,6-triaza-benzo[e]azulen-6-yl)-(1- |
| | p-tolyl-piperidin-4-yl)-methanone |
| 92 | (9-Chloro-2-methyl-4,5-dihydro-1H-1,3,6-triaza-benzo[e]azulen-6-yl)-[1- |
| | (2-methoxy-phenyl)-piperidin-4-yl]-methanone |
| 93 | (9-Chloro-2-methyl-4,5-dihydro-1H-1,3,6-triaza-benzo[e]azulen-6-yl)-[1- |
| | (3-methoxy-phenyl)-piperidin-4-yl]-methanone |
| 94 | (9-Chloro-2-methyl-4,5-dihydro-1H-1,3,6-triaza-benzo[e]azulen-6-yl)-[1- |
| | (4-methoxy-phenyl)-piperidin-4-yl]-methanone |
| 95 | (9-Chloro-2-methyl-4,5-dihydro-1H-1,3,6-triaza-benzo[e]azulen-6-yl)-[1- |
| | (4-ethyl-phenyl)-piperidin-4-yl]-methanone |
| 96 | (9-Chloro-2-methyl-4,5-dihydro-1H-1,3,6-triaza-benzo[e]azulen-6-yl)-[1- |
| | (4-propyl-phenyl)-piperidin-4-yl]-methanone |
| 97 | (9-Chloro-2-methyl-4,5-dihydro-1H-1,3,6-triaza-benzo[e]azulen-6-yl)-[1- |
| | (4-isopropyl-phenyl)-piperidin-4-yl]-methanone |
| 98 | (3-Methyl-4,10-dihydro-3H-2,3,4,9-tetraaza-benzo[f]azulen-9-yl)-(1- |
| | pyrimidin-2-yl-piperidin-4-yl)-methanone |
| 99 | (3-Cyclohexyl-4,10-dihydro-3H-2,3,4,9-tetraaza-benzo[f]azulen-9-yl)-[1- |
| | (4-trifluoromethyl-pyrimidin-2-yl)-piperidin-4-yl]-methanone |
| 100 | (6-Fluoro-3-methyl-4,10-dihydro-3H-2,3,4,9-tetraaza-benzo[f]azulen-9- |
| | yl)-(1-pyrimidin-2-yl-piperidin-4-yl)-methanone |
| 101 | (3-Ethyl-6-fluoro-4,10-dihydro-3H-2,3,4,9-tetraaza-benzo[f]azulen-9-yl)- |
| | [1-(4-trifluoromethyl-pyrimidin-2-yl)-piperidin-4-yl]-methanone |
| 102 | (3,6-Dimethyl-4,10-dihydro-3H-2,3,4,9-tetraaza-benzo[f]azulen-9-yl)-(1- |
| | pyrimidin-2-yl-piperidin-4-yl)-methanone |
| 103 | (6-Chloro-3-methyl-4,10-dihydro-3H-2,3,4,9-tetraaza-benzo[f]azulen-9- |
| | yl)-(1-pyrimidin-2-yl-piperidin-4-yl)-methanone |
| 104 | (6-Chloro-3-phenyl-4,10-dihydro-3H-2,3,4,9-tetraaza-benzo[f]azulen-9- |
| | yl)-(1-pyrimidin-2-yl-piperidin-4-yl)-methanone |
| 105 | (6-Chloro-3-phenyl-4,10-dihydro-3H-2,3,4,9-tetraaza-benzo[f]azulen-9- |
| | yl)-[1-(5-ethyl-pyrimidin-2-yl)-piperidin-4-yl]-methanone |
| 106 | (3-Methyl-4,10-dihydro-3H-2,3,4,9-tetraaza-benzo[f]azulen-9-yl)- |
| | (3,4,5,6-tetrahydro-2H-[1,2′]bipyridinyl-4-yl)-methanone |
| 107 | (3-Ethyl-6-fluoro-4,10-dihydro-3H-2,3,4,9-tetraaza-benzo[f]azulen-9-yl)- |
| | (5′-methyl-3,4,5,6-tetrahydro-2H-[1,2′]bipyridinyl-4-yl)-methanone |
| 108 | (3-tert-Butyl-6-fluoro-4,10-dihydro-3H-2,3,4,9-tetraaza-benzo[f]azulen- |
| | 9-yl)-(5′-methyl-3,4,5,6-tetrahydro-2H-[1,2′]bipyridinyl-4-yl)-methanone |
| 109 | (3,6-Dimethyl-4,10-dihydro-3H-2,3,4,9-tetraaza-benzo[f]azulen-9-yl)- |
| | (3,4,5,6-tetrahydro-2H-[1,2′]bipyridinyl-4-yl)-methanone |
| 110 | (3,6-Dimethyl-4,10-dihydro-3H-2,3,4,9-tetraaza-benzo[f]azulen-9-yl)-(3′- |
| | methyl-3,4,5,6-tetrahydro-2H-[1,2′]bipyridinyl-4-yl)-methanone |
| 111 | (3,6-Dimethyl-4,10-dihydro-3H-2,3,4,9-tetraaza-benzo[f]azulen-9-yl)-(4′- |
| | methyl-3,4,5,6-tetrahydro-2H-[1,2′]bipyridinyl-4-yl)-methanone |
| 112 | (3,6-Dimethyl-4,10-dihydro-3H-2,3,4,9-tetraaza-benzo[f]azulen-9-yl)-(5′- |
| | methyl-3,4,5,6-tetrahydro-2H-[1,2′]bipyridinyl-4-yl)-methanone |
| 113 | (3,6-Dimethyl-4,10-dihydro-3H-2,3,4,9-tetraaza-benzo[f]azulen-9-yl)-(3′- |
| | methoxy-3,4,5,6-tetrahydro-2H-[1,2′]bipyridinyl-4-yl)-methanone |
| 114 | (3-Benzyl-6-methyl-4,10-dihydro-3H-2,3,4,9-tetraaza-benzo[f]azulen-9- |
| | yl)-(4′-methyl-3,4,5,6-tetrahydro-2H-[1,2′]bipyridinyl-4-yl)-methanone |
| 115 | (6-Chloro-3-methyl-4,10-dihydro-3H-2,3,4,9-tetraaza-benzo[f]azulen-9- |
| | yl)-(3,4,5,6-tetrahydro-2H-[1,2′]bipyridinyl-4-yl)-methanone |
| 116 | (6-Chloro-3-methyl-4,10-dihydro-3H-2,3,4,9-tetraaza-benzo[f]azulen-9- |
| | yl)-(3′-methyl-3,4,5,6-tetrahydro-2H-[1,2′]bipyridinyl-4-yl)-methanone |
| 117 | (6-Chloro-3-methyl-4,10-dihydro-3H-2,3,4,9-tetraaza-benzo[f]azulen-9- |
| | yl)-(4′-methyl-3,4,5,6-tetrahydro-2H-[1,2′]bipyridinyl-4-yl)-methanone |
| 118 | (6-Chloro-3-methyl-4,10-dihydro-3H-2,3,4,9-tetraaza-benzo[f]azulen-9- |
| | yl)-(5′-methyl-3,4,5,6-tetrahydro-2H-[1,2′]bipyridinyl-4-yl)-methanone |
| 119 | (6-Chloro-3-methyl-4,10-dihydro-3H-2,3,4,9-tetraaza-benzo[f]azulen-9- |
| | yl)-(3′-methoxy-3,4,5,6-tetrahydro-2H-[1,2′]bipyridinyl-4-yl)-methanone |
| 120 | (6-Chloro-3-ethyl-4,10-dihydro-3H-2,3,4,9-tetraaza-benzo[f]azulen-9-yl)- |
| | (3′-methyl-3,4,5,6-tetrahydro-2H-[1,2′]bipyridinyl-4-yl)-methanone |
| 121 | (6-Chloro-3-ethyl-4,10-dihydro-3H-2,3,4,9-tetraaza-benzo[f]azulen-9-yl)- |
| | (4′-methyl-3,4,5,6-tetrahydro-2H-[1,2′]bipyridinyl-4-yl)-methanone |
| 122 | (6-Chloro-3-ethyl-4,10-dihydro-3H-2,3,4,9-tetraaza-benzo[f]azulen-9-yl)- |
| | (5′-methyl-3,4,5,6-tetrahydro-2H-[1,2′]bipyridinyl-4-yl)-methanone |
| 123 | (6-Chloro-3-phenyl-4,10-dihydro-3H-2,3,4,9-tetraaza-benzo[f]azulen-9- |
| | yl)-(5′-methyl-3,4,5,6-tetrahydro-2H-[1,2′]bipyridinyl-4-yl)-methanone |
| 124 | (3,6-Dimethyl-4,10-dihydro-3H-2,3,4,9-tetraaza-benzo[f]azulen-9-yl)-(6′- |
| | methyl-3,4,5,6-tetrahydro-2H-[1,2′]bipyridinyl-4-yl)-methanone |
| 125 | (6-Chloro-3-methyl-4,10-dihydro-3H-2,3,4,9-tetraaza-benzo[f]azulen-9- |
| | yl)-(6′-methyl-3,4,5,6-tetrahydro-2H-[1,2′]bipyridinyl-4-yl)-methanone |
| 126 | (6-Chloro-3-ethyl-4,10-dihydro-3H-2,3,4,9-tetraaza-benzo[f]azulen-9-yl)- |
| | (6′-methyl-3,4,5,6-tetrahydro-2H-[1,2′]bipyridinyl-4-yl)-methanone |
| 127 | (3,6-Dimethyl-4,10-dihydro-3H-2,3,4,9-tetraaza-benzo[f]azulen-9-yl)-[1- |
| | (2-fluoro-phenyl)-piperidin-4-yl]-methanone |
| 128 | (3,6-Dimethyl-4,10-dihydro-3H-2,3,4,9-tetraaza-benzo[f]azulen-9-yl)-(1- |
| | m-tolyl-piperidin-4-yl)-methanone |
| 129 | (3,6-Dimethyl-4,10-dihydro-3H-2,3,4,9-tetraaza-benzo[f]azulen-9-yl)-(1- |
| | p-tolyl-piperidin-4-yl)-methanone |
| 130 | (3,6-Dimethyl-4,10-dihydro-3H-2,3,4,9-tetraaza-benzo[f]azulen-9-yl)-[1- |
| | (3-methoxy-phenyl)-piperidin-4-yl]-methanone |
| 131 | (3,6-Dimethyl-4,10-dihydro-3H-2,3,4,9-tetraaza-benzo[f]azulen-9-yl)-[1- |
| | (4-methoxy-phenyl)-piperidin-4-yl]-methanone |
| 132 | (3,6-Dimethyl-4,10-dihydro-3H-2,3,4,9-tetraaza-benzo[f]azulen-9-yl)-[1- |
| | (4-ethyl-phenyl)-piperidin-4-yl]-methanone |
| 133 | (3,6-Dimethyl-4,10-dihydro-3H-2,3,4,9-tetraaza-benzo[f]azulen-9-yl)-[1- |
| | (4-trifluoromethyl-phenyl)-piperidin-4-yl]-methanone |
| 134 | (6-Chloro-3-methyl-4,10-dihydro-3H-2,3,4,9-tetraaza-benzo[f]azulen-9- |
| | yl)-[1-(2-fluoro-phenyl)-piperidin-4-yl]-methanone |
| 135 | (6-Chloro-3-methyl-4,10-dihydro-3H-2,3,4,9-tetraaza-benzo[f]azulen-9- |
| | yl)-(1-m-tolyl-piperidin-4-yl)-methanone |
| 136 | (6-Chloro-3-methyl-4,10-dihydro-3H-2,3,4,9-tetraaza-benzo[f]azulen-9- |
| | yl)-(1-p-tolyl-piperidin-4-yl)-methanone |
| 137 | (6-Chloro-3-methyl-4,10-dihydro-3H-2,3,4,9-tetraaza-benzo[f]azulen-9- |
| | yl)-[1-(3-methoxy-phenyl)-piperidin-4-yl]-methanone |
| 138 | (6-Chloro-3-methyl-4,10-dihydro-3H-2,3,4,9-tetraaza-benzo[f]azulen-9- |
| | yl)-[1-(4-methoxy-phenyl)-piperidin-4-yl]-methanone |
| 139 | (6-Chloro-3-methyl-4,10-dihydro-3H-2,3,4,9-tetraaza-benzo[f]azulen-9- |
| | yl)-[1-(4-ethyl-phenyl)-piperidin-4-yl]-methanone |
| 140 | (6-Chloro-3-methyl-4,10-dihydro-3H-2,3,4,9-tetraaza-benzo[f]azulen-9- |
| | yl)-[1-(4-trifluoromethyl-phenyl)-piperidin-4-yl]-methanone |
| 141 | (3,6-Dimethyl-4,10-dihydro-3H-2,3,4,9-tetraaza-benzo[f]azulen-9-yl)-(6′- |
| | methyl-3,4,5,6-tetrahydro-2H-[1,3′]bipyridinyl-4-yl)-methanone |
| 142 | (6-Chloro-3-methyl-4,10-dihydro-3H-2,3,4,9-tetraaza-benzo[f]azulen-9- |
| | yl)-(6′-methyl-3,4,5,6-tetrahydro-2H-[1,3′]bipyridinyl-4-yl)-methanone |
| 143 | (5H,11H-Benzo[e]pyrrolo[1,2-a][1,4]diazepin-10-yl)-(1-pyrimidin-2-yl- |
| | piperidin-4-yl)-methanone |
| 144 | (5H,11H-Benzo[e]pyrrolo[1,2-a][1,4]diazepin-10-yl)-(3,4,5,6-tetrahydro- |
| | 2H-[1,2′]bipyridinyl-4-yl)-methanone |
| 145 | (5H,11H-Benzo[e]pyrrolo[1,2-a][1,4]diazepin-10-yl)-(5′-methyl-3,4,5,6- |
| | tetrahydro-2H-[1,2′]bipyridinyl-4-yl)-methanone |
| 146 | (5H,11H-Benzo[e]pyrrolo[1,2-a][1,4]diazepin-10-yl)-(3,4,5,6-tetrahydro- |
| | 2H-[1,3′]bipyridinyl-4-yl)-methanone |
| 147 | (5H,11H-Benzo[e]pyrrolo[1,2-a][1,4]diazepin-10-yl)-(3,4,5,6-tetrahydro- |
| | 2H-[1,4′]bipyridinyl-4-yl)-methanone |
| 148 | (5H,11H-Benzo[e]pyrrolo[1,2-a][1,4]diazepin-10-yl)-(1-pyrazin-2-yl- |
| | piperidin-4-yl)-methanone |
| 149 | (5H,11H-Benzo[e]pyrrolo[1,2-a][1,4]diazepin-10-yl)-(1-phenyl- |
| | piperidin-4-yl)-methanone |
| 150 | (7-Fluoro-5H,11H-benzo[e]pyrrolo[1,2-a][1,4]diazepin-10-yl)-(3,4,5,6- |
| | tetrahydro-2H-[1,2′]bipyridinyl-4-yl)-methanone |
| 151 | (7-Fluoro-5H,11H-benzo[e]pyrrolo[1,2-a][1,4]diazepin-10-yl)-(1-phenyl- |
| | piperidin-4-yl)-methanone |
| 152 | (7-Methyl-5H,11H-benzo[e]pyrrolo[1,2-a][1,4]diazepin-10-yl)-(3,4,5,6- |
| | tetrahydro-2H-[1,2′]bipyridinyl-4-yl)-methanone |
| 153 | (7-Methyl-5H,11H-benzo[e]pyrrolo[1,2-a][1,4]diazepin-10-yl)-(1-phenyl- |
| | piperidin-4-yl)-methanone |
| 154 | (5H,11H-Benzo[e]pyrrolo[1,2-a][1,4]diazepin-10-yl)-[1-(2-fluoro- |
| | phenyl)-piperidin-4-yl]-methanone |
| 155 | (5H,11H-Benzo[e]pyrrolo[1,2-a][1,4]diazepin-10-yl)-[1-(3-fluoro- |
| | phenyl)-piperidin-4-yl]-methanone |
| 156 | (5H,11H-Benzo[e]pyrrolo[1,2-a][1,4]diazepin-10-yl)-[1-(4-fluoro- |
| | phenyl)-piperidin-4-yl]-methanone |
| 157 | (5H,11H-Benzo[e]pyrrolo[1,2-a][1,4]diazepin-10-yl)-[1-(2-chloro- |
| | phenyl)-piperidin-4-yl]-methanone |
| 158 | (5H,11H-Benzo[e]pyrrolo[1,2-a][1,4]diazepin-10-yl)-[1-(4-chloro- |
| | phenyl)-piperidin-4-yl]-methanone |
| 159 | (5H,11H-Benzo[e]pyrrolo[1,2-a][1,4]diazepin-10-yl)-(1-o-tolyl-piperidin- |
| | 4-yl)-methanone |
| 160 | (5H,11H-Benzo[e]pyrrolo[1,2-a][1,4]diazepin-10-yl)-(1-m-tolyl- |
| | piperidin-4-yl)-methanone |
| 161 | (5H,11H-Benzo[e]pyrrolo[1,2-a][1,4]diazepin-10-yl)-(1-p-tolyl-piperidin- |
| | 4-yl)-methanone |
| 162 | (5H,11H-Benzo[e]pyrrolo[1,2-a][1,4]diazepin-10-yl)-[1-(2-methoxy- |
| | phenyl)-piperidin-4-yl]-methanone |
| 163 | (5H,11H-Benzo[e]pyrrolo[1,2-a][1,4]diazepin-10-yl)-[1-(3-methoxy- |
| | phenyl)-piperidin-4-yl]-methanone |
| 164 | (5H,11H-Benzo[e]pyrrolo[1,2-a][1,4]diazepin-10-yl)-[1-(4-methoxy- |
| | phenyl)-piperidin-4-yl]-methanone |
| 165 | (5H,11H-Benzo[e]pyrrolo[1,2-a][1,4]diazepin-10-yl)-[1-(4-ethyl-phenyl)- |
| | piperidin-4-yl]-methanone |
| 166 | (5H,11H-Benzo[e]pyrrolo[1,2-a][1,4]diazepin-10-yl)-[1-(4-propyl- |
| | phenyl)-piperidin-4-yl]-methanone |
| 167 | (5H,11H-Benzo[e]pyrrolo[1,2-a][1,4]diazepin-10-yl)-[1-(4-isopropyl- |
| | phenyl)-piperidin-4-yl]-methanone |
| 168 | (5H,11H-Benzo[e]pyrrolo[1,2-a][1,4]diazepin-10-yl)-(1-biphenyl-3-yl- |
| | piperidin-4-yl)-methanone |
| 169 | (5H,11H-Benzo[e]pyrrolo[1,2-a][1,4]diazepin-10-yl)-[1-(4- |
| | trifluoromethyl-phenyl)-piperidin-4-yl]-methanone |
| 170 | 3-[4-(5H,11H-Benzo[e]pyrrolo[1,2-a][1,4]diazepine-10-carbonyl)- |
| | piperidin-1-yl]-benzonitrile |
| 171 | 4-[4-(5H,11H-Benzo[e]pyrrolo[1,2-a][1,4]diazepine-10-carbonyl)- |
| | piperidin-1-yl]-benzonitrile |
| 172 | (5H,11H-Benzo[e]pyrrolo[1,2-a][1,4]diazepin-10-yl)-[1-(2-nitro-phenyl)- |
| | piperidin-4-yl]-methanone |
| 173 | (5H,11H-Benzo[e]pyrrolo[1,2-a][1,4]diazepin-10-yl)-[1-(3-nitro-phenyl)- |
| | piperidin-4-yl]-methanone |
| 174 | (5H,11H-Benzo[e]pyrrolo[1,2-a][1,4]diazepin-10-yl)-[1-(4-nitro-phenyl)- |
| | piperidin-4-yl]-methanone |
| 176 | [1-(4-Amino-phenyl)-piperidin-4-yl]-(5H,11H-benzo[e]pyrrolo[1,2- |
| | a][1,4]diazepin-10-yl)-methanone |
| 178 | [1-(4-Aminomethyl-phenyl)-piperidin-4-yl]-(5H,11H- |
| | benzo[e]pyrrolo[1,2-a][1,4]diazepin-10-yl)-methanone |

### Biological Methods

The primary assay which may be used to determine the ability of the compounds of formula (1) to inhibit the vasopressin V₁ₐ receptor is an in vitro functional calcium mobilisation assay (FLIPR) that measures antagonist activity at a cloned human V₁ₐ receptor. The assay is described below.

The antagonist activity of compounds of formula (1) were determined in a Calcium (Ca2+) mobilisation assay using whole cells (human brain astrocytoma 1321N1 cells, ex Perkin Elmer) genetically modified to stably express a cloned human V₁ₐ receptor. Dose response curves were determined by displacement of a single concentration of agonist (250 pM AVP, ex Sigma) with increasing concentrations of compound. A pIC50 value is determined by non-linear regression to a 4-parameter logistic equation and a functional pKi (fpKi) derived using Equation 1.

$$Modification of Cheng \text{-} Prusoff to derive fpKi \\ Functional pKi = Log [ \frac{{IC}_{50}}{[ \frac{A}{{A}_{50}} ] + 1} ] \qquad Equation 1$$

where A=agonist single conc, and A50=the agonist EC₅₀.

Data acquired from this assays are shown in the table below:

TABLE 19
(In vitro activity)

| Example | Mean fpKi (Human V1a) |
|---|---|
| 1 | 9.01 |
| 2 | 8.84 |
| 3 | 9.01 |
| 4 | 8.83 |
| 5 | 8.75 |
| 6 | 6.04 |
| 7 | 7.31 |
| 8 | 6.82 |
| 9 | 7.74 |
| 10 | 8.30 |
| 11 | 8.43 |
| 12 | 8.67 |
| 13 | 8.10 |
| 14 | 8.60 |
| 15 | 8.71 |
| 16 | 8.53 |
| 17 | 8.30 |
| 18 | 8.64 |
| 19 | 8.63 |
| 20 | 7.91 |
| 21 | 8.35 |
| 22 | 8.69 |
| 23 | 8.67 |
| 24 | 8.06 |
| 25 | 8.82 |
| 26 | 7.74 |
| 27 | 8.35 |
| 28 | 7.87 |
| 29 | 8.57 |
| 30 | 8.75 |
| 31 | 8.42 |
| 32 | 8.80 |
| 33 | 9.01 |
| 34 | 8.57 |
| 35 | 8.15 |
| 36 | 7.01 |
| 37 | 8.36 |
| 38 | 8.65 |
| 39 | 8.70 |
| 40 | 8.90 |
| 41 | 8.63 |
| 42 | 8.25 |
| 43 | 8.12 |
| 44 | 7.90 |
| 45 | 8.20 |
| 46 | 7.87 |
| 47 | 7.93 |
| 48 | 7.99 |
| 49 | 7.63 |
| 50 | 7.61 |
| 51 | 7.76 |
| 52 | 8.96 |
| 53 | 7.19 |
| 54 | 7.12 |
| 55 | 7.01 |
| 56 | 7.10 |
| 57 | 8.60 |
| 58 | 9.19 |
| 59 | 9.10 |
| 60 | 7.89 |
| 61 | 8.13 |
| 62 | 8.13 |
| 63 | 8.73 |
| 64 | 8.20 |
| 65 | 8.12 |
| 66 | 7.51 |
| 67 | 8.87 |
| 68 | 8.72 |
| 69 | 7.86 |
| 70 | 8.18 |
| 71 | 7.78 |
| 72 | 8.04 |
| 73 | 8.21 |
| 74 | 7.82 |
| 75 | 7.96 |
| 76 | 7.55 |
| 77 | 7.74 |
| 78 | 8.06 |
| 79 | 7.11 |
| 80 | 7.09 |
| 81 | 7.03 |
| 82 | 7.37 |
| 83 | 7.58 |
| 84 | 7.42 |
| 85 | 7.96 |
| 86 | 7.50 |
| 87 | 7.16 |
| 88 | 8.21 |
| 89 | 7.59 |
| 90 | 8.02 |
| 91 | 8.90 |
| 92 | 7.64 |
| 93 | 8.60 |
| 94 | 7.79 |
| 95 | 8.50 |
| 96 | 7.87 |
| 97 | 7.65 |
| 98 | 6.85 |
| 99 | 7.45 |
| 100 | 6.84 |
| 101 | 6.24 |
| 102 | 6.82 |
| 103 | 7.12 |
| 104 | 7.85 |
| 105 | 7.86 |
| 106 | 7.90 |
| 107 | 8.72 |
| 108 | 8.27 |
| 109 | 7.89 |
| 110 | 8.34 |
| 111 | 8.62 |
| 112 | 8.55 |
| 113 | 7.52 |
| 114 | 7.70 |
| 115 | 8.16 |
| 116 | 8.57 |
| 117 | 8.70 |
| 118 | 8.76 |
| 119 | 7.72 |
| 120 | 9.04 |
| 121 | 9.37 |
| 122 | 9.22 |
| 123 | 8.70 |
| 124 | 8.02 |
| 125 | 8.23 |
| 126 | 8.92 |
| 127 | 8.70 |
| 128 | 8.88 |
| 129 | 9.49 |
| 230 | 8.61 |
| 131 | 8.51 |
| 132 | 9.13 |
| 133 | 8.27 |
| 134 | 8.76 |
| 135 | 8.89 |
| 136 | 9.21 |
| 137 | 8.75 |
| 138 | 8.58 |
| 139 | 9.04 |
| 140 | 8.07 |
| 141 | 8.29 |
| 142 | 8.54 |
| 143 | 6.93 |
| 144 | 8.50 |
| 145 | 7.82 |
| 146 | 7.50 |
| 147 | 5.83 |
| 148 | 7.55 |
| 149 | 9.23 |
| 150 | 8.72 |
| 151 | 9.10 |
| 152 | 9.20 |
| 153 | 9.63 |
| 154 | 8.83 |
| 155 | 8.34 |
| 156 | 8.84 |
| 157 | 8.12 |
| 158 | 8.32 |
| 159 | 7.98 |
| 160 | 8.50 |
| 161 | 8.95 |
| 162 | 8.50 |
| 163 | 8.25 |
| 164 | 7.85 |
| 165 | 8.81 |
| 166 | 8.41 |
| 167 | 8.44 |
| 168 | 6.82 |
| 169 | 7.51 |
| 170 | 7.59 |
| 171 | 6.67 |
| 172 | 7.35 |
| 173 | 7.77 |
| 174 | 6.79 |
| 175 | 8.40 |
| 176 | 8.60 |
| 177 | 7.54 |
| 178 | 7.01 |

1. A compound of formula (1): 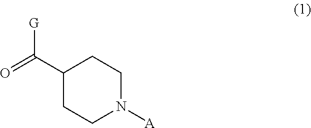
wherein,
G is a fused azepine selected from formula (2), (3), or (4), 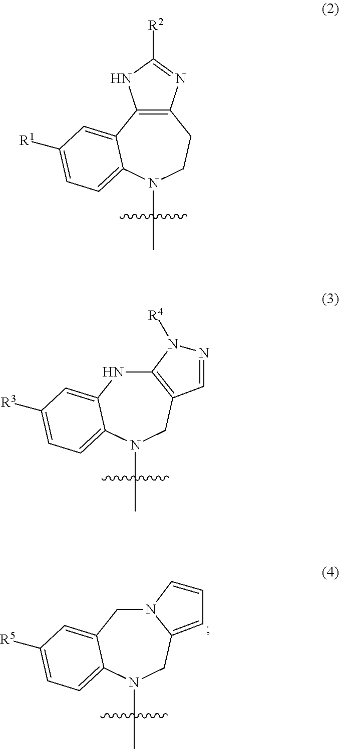
wherein,
R¹ is H, halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-;
R² is (C₁-C₁₀)alkyl, (C₁-C₆)alkoxy, (C₂-C₆)alkenyl, (C₃-C₁₀)cyclo alkyl, heterocycloalkyl, aryl, heteroaryl, aryl(C₁-C₄)alkyl- or heteroaryl(C₁-C₄)alkyl-;
R³ is H, halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-;
R⁴ is (C₁-C₁₀)alkyl, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-;
R⁵ is H, halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- or heteroaryl(C₁-C₄)alkyl-; and
A is phenyl or a 5- or 6-membered aromatic ring containing 1, 2 or 3 N atoms, wherein said phenyl or said 5- or 6-membered ring are optionally substituted with 1 to 3 substituents independently chosen from the group consisting of halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl-, heteroaryl(C₁-C₄)alkyl-, CF₃, CN, NO₂, OH, CO₂R^{d} and NR^{d}R^{e},
wherein
each alkyl may independently be optionally substituted with 1 or 2 substituents independently selected from (C₃-C₁₀)cycloalkyl, (C₁-C₆)alkoxy, OH, CN, CF₃, CO₂R^{p}, halo and NR^{p}R^{q};
each alkenyl may independently be optionally substituted with 1 or 2 substituents independently selected from (C₃-C₁₀)cycloalkyl, (C₁-C₆)alkoxy, OH, CN, CF₃, CO₂R^{r}, halo and N^{r}R^{s};
each alkoxy may independently be optionally be substituted with 1 or 2 substituents independently selected from (C₃-C₁₀)cycloalkyl, OH, CN, CF₃, CO₂R^{t}, halo and NR^{t}R^{u};
each cycloalkyl is independently a non-aromatic mono- or bicylic hydrocarbon ring, optionally fused to an aryl group, wherein said cycloalkyl ring may optionally contain up to 2 double bonds; and wherein, unless otherwise stated, said cycloalkyl may optionally be substituted with 1 or 2 substituents independently selected from (C₁-C₆)alkyl, (C₁-C₆)alkoxy, OH, CN, CF₃, CO₂R^{v}, halo and NR^{v}R^{w};
each heterocycloalkyl is independently a C-linked or N-linked non-aromatic, 3-10 membered mono- or bicyclic ring, wherein said heterocycloalkyl ring may contain 1, 2 or 3 ring members independently selected from N, NR^{x}, S(O)_{y} and O; and said heterocycloalkyl ring may optionally contain 1 or 2 double bonds, and is optionally substituted on carbon with 1 or 2 substituents independently selected from (C₁-C₆)alkyl, (C₁-C₆)alkoxy, OH, CN, CF₃, halo, CO₂R^{x}, NR^{x}R^{y} and aryl;
aryl is a 6 or 10 membered aromatic mono- or bicyclic ring system; wherein, unless otherwise stated, each occurrence of aryl may be optionally substituted with up to 5 substituents independently selected from (C₁-C₆)alkyl, (C₁-C₆)alkoxy, OH, halo, CN, CO₂R^{a}, CF₃ and NR^{a}R^{z};
heteroaryl is a 5, 6, 9 or 10 membered aromatic mono- or bicyclic ring system, containing 1 or 2 N atoms and, optionally, an NR^{b} ring member, or one NR^{b} ring member and an S or an O atom, or one S atom, or one O atom; wherein, unless otherwise stated, said heteroaryl may be optionally substituted with 1, 2 or 3 substituents independently selected from (C₁-C₆)alkyl, (C₁-C₆)alkoxy, OH, halo, CN, CO₂R^{b}, CF₃ and NR^{b}R^{c}; wherein
R^{p}, R^{q}, R^{r}, R^{s}, R^{t}, R^{u}, R^{v}, R^{w}, R^{x}, R^{y}, R^{a}, R^{z}, R^{b}, R^{c}, R^{d} and R^{e} are each independently selected from H and (C₁-C₆)alkyl; and
y is 0, 1 or 2;
or a pharmaceutically acceptable salt or solvate thereof.

2. A compound according to claim 1, wherein A is a phenyl or a 6-membered aromatic ring containing 1, 2 or 3 N atoms and wherein said phenyl or said 6-membered ring are optionally substituted with 1 to 3 substituents independently chosen from the group consisting of halo, (C₁-C₁₀)alkyl, (C₁-C₆)alkoxy, (C₂-C₆)alkenyl, (C₃-C₁₀)cyclo alkyl, heterocycloalkyl, aryl, heteroaryl, aryl(C₁-C₄)alkyl-, heteroaryl(C₁-C₄)alkyl-, CF₃, CN, NO₂, OH, CO₂R^{d} and NR^{d}R^{e}, wherein R^{d} and R^{e} are as previously defined.

3. A compound according to claim 1, wherein A is selected from the group consisting of: 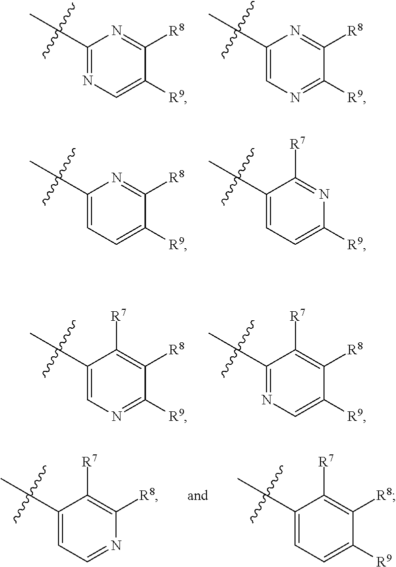
wherein each R⁷, R⁸ and R⁹ is independently selected from the group consisting of
H, halo, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl-, heteroaryl(C₁-C₄)alkyl-, CF₃, CN, NO₂, OH, CO₂R^{d} and NR^{d}R^{e}, wherein R^{d} and R^{e} are each independently selected from H and (C₁-C₆)alkyl.

4. A compound according to claim 3, wherein at least one of R⁷-R⁹ is H.

5. A compound according to claim 1, wherein G is a fused azepine of general formula 2.

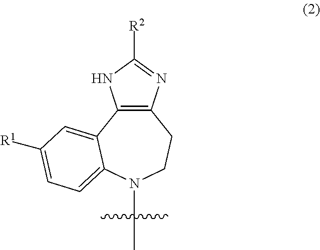

6. A compound according to claim 5, wherein R¹ is H, halo or (C₁-C₆)alkyl.

7. A compound according to claim 5 or claim 6, wherein R² is (C₁-C₆)alkyl, aryl, heterocycloalkyl or aryl(C₁-C₄)alkyl-.

8. A compound according to claim 1, wherein G is a fused azepine of general formula 3.

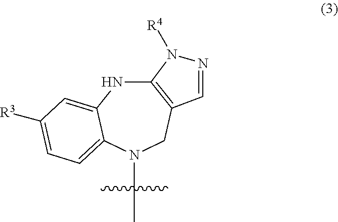

9. A compound according to claim 8, wherein R³ is H, halo or (C₁-C₆)alkyl.

10. A compound according to claim 8 or claim 9, wherein R⁴ is (C₁-C₆)alkyl, aryl or aryl(C₁-C₄)alkyl-.

11. A compound according to claim 1, wherein G is a fused azepine of general formula 4.

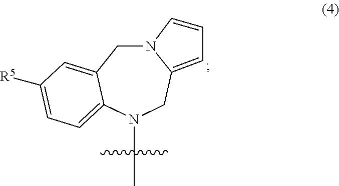

12. A compound according to claim 11, wherein R⁵ is H, halo or (C₁-C₆)alkyl.

13. A compound according to claim 1, selected from the group consisting of:
(9-Methyl-2-morpholin-4-yl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(3′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)methanone;
(9-Fluoro-2-morpholin-4-yl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(6′-methyl-3,4,5,6-tetrahydro-2H-[1,3′]bipyridinyl-4-yl)-methanone;
(2-Morpholin-4-yl-4,5-dihydro-3H-1,3,6-triaza-benzo[e]azulen-6-yl)-(3,4,5,6-tetrahydro-2H-[1,2′]bipyridinyl-4-yl)-methanone;
(3′-Methyl-3,4,5,6-tetrahydro-2H-[1,2′]bipyridinyl-4-yl)-(2-morpholin-4-yl-4,5-dihydro-1H-1,3,6-triaza-benzo[e]azulen-6-yl)-methanone;
(4′-Methyl-3,4,5,6-tetrahydro-2H-[1,2′]bipyridinyl-4-yl)-(2-morpholin-4-yl-4,5-dihydro-3H-1,3,6-triaza-benzo[e]azulen-6-yl)-methanone;
(5′-Methyl-3,4,5,6-tetrahydro-2H-[1,2′]bipyridinyl-4-yl)-(2-morpholin-4-yl-4,5-dihydro-3H-1,3,6-triaza-benzo[e]azulen-6-yl)-methanone;
(9-Fluoro-2-morpholin-4-yl-4,5-dihydro-1H-1,3,6-triaza-benzo[e]azulen-6-yl)-(3,4,5,6-tetrahydro-2H-[1,2′]bipyridinyl-4-yl)-methanone;
(9-Fluoro-2-morpholin-4-yl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(9-Fluoro-2-morpholin-4-yl-4,5-dihydro-1H-1,3,6-triaza-benzo[e]azulen-6-yl)-(4′-methyl-3,4,5,6-tetrahydro-2H-[1,2′]bipyridinyl-4-yl)-methanone;
(9-Fluoro-2-morpholin-4-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(2-Ethyl-9-methyl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(2-Ethyl-9-methyl-4,5-dihydro-3H-1,3,6-triaza-benzo[e]azulen-6-yl)-(4′-methyl-3,4,5,6-tetrahydro-2H-[1,2′]bipyridinyl-4-yl)-methanone;
(2-Ethyl-9-methyl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(9-Methyl-2-propyl-4,5-dihydro-3H-1,3,6-triaza-benzo[e]azulen-6-yl)-(3,4,5,6-tetrahydro-2H-[1,2′]bipyridinyl-4-yl)-methanone;
(9-Methyl-2-propyl-4,5-dihydro-1H-1,3,6-triaza-benzo[e]azulen-6-yl)-(3′-methyl-3,4,5,6-tetrahydro-2H-[1,2′]bipyridinyl-4-yl)-methanone;
(9-Methyl-2-propyl-4,5-dihydro-1H-1,3,6-triaza-benzo[e]azulen-6-yl)-(4′-methyl-3,4,5,6-tetrahydro-2H-[1,2′]bipyridinyl-4-yl)-methanone;
(9-Methyl-2-propyl-4,5-dihydro-3H-1,3,6-triaza-benzo[e]azulen-6-yl)-(5′-methyl-3,4,5,6-tetrahydro-2H-[1,2′]bipyridinyl-4-yl)-methanone;
(2-Cyclopropyl-9-methyl-4,5-dihydro-3H-1,3,6-triaza-benzo[e]azulen-6-yl)-(3,4,5,6-tetrahydro-2H-[1,2′]bipyridinyl-4-yl)-methanone;
(2-Cyclopropyl-9-methyl-4,5-dihydro-3H-1,3,6-triaza-benzo[e]azulen-6-yl)-(5′-methyl-3,4,5,6-tetrahydro-2H-[1,2′]bipyridinyl-4-yl)-methanone;
(2-Butyl-9-methyl-4,5-dihydro-3H-1,3,6-triaza-benzo[e]azulen-6-yl)-(3,4,5,6-tetrahydro-2H-[1,2′]bipyridinyl-4-yl)-methanone;
(2-Butyl-9-methyl-4,5-dihydro-3H-1,3,6-triaza-benzo[e]azulen-6-yl)-(5′-methyl-3,4,5,6-tetrahydro-2H-[1,2′]bipyridinyl-4-yl)-methanone;
(2-Isobutyl-9-methyl-4,5-dihydro-3H-1,3,6-triaza-benzo[e]azulen-6-yl)-(3,4,5,6-tetrahydro-2H-[1,2′]bipyridinyl-4-yl)-methanone;
(2-Isobutyl-9-methyl-4,5-dihydro-3H-1,3,6-triaza-benzo[e]azulen-6-yl)-(5′-methyl-3,4,5,6-tetrahydro-2H-[1,2′]bipyridinyl-4-yl)-methanone;
[9-Methyl-2-(4-methyl-piperazin-1-yl)-4,5-dihydro-1H-1,3,6-triaza-benzo[e]azulen-6-yl]-(5′-methyl-3,4,5,6-tetrahydro-2-[1,2′]bipyridinyl-4-yl)-methanone;
(9-Methyl-2-morpholin-4-yl-4,5-dihydro-1H-1,3,6-triaza-benzo[e]azulen-6-yl)-(3,4,5,6-tetrahydro-2H-[1,2′]bipyridinyl-4-yl)-methanone;
(9-Methyl-2-morpholin-4-yl-4,5-dihydro-3H-1,3,6-triaza-benzo[e]azulen-6-yl)-(4′-methyl-3,4,5,6-tetrahydro-2H-[1,2′]bipyridinyl-4-yl)-methanone;
(4′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)-methanone;
(5′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)-methanone;
(9-Methyl-2-morpholin-4-yl-4,5-dihydro-1H-1,3,6-triaza-benzo[e]azulen-6-yl)-(5′-trifluoromethyl-3,4,5,6-tetrahydro-2H-[1,2′]bipyridinyl-4-yl)-methanone;
(3′-Methyl-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(4′-Methyl-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(5′-Methyl-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(3′-Methoxy-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(4′-Methoxy-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(5′-Methoxy-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(9-Methyl-2-pyrrolidin-1-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(9-Chloro-2-methyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-methyl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(9-Chloro-2-ethyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-propyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-cyclopropyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-cyclohexyl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-piperidin-1-yl-4,5-dihydro-1H-1,3,6-triaza-benzo[e]azulen-6-yl)-(3,4,5,6-tetrahydro-2H-[1,2′]bipyridinyl-4-yl)-methanone;
[9-Chloro-2-(4-methyl-piperazin-1-yl)-4,5-dihydro-1H-1,3,6-triaza-benzo[e]azulen-6-yl]-(3,4,5,6-tetrahydro-2H-[1,2′]bipyridinyl-4-yl)-methanone;
[9-Chloro-2-(4-methyl-piperazin-1-yl)-4,5-dihydro-1H-1,3,6-triaza-benzo[e]azulen-6-yl]-(5′-methyl-3,4,5,6-tetrahydro-2H-[1,2′]bipyridinyl-4-yl)-methanone;
(2-Benzyl-9-chloro-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-morpholin-4-yl-4,5-dihydro-1H-1,3,6-triaza-benzo[e]azulen-6-yl)-(3,4,5,6-tetrahydro-2H-[1,2′]bipyridinyl-4-yl)-methanone;
(9-Chloro-2-morpholin-4-yl-4,5-dihydro-1H-1,3,6-triaza-benzo[e]azulen-6-yl)-(4′-methyl-3,4,5,6-tetrahydro-2H-[1,2′]bipyridinyl-4-yl)-methanone;
(9-Chloro-2-morpholin-4-yl-4,5-dihydro-1H-1,3,6-triaza-benzo[e]azulen-6-yl)-(5′-methyl-3,4,5,6-tetrahydro-2H-[1,2′]bipyridinyl-4-yl)-methanone;
(6′-Methyl-3,4,5,6-tetrahydro-2H-[1,2′]bipyridinyl-4-yl)-(2-morpholin-4-yl-4,5-dihydro-3H-1,3,6-triaza-benzo[e]azulen-6-yl)-methanone;
(2-Ethyl-9-methyl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(9-Methyl-2-propyl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(6′-Methoxy-3,4,5,6-tetrahydro-2H-[1,2′]bipyridinyl-4-yl)-(9-methyl-2-morpholin-4-yl-4,5-dihydro-1H-1,3,6-triaza-benzo[e]azulen-6-yl)-methanone;
(6′-Methyl-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(6′-Methoxy-3,4,5,6-tetrahydro-2H-[1,2′]bipyridinyl-4-yl)-[9-methyl-2-(tetrahydro-pyran-4-yl)-4,5-dihydro-1H-1,3,6-triaza-benzo[e]azulen-6-yl]-methanone;
(9-Chloro-2-morpholin-4-yl-4,5-dihydro-1H-1,3,6-triaza-benzo[e]azulen-6-yl)-(6′-methyl-3,4,5,6-tetrahydro-2H-[1,2′]bipyridinyl-4-yl)-methanone;
(9-Chloro-2-morpholin-4-yl-4,5-dihydro-1H-1,3,6-triaza-benzo[e]azulen-6-yl)-(6′-methoxy-3,4,5,6-tetrahydro-2H-[1,2′]bipyridinyl-4-yl)-methanone;
(9-Methyl-2-morpholin-4-yl-4,5-dihydro-1H-1,3,6-triaza-benzo[e]azulen-6-yl)-(4′-methyl-3,4,5,6-tetrahydro-2H-[1,3′]bipyridinyl-4-yl)-methanone;
(9-Methyl-2-morpholin-4-yl-4,5-dihydro-1H-1,3,6-triaza-benzo[e]azulen-6-yl)-(5′-methyl-3,4,5,6-tetrahydro-2H-[1,3′]bipyridinyl-4-yl)-methanone;
(9-Methyl-2-morpholin-4-yl-4,5-dihydro-3H-1,3,6-triaza-benzo[e]azulen-6-yl)-(2′-methyl-3,4,5,6-tetrahydro-2H-[1,3′]bipyridinyl-4-yl)-methanone;
(9-Chloro-2-methyl-4,5-dihydro-1H-1,3,6-triaza-benzo[e]azulen-6-yl)-(1-phenyl-piperidin-4-yl)-methanone;
(9-Chloro-2-ethyl-4,5-dihydro-1H-1,3,6-triaza-benzo[e]azulen-6-yl)-(1-phenyl-piperidin-4-yl)-methanone;
(9-Chloro-2-propyl-4,5-dihydro-1H-1,3,6-triaza-benzo[e]azulen-6-yl)-(1-phenyl-piperidin-4-yl)-methanone;
(9-Chloro-2-cyclopropyl-4,5-dihydro-1H-1,3,6-triaza-benzo[e]azulen-6-yl)-(1-phenyl-piperidin-4-yl)-methanone;
(9-Chloro-2-cyclohexyl-4,5-dihydro-1H-1,3,6-triaza-benzo[e]azulen-6-yl)-(1-phenyl-piperidin-4-yl)-methanone;
(9-Chloro-2-piperidin-1-yl-4,5-dihydro-1H-1,3,6-triaza-benzo[e]azulen-6-yl)-(1-phenyl-piperidin-4-yl)-methanone;
[9-Chloro-2-(4-methyl-piperazin-1-yl)-4,5-dihydro-1H-1,3,6-triaza-benzo[e]azulen-6-yl]-(1-phenyl-piperidin-4-yl)-methanone;
(9-Chloro-2-methyl-4,5-dihydro-1H-1,3,6-triaza-benzo[e]azulen-6-yl)-[1-(2-fluoro-phenyl)-piperidin-4-yl]-methanone;
(9-Chloro-2-methyl-4,5-dihydro-1H-1,3,6-triaza-benzo[e]azulen-6-yl)-[1-(4-fluoro-phenyl)-piperidin-4-yl]-methanone;
(9-Chloro-2-methyl-4,5-dihydro-1H-1,3,6-triaza-benzo[e]azulen-6-yl)-[1-(2-chloro-phenyl)-piperidin-4-yl]-methanone;
(9-Chloro-2-methyl-4,5-dihydro-1H-1,3,6-triaza-benzo[e]azulen-6-yl)-[1-(4-chloro-phenyl)-piperidin-4-yl]-methanone;
(9-Chloro-2-methyl-4,5-dihydro-1H-1,3,6-triaza-benzo[e]azulen-6-yl)-(1-o-tolyl-piperidin-4-yl)-methanone;
(9-Chloro-2-methyl-4,5-dihydro-1H-1,3,6-triaza-benzo[e]azulen-6-yl)-(1-m-tolyl-piperidin-4-yl)-methanone;
(9-Chloro-2-methyl-4,5-dihydro-1H-1,3,6-triaza-benzo[e]azulen-6-yl)-(1-p-tolyl-piperidin-4-yl)-methanone;
(9-Chloro-2-methyl-4,5-dihydro-1H-1,3,6-triaza-benzo[e]azulen-6-yl)-[1-(3-methoxy-phenyl)-piperidin-4-yl]-methanone;
(9-Chloro-2-methyl-4,5-dihydro-1H-1,3,6-triaza-benzo[e]azulen-6-yl)-[1-(4-methoxy-phenyl)-piperidin-4-yl]-methanone;
(9-Chloro-2-methyl-4,5-dihydro-1H-1,3,6-triaza-benzo[e]azulen-6-yl)-[1-(4-ethyl-phenyl)-piperidin-4-yl]-methanone;
(9-Chloro-2-methyl-4,5-dihydro-1H-1,3,6-triaza-benzo[e]azulen-6-yl)-[1-(4-propyl-phenyl)-piperidin-4-yl]-methanone;
(9-Chloro-2-methyl-4,5-dihydro-1H-1,3,6-triaza-benzo[e]azulen-6-yl)-[1-(4-isopropyl-phenyl)-piperidin-4-yl]-methanone;
(6-Chloro-3-phenyl-4,10-dihydro-3H-2,3,4,9-tetraaza-benzo[f]azulen-9-yl)-(1-pyrimidin-2-yl-piperidin-4-yl)-methanone;
(6-Chloro-3-phenyl-4,10-dihydro-3H-2,3,4,9-tetraaza-benzo[f]azulen-9-yl)-[1-(5-ethyl-pyrimidin-2-yl)-piperidin-4-yl]-methanone;
(3-Methyl-4,10-dihydro-3H-2,3,4,9-tetraaza-benzo[f]azulen-9-yl)-(3,4,5,6-tetrahydro-2H-[1,2]bipyridinyl-4-yl)-methanone;
(3-Ethyl-6-fluoro-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(3-tert-Butyl-6-fluoro-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(3′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(4′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(3′-methoxy-3,4,5,6-tetrahydro-2H-[1,2′]bipyridinyl-4-yl)-methanone;
(3-Benzyl-6-methyl-4,10-dihydro-3H-2,3,4,9-tetraaza-benzo[f]azulen-9-yl)-(4′-methyl-3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(3′-methyl-3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(3′-methoxy-3,4,5,6-tetrahydro-2H-[1,2′]bipyridinyl-4-yl)-methanone;
(6-Chloro-3-ethyl-4,10-dihydro-3H-2,3,4,9-tetraaza-benzo[f]azulen-9-yl)-(3′-methyl-3,4,5,6-tetrahydro-2H-[1,2′]bipyridinyl-4-yl)-methanone;
(6-Chloro-3-ethyl-4,10-dihydro-3H-2,3,4,9-tetraaza-benzo[f]azulen-9-yl)-(4′-methyl-3,4,5,6-tetrahydro-2H-[1,2′]bipyridinyl-4-yl)-methanone;
(6-Chloro-3-ethyl-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(6-Chloro-3-phenyl-4,10-dihydro-3H-2,3,4,9-tetraaza-benzo[f]azulen-9-yl)-(5′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-(6′-methyl-3,4,5,6-tetrahydro-2H-[1,2′]bipyridinyl-4-yl)-methanone;
(6-Chloro-3-methyl-4,10-dihydro-3H-2,3,4,9-tetraaza-benzo[f]azulen-9-yl)-(6′-methyl-3,4,5,6-tetrahydro-2H-[1,2′]bipyridinyl-4-yl)-methanone;
(6-Chloro-3-ethyl-4,10-dihydro-3H-2,3,4,9-tetraaza-benzo[f]azulen-9-yl)-(6′-methyl-3,4,5,6-tetrahydro-2H-[1,2′]bipyridinyl-4-yl)-methanone;
(3,6-Dimethyl-4,10-dihydro-3H-2,3,4,9-tetraaza-benzo[f]azulen-9-yl)-[1-(2-fluoro-phenyl)-piperidin-4-yl]-methanone;
(3,6-Dimethyl-4,10-dihydro-3H-2,3,4,9-tetraaza-benzo[f]azulen-9-yl)-(1-m-tolyl-piperidin-4-yl)-methanone;
(3,6-Dimethyl-4,10-dihydro-3H-2,3,4,9-tetraaza-benzo[f]azulen-9-yl)-(1-p-tolyl-piperidin-4-yl)-methanone;
(3,6-Dimethyl-4,10-dihydro-3H-2,3,4,9-tetraaza-benzo[f]azulen-9-yl)-[1-(3-methoxy-phenyl)-piperidin-4-yl]-methanone;
(3,6-Dimethyl-4,10-dihydro-3H-2,3,4,9-tetraaza-benzo[f]azulen-9-yl)-[1-(4-methoxy-phenyl)-piperidin-4-yl]-methanone;
(3,6-Dimethyl-4,10-dihydro-3H-2,3,4,9-tetraaza-benzo[f]azulen-9-yl)-[1-(4-ethyl-phenyl)-piperidin-4-yl]-methanone;
(3,6-Dimethyl-4,10-dihydro-3H-2,3,4,9-tetraaza-benzo[f]azulen-9-yl)-[1-(4-trifluoromethyl-phenyl)-piperidin-4-yl]-methanone;
(6-Chloro-3-methyl-4,10-dihydro-3H-2,3,4,9-tetraaza-benzo[f]azulen-9-yl)-[1-(2-fluoro-phenyl)-piperidin-4-yl]-methanone;
(6-Chloro-3-methyl-4,10-dihydro-3H-2,3,4,9-tetraaza-benzo[f]azulen-9-yl)-(1-m-tolyl-piperidin-4-yl)-methanone;
(6-Chloro-3-methyl-4,10-dihydro-3H-2,3,4,9-tetraaza-benzo[f]azulen-9-yl)-(1-p-tolyl-piperidin-4-yl)-methanone;
(6-Chloro-3-methyl-4,10-dihydro-3H-2,3,4,9-tetraaza-benzo[f]azulen-9-yl)-[1-(3-methoxy-phenyl)-piperidin-4-yl]-methanone;
(6-Chloro-3-methyl-4,10-dihydro-3H-2,3,4,9-tetraaza-benzo[f]azulen-9-yl)-[1-(4-methoxy-phenyl)-piperidin-4-yl]-methanone;
(6-Chloro-3-methyl-4,10-dihydro-3H-2,3,4,9-tetraaza-benzo[f]azulen-9-yl)-[1-(4-ethyl-phenyl)-piperidin-4-yl]-methanone;
(6-Chloro-3-methyl-4,10-dihydro-3H-2,3,4,9-tetraaza-benzo[f]azulen-9-yl)-[1-(4-trifluoromethyl-phenyl)-piperidin-4-yl]-methanone;
(3,6-Dimethyl-4,10-dihydro-3H-2,3,4,9-tetraaza-benzo[f]azulen-9-yl)-(6′-methyl-3,4,5,6-tetrahydro-2H-[1,3′]bipyridinyl-4-yl)-methanone;
(6-Chloro-3-methyl-4,10-dihydro-3H-2,3,4,9-tetraaza-benzo[f]azulen-9-yl)-(6′-methyl-3,4,5,6-tetrahydro-2H-[1,3′]bipyridinyl-4-yl)-methanone;
(5H,11H-Benzo[e]pyrrolo[1,2-a][1,4]diazepin-10-yl)-(3,4,5,6-tetrahydro-2H-[1,2′]bipyridinyl-4-yl)-methanone;
(5H,11H-Benzo[e]pyrrolo[1,2-a][1,4]diazepin-10-yl)-(5′-methyl-3,4,5,6-tetrahydro-2H-[1,2′]bipyridinyl-4-yl)-methanone;
(5H,11H-Benzo[e]pyrrolo[1,2-a][1,4]diazepin-10-yl)-(3,4,5,6-tetrahydro-2H-[1,3′]bipyridinyl-4-yl)-methanone;
(5H,11H-Benzo[e]pyrrolo[1,2-a][1,4]diazepin-10-yl)-(1-pyrazin-2-yl-piperidin-4-yl)-methanone;
(5H,11H-Benzo[e]pyrrolo[1,2-a][1,4]diazepin-10-yl)-(1-phenyl-piperidin-4-yl)-methanone;
(7-Fluoro-5H,11H-benzo[e]pyrrolo[1,2-a][1,4]diazepin-10-yl)-(3,4,5,6-tetrahydro-2H-[1,2′]bipyridinyl-4-yl)-methanone;
(7-Fluoro-5H,11H-benzo[e]pyrrolo[1,2-a][1,4]diazepin-10-yl)-(1-phenyl-piperidin-4-yl)-methanone;
(7-Methyl-5H,11H-benzo[e]pyrrolo[1,2-a][1,4]diazepin-10-yl)-(3,4,5,6-tetrahydro-2H-[1,2′]bipyridinyl-4-yl)-methanone;
(7-Methyl-5H,11H-benzo[e]pyrrolo[1,2-a][1,4]diazepin-10-yl)-(1-phenyl-piperidin-4-yl)-methanone;
(5H,11H-Benzo[e]pyrrolo[1,2-a][1,4]diazepin-10-yl)-[1-(2-fluoro-phenyl)-piperidin-4-yl]-methanone;
(5H,11H-Benzo[e]pyrrolo[1,2-a][1,4]diazepin-10-yl)-[1-(3-fluoro-phenyl)-piperidin-4-yl]-methanone;
(5H,11H-Benzo[e]pyrrolo[1,2-a][1,4]diazepin-10-yl)-[1-(4-fluoro-phenyl)-piperidin-4-yl]-methanone;
(5H,11H-Benzo[e]pyrrolo[1,2-a][1,4]diazepin-10-yl)-[1-(2-chloro-phenyl)-piperidin-4-yl]-methanone;
(5H,11H-Benzo[e]pyrrolo[1,2-a][1,4]diazepin-10-yl)-[1-(4-chloro-phenyl)-piperidin-4-yl]-methanone;
(5H,11H-Benzo[e]pyrrolo[1,2-a][1,4]diazepin-10-yl)-(1-o-tolyl-piperidin-4-yl)-methanone;
(5H,11H-Benzo[e]pyrrolo[1,2-a][1,4]diazepin-10-yl)-(1-m-tolyl-piperidin-4-yl)-methanone;
(5H,11H-Benzo[e]pyrrolo[1,2-a][1,4]diazepin-10-yl)-(1-p-tolyl-piperidin-4-yl)-methanone;
(5H,11H-Benzo[e]pyrrolo[1,2-a][1,4]diazepin-10-yl)-[1-(2-methoxy-phenyl)-piperidin-4-yl]-methanone;
(5H,11H-Benzo[e]pyrrolo[1,2-a][1,4]diazepin-10-yl)-[1-(3-methoxy-phenyl)-piperidin-4-yl]-methanone;
(5H,11H-Benzo[e]pyrrolo[1,2-a][1,4]diazepin-10-yl)-[1-(4-methoxy-phenyl)-piperidin-4-yl]-methanone;
(5H,11H-Benzo[e]pyrrolo[1,2-a][1,4]diazepin-10-yl)-[1-(4-ethyl-phenyl)-piperidin-4-yl]-methanone;
(5H,11H-Benzo[e]pyrrolo[1,2-a][1,4]diazepin-10-yl)-[1-(4-propyl-phenyl)-piperidin-4-yl]-methanone;
(5H,11H-Benzo[e]pyrrolo[1,2-a][1,4]diazepin-10-yl)-[1-(4-isopropyl-phenyl)-piperidin-4-yl]-methanone;
(5H,11H-Benzo[e]pyrrolo[1,2-a][1,4]diazepin-10-yl)-[1-(4-trifluoromethyl-phenyl)-piperidin-4-yl]-methanone;
3-[4-(5H,11H-Benzo[e]pyrrolo[1,2-a][1,4]diazepine-10-carbonyl)-piperidin-1-yl]-benzonitrile;
(5H,11H-Benzo[e]pyrrolo[1,2-a][1,4]diazepin-10-yl)-[1-(3-nitro-phenyl)-piperidin-4-yl]-methanone;
[1-(2-Amino-phenyl)-piperidin-4-yl]-(5H,11H-benzo[e]pyrrolo[1,2-a][1,4]diazepin-10-yl)-methanone;
[1-(4-Amino-phenyl)-piperidin-4-yl]-(5H,11H-benzo[e]pyrrolo[1,2-a][1,4]diazepin-10-yl)-methanone;
[1-(2-Aminomethyl-phenyl)-piperidin-4-yl]-(5H,11H-benzo[e]pyrrolo[1,2-a][1,4]diazepin-10-yl)-methanone;
and a pharmaceutically acceptable salt or solvate thereof.

14. A compound according to claim 1, or a pharmaceutically acceptable salt or solvate thereof, for use in therapy.

15. The use of a compound according to claim 1, or a pharmaceutically acceptable salt or solvate thereof, in the manufacture of a medicament for the treatment of a disease or condition mediated by vasopressin V₁ₐ receptors.

16. A method of treatment of a disease or condition mediated by vasopressin V₁ₐ receptors comprising administration to a subject in need thereof a therapeutically effective amount of a compound according to claim 1, or a pharmaceutically acceptable salt or solvate thereof.

17. The use of claim 15 or the method of claim 16 wherein the disease or condition mediated by vasopressin V₁ₐ receptors is selected from dysmenorrhoea (primary dysmenorrhoea and/or secondary dysmenorrhoea), pre-term labour, hypertension, Raynaud's disease, brain oedema, motion sickness, hyperlipemia, small cell lung cancer, depression, anxiety, hyponatremia, liver cirrhosis and congestive heart failure.

18. A pharmaceutical composition comprising a compound according to claim 1, or a pharmaceutically acceptable salt or solvate thereof, and a pharmaceutically acceptable carrier, diluent or excipient.
